(19) United States
(12) Patent Application Publication
(10) Pub. No.: US 2025/0171473 A1
(43) Pub. Date: May 29, 2025

(54) Phosphorous Containing BCL Inhibitors and Senolytic Compounds and Uses Thereof

(71) Applicant: Rubedo Life Sciences, Inc., Sunnyvale, CA (US)

(72) Inventors: Michael Hadd, San Jose, CA (US); Andrew John Jennings, San Jose, CA (US)

(21) Appl. No.: 18/960,760

(22) Filed: Nov. 26, 2024

Related U.S. Application Data
(60) Provisional application No. 63/602,720, filed on Nov. 27, 2023.

(51) Int. Cl. C07F 9/6558; A61K 31/675; A61K 31/706; C07H 15/26

(52) U.S. Cl. CPC C07F 9/65583; A61K 31/675; A61K 31/706; C07H 15/26

(57) ABSTRACT

The present invention relates to compounds that kill senescent cells i.e., senolytic compounds and compounds which inhibit BCL-2 protein family (BCL-2, BCL-XL, BCL-W, MCL-1, A1, BCL-B). The present invention also provides compounds and methods for treating senescence-associated diseases or disorders, and compounds and methods for treating diseases or disorders impacted by BCL2 protein family.

### CROSS REFERENCE TO RELATED APPLICATIONS

This application claims priority to U.S. Provisional Application Ser. No. 63/602,720, filed Nov. 27, 2023, under 35 U.S.C. § 119 (e) which is herein incorporated by reference in its entirety for all purposes.

### TECHNICAL FIELD

The present invention relates to compounds that kill senescent cells i.e., senolytic compounds and compounds which inhibit BCL-2 protein family (BCL-2, BCL-XL, BCL-W, MCL-1, A1, BCL-B). The present invention also provides compounds and methods for treating senescence-associated diseases or disorders, and compounds and methods for treating diseases or disorders impacted by BCL2 protein family.

### BACKGROUND

Senescence is a cellular program that imposes a stable arrest on damaged or old cells to prevent replication of these cells. As well as growth arrest, senescent cells undergo profound phenotypic changes that include chromatin reorganization, increase of beta-galactosidase activity (referred to as senescence-associated β-galactosidase or SA-β-Gal) and secretion of multiple factors, mainly pro-inflammatory, that are collectively referred to as the senescence-associated secretory phenotype (SASP).

Replicative senescence is activated upon serial passage of cells in culture (or as cells become older in an organism). Senescence is also induced by, for example, oncogene activation, irradiation and exposure to chemotherapeutic drugs. In addition, there are several drugs, the prototypic example being CDK4/CDK6 inhibitors such as Palbociclib, which induce senescence.

The stable growth arrest characteristic of senescence is implemented by the activation of the p16/R^{b} and p53/p21 pathways. The cyclin-dependent kinase inhibitors p^{16INK4a} and p21Cip1 inhibit CDK activity, resulting in R^{b} hypophosphorylation and G1 growth arrest (Kuilman et al., Genes Dev 2010 24, 2453-2479). Moreover, p^{16INK4a} is specifically induced during senescence and used to identify senescent cells alone or in combination with other markers such as SA-β-Gal activity, formation of senescence-associated heterochromatin foci (SAHF) and others.

Senescent cells accumulate during age and are associated with many diseases, including cancer, fibrosis and many age-related pathologies. Recent evidence suggests that senescent cells are detrimental in multiple pathologies and their elimination confers many advantages, ameliorating multiple pathologies and increasing lifespan.

Senescent cells are present in many pre-neoplastic lesions, fibrotic tissues (e.g., in the liver, kidney, heart, pancreas) and old tissues. Senescent cells are also associated with a long list of other pathologies including neurological (e.g., brain aneurysm, Alzheimer's and Parkinson), pulmonary (e.g., idiopathic pulmonary fibrosis, chronic obstructive pulmonary disease and cystic fibrosis), ophthalmological (e.g., cataracts, glaucoma, macular degeneration), musculoskeletal (e.g., sarcopenia, disc degeneration, osteoarthritis), cardiovascular (e.g. atherosclerosis, cardiac fibrosis, aorta aneurysm), renal (e.g., kidney disease, transplant complications) and others such as diabetes, mucositis, hypertension and osteomyelofibrosis (OMF). While senescent cells have a protective role against cancer and limit most types of fibrosis, the accumulation of senescent cells during ageing and many other diseases is believed to be detrimental.

Evidence for the many detrimental effects of senescent cells (and the benefits caused by their selective elimination) was provided by a series of studies from the van Deursen lab (Baker et al., Nature 2016 530, 184-189; Baker et al., Nature 2011 479, 232-236; Childs et al., Science 2016 354, 472-477). These studies used transgenic mice (INK4-ATTAC mice) that express an inducible fusion protein specifically on senescent cells (taking advantage of the promoter of p16Ink4a). Activation of this fusion protein, by addition of a drug that triggers its dimerization, results in the selective death of senescent cells.

The clearance of senescent cells increases lifespan by attenuating multiple age-related pathologies (Baker et al., Nature 2016 530, 184-189; Baker et al., Nature 2011 479, 232-236; Childs et al., 2016 Science 354, 472-477) by use of the above mice model. Clearance of senescent cells delayed tumorigenesis, and attenuated cataract formation, atherosclerosis and the age-related deterioration of kidney, fat and heart amongst other organs. The results obtained with INK4-ATTAC mice have been replicated in part in a different mouse model in which a tk transgene is expressed in senescent cells (3MR mice), allowing for their selective elimination upon ganciclovir treatment (Demaria et al., Dev Cell 2014 31, 722-733). Moreover, the elimination of senescent cells in chemotherapy reduced cancer recurrence and the side effects associated with chemotherapy. Importantly, the elimination of senescent cells has no side effects besides delaying wound healing if the senescent cells are eliminated during the healing process (Baker et al., Nature 2016 530, 184-189; Demaria et al., Dev Cell 2014 31, 722-733). However, the unifying prevalent hypothesis is that (pro-inflammatory) factors secreted by senescent cells hamper tissue homeostasis. This suggests that common mechanisms, mediated by senescent cells, could be responsible for the effects of many age-related pathologies.

Proof-of-concept studies have led to the identification of compounds that can selectively eliminate senescent cells (so-called “senolytics”). Several senolytic compounds have been identified to date, including dasatinib and quercetin, piperlongumine and BCL2 protein family inhibitors such as ABT-263 and ABT-737. Currently BCL2 protein family inhibitors are the most promising senolytics, having been shown to kill a range of senescent cells in vivo, with reproducible effects in transgenic mice. BCL2 family inhibitors were initially developed as therapies for lymphoma. ABT-737 is a small molecule inhibitor of BCL-2, BCL-XL and BCL-w but has low solubility and oral bioavailability. ABT-263 inhibits the same molecules and is better suited for use in vivo but causes significant thrombocytopenia as a side-effect.

Accordingly, there is a need to identify more compounds, and classes of compounds with senolytic properties and/or compounds which inhibit BCL2 protein family.

### SUMMARY

In one aspect, a compound of the structure below which satisfies these and other needs is provided: In one aspect, a compound of structure:

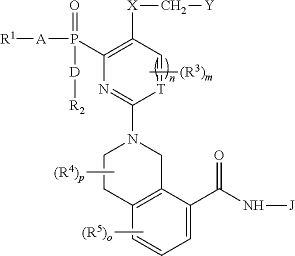

or salts, hydrates and solvates thereof is provided wherein, X is heteroaryl or substituted heteroaryl; Y is alkyl, substituted alkyl, cycloalkyl, substituted cycloalkyl, heteroalkyldiyl or substituted heteroalkyldiyl; A is —O— or —NR⁶—; D is —O— or absent; J is heteroaryl or substituted heteroaryl; R¹, R² and R⁶ are independently-H, alkyl, alkenyl, aryl, substituted aryl, arylalkyl, substituted arylalkyl, cycloalkyl, substituted cycloalkyl, cycloalkenyl or substituted cycloalkenyl wherein substituted arylalkyl includes substitution with a carbohydrate derivative; ach of R³ and Re⁵ are independently halo, alkyl, alkenyl, alkynyl or haloalkyl; R⁴ is halo, alkyl, alkenyl, alkynyl or haloalkyl or alternatively two R⁴ attached to the carbon atom form a cycloalkyl or cycloalkenyl ring; m is 0, 1, 2 or 3; n is 0 or 1; o is 0, 1, 2 or 3; and p is 0, 1, 2 or 3.

In another aspect, a compound of the structure below which satisfies these and other needs is provided:

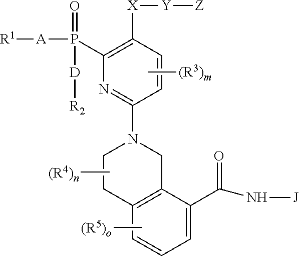

or salts, hydrates and solvates thereof where X is heteroaryl or substituted heteroaryl; Y is alkyldiyl, substituted alkyldiyl, heteroalkyldiyl or substituted heteroalkyldiyl; Z is cycloalkyl, substituted cycloalkyl, cycloalkenyl, substituted cycloalkenyl, aryl, substituted aryl, cycloheteroalkyl or substituted cycloheteroalkyl; A is —O— or —NR⁶—; D is —O— or absent; J is heteroaryl or substituted heteroaryl; R¹, R² and R⁶ are independently-H alkyl, alkenyl, aryl, substituted aryl, arylalkyl, substituted arylalkyl cycloalkyl, substituted cycloalkyl, cycloalkenyl or substituted cycloalkenyl wherein substituted arylalkyl includes substitution with a carbohydrate derivative; each R³ and R⁵ are independently halo, alkyl, alkenyl, alkynyl or haloalkyl; R⁴ is halo, alkyl, alkenyl, alkynyl or haloalkyl or alternatively two R⁴ attached to the carbon atom form a cycloalkyl or cycloalkenyl ring; m is 0, 1, 2 or 3, n is 0, 1, 2, 3, 4, 5 or 6; and o is 0, 1, 2 or 3.

In yet another aspect a compound of the structure below is provided:

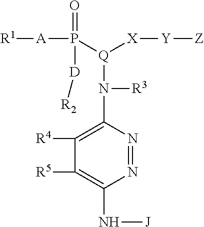

or salts, hydrates and solvates thereof where Q is heteroaryl or substituted heteroaryl; X is alkyldiyl, substituted alkyldiyl, heteroalkyldiyl or substituted heteroalkyldiyl; Y is —O—, —NR⁶—, or —CH₂—; Z is cycloalkyl, substituted cycloalkyl, cycloalkenyl, substituted cycloalkenyl, aryl, substituted aryl, cycloheteroalkyl, substituted cycloheteroalkyl heteroaryl or substituted heteroaryl; A is —O— or —NR⁷—; D is —O— or absent; J is heteroaryl or substituted heteroaryl; R¹, R², R³ and R⁷ are independently-H, alkyl, alkenyl, aryl, substituted aryl, arylalkyl or substituted arylalkyl where substituted arylalkyl includes substitution with a carbohydrate derivative; R⁴ and R⁵ are independently-H, alkyl, substituted alkyl, alkenyl, substituted alkenyl, cycloalkyl, substituted cycloalkyl or together with the atoms to which they are bonded form a cycloalkenyl or substituted cycloalkenyl group; and R⁶ is —H or alkyl.

Also provided are derivatives, including salts, esters, enol ethers, enol esters, solvates, hydrates, metabolites and prodrugs of the compounds described herein. Further provided are pharmaceutical compositions which include the compounds provided herein and a pharmaceutically acceptable vehicle.

In still another aspect, methods of treating, preventing, or ameliorating symptoms of medical disorders such as, for example, senescence-associated diseases or disorders, and diseases or disorders impacted by BCL are provided.

### DETAILED DESCRIPTION

### Definitions

Unless defined otherwise, all technical and scientific terms used herein have the same meaning as is commonly understood by one of ordinary skill in the art to which this invention belongs. If a plurality of definitions for a term exist herein, those in this section prevail unless stated otherwise.

As used in the specification and the appended claims, the indefinite articles “a” and “an” and the definite article “the” can include plural referents as well as singular referents unless specifically stated otherwise or the context clearly indicates otherwise. The term “exemplary” as used herein means “serving as an example, instance or illustration”. Any embodiment or feature characterized herein as “exemplary” is not necessarily to be construed as preferred of advantageous over other embodiments or features.

Where elements are presented in list format (e.g., in a Markush group), it is understood that each possible subgroup of the elements is also disclosed, and any one or more elements can be removed from the list or group.

It is further understood that the disclosure of a numerical range is a specific disclosure of all the possible subranges and all the possible individual numbers (whether whole numbers or fractions) within that range regardless of the breadth of that range. It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment or compound found within the prior art, can be explicitly excluded from the claims, regardless of whether or not the specific exclusion is recited in the specification.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

As used herein, and unless otherwise specified, the terms “about” and “approximately,” when used in connection with a property with a numeric value or range of values indicate that the value or range of values may deviate to an extent deemed reasonable to one of ordinary skill in the art while still describing the particular property. Specifically, the terms “about” and “approximately,” when used in this context, indicate that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cu-v” indicates that the following group has from u to v carbon atoms. It should be understood that u to v carbons includes u+1 to v, u+2 to v, u+3 to v, etc. carbons, u+1 to u+3 to v, u+1 to u+4 to v, u+2 to u+4 to v, etc. and cover all possible permutation of u and v.

“Alkyl,” by itself or as part of another substituent, refers to a saturated, branched, or straight-chain monovalent hydrocarbon radical derived by the removal of one hydrogen atom from a single carbon atom of a parent alkane. Typical alkyl groups include, but are not limited to, methyl; ethyl; propyls such as propan-1-yl, propan-2-yl, etc.; butyls such as butan-1-yl, butan-2-yl, 2-methyl-propan-1-yl, 2-methyl-propan-2-yl, etc.; and the like. In some aspects, an alkyl group comprises from 1 to 20 carbon atoms (C1-C20 alkyl). In other aspects, an alkyl group comprises from 1 to 10 carbon atoms (C1-C10 alkyl). In still other aspects, an alkyl group comprises from 1 to 6 carbon atoms (C₁-C₆ alkyl).

“Alkenyl,” by itself or as part of another substituent, refers to an unsaturated branched, straight-chain having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, etc.; and the like. In some aspects, an alkenyl group comprises from 2 to 20 carbon atoms (C₂-C₂₀ alkenyl). In other aspects, an alkenyl group comprises from 2 to 10 carbon atoms (C₂-C₁₀ alkenyl). In still other aspects, an alkenyl group comprises from 2 to 6 carbon atoms (C₂-C₆ alkenyl).

“Alkynyl,” by itself or as part of another substituent refers to an unsaturated branched, straight-chain having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In some aspects, an alkynyl group comprises from 2 to 20 carbon atoms (C₂-C₂₀ alkynyl). In other aspects, an alkynyl group comprises from 2 to 10 carbon atoms (C₂-C₁₀ alkynyl). In still other aspects, an alkynyl group comprises from 2 to 6 carbon atoms (C₂-C₆ alkynyl).

Alkyldiyl” by itself or as part of another substituent, refers to a saturated, branched or straight-chain hydrocarbon group derived by the removal of one hydrogen atom from each of two different carbon atoms of a parent alkane, or by the removal of two hydrogen atoms from a single carbon atom of a parent alkane. The two monovalent radical centers or each valency of the divalent radical center can form bonds with the same or different atoms. Typical alkyldiyl groups include, but are not limited to methandiyl; ethyldiyls such as ethan-1,1-diyl, ethan-1,2-diyl; propyldiyls such as propan-1,1-diyl, propan-1,2-diyl, propan-2,2-diyl, propan-1,3-diyl, butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, etc.; and the like. In some embodiments, the alkyldiyl group is (C₁-C₂₀) alkyldiyl. In other embodiments, the alkyldiyl group is (C₁-C₁₀) alkyldiyl. In still other embodiments, the alkyldiyl group is (C₁-C₆) alkyldiyl.

“Alkenyldiyl” by itself or as part of another substituent, refers to a unsaturated, branched, straight-chain or cyclic divalent hydrocarbon group derived by the removal of one hydrogen atom from each of two different carbon atoms of a parent alkene, or by the removal of two hydrogen atoms from a single carbon atom of a parent alkene. The two monovalent radical centers or each valency of the divalent radical center can form bonds with the same or different atoms. Typical alkenyldiyl groups include, but are not limited to ethen-1,1-diyl, prop-1-en-1,1-diyl, prop-1-en-1,2-diyl, butyldiyls such as, but-1-en-1,1-diyl, but-1-en-1,2-diyl, but-1-en-1,3-diyl, but-1-en-1,4-diyl, 2-methyl-prop-1-en-1,1-diyl, etc.; and the like. In some embodiments, the alkenyldiyl group is (C₂-C₂₀) alkenyldiyl. In other embodiments, the alkenyldiyl group is (C₂-C₁₀) alkenyldiyl. In still other embodiments, the alkenyldiyl group is (C₂-C₆) alkenyldiyl.

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like. In some aspects, an aryl group comprises from 6 to 30 carbon atoms (C₆-C₃₀ aryl). In other aspects, an aryl group comprises from 6 to 20 carbon atoms (C₆-C₂₀ aryl). In still other aspects, an aryl group comprises from 6 to 15 carbon atoms (C₆-C₁₅ aryl). In still other aspects, an aryl group comprises from 6 to 10 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 1-phenylethen-1-yl, naphthylmethyl, 2-naphthylethan-1-yl, 1-naphthylethen-1-yl, naphthobenzyl, 2-naphthophenylethan-1-yl and the like. In some aspects, an arylalkyl group is (C₇-C₄₀) arylalkyl, e.g., the alkyl moiety of the arylalkyl group is (C₁-C₁₀) alkyl and the aryl moiety is (C₆-C₃₀) aryl. In other aspects, an arylalkyl group is (C₇-C₃₀) arylalkyl, e.g., the alkyl moiety of the arylalkyl group is (C₁-C₁₀) alkyl and the aryl moiety is (C₆-C₂₀) aryl. In other aspects, an arylalkyl group is (C₇-C₂₀) arylalkyl, e.g., the alkyl moiety of the arylalkyl group is (C₁-C₈) alkyl and the aryl moiety is (C₆-C₁₂) aryl. In still other aspects, an arylalkyl group is (C₇-C₁₅) arylalkyl, e.g., the alkyl moiety of the arylalkyl group is (C₁-C₅) alkyl and the aryl moiety is (C₆-C₁₀) aryl.

“Arylalkenyl,” by itself or as part of another substituent, refers to an acyclic alkenyl group in which one of the hydrogen atoms bonded to a carbon atom, is replaced with an aryl group as, as defined herein. In some aspects, an arylalkenyl group is (C₈-C₄₀) arylalkenyl, e.g., the alkenyl moiety of the arylalkenyl group is (C₂-C₁₀) alkenyl and the aryl moiety is (C₆-C₃₀) aryl. In other aspects, an arylalkenyl group is (C₈-C₃₀) arylalkenyl, e.g., the alkenyl moiety of the arylalkenyl group is (C₂-C₁₀) alkenyl and the aryl moiety is (C₈-C₂₀) aryl. In other aspects, an arylalkenyl group is (C₈-C₂₀) arylalkenyl, e.g., the alkenyl moiety of the arylalkenyl group is (C₂-C₈) alkenyl and the aryl moiety is (C₆-C₁₂) aryl. In still other aspects, an arylalkenyl group is (C₈-C₁₅) arylalkenyl, e.g., the alkenyl moiety of the arylalkenyl group is (C₂-C₅) alkenyl and the aryl moiety is (C₆-C₁₀) aryl.

“Arylalkynyl,” by itself or as part of another substituent, refers to an acyclic alkynyl group in which one of the hydrogen atoms bonded to a carbon atom, is replaced with an aryl group as, as defined herein. In some aspects, an arylalkynyl group is (C₈-C₄₀) arylalkynyl, e.g., the alkynyl moiety of the arylalkynyl group is (C₂-C₁₀) alkynyl and the aryl moiety is (C₆-C₃₀) aryl. In other aspects, an arylalkynyl group is (C₈-C₃₀) arylalkynyl, e.g., the alkynyl moiety of the arylalkynyl group is (C₂-C₁₀) alkynyl and the aryl moiety is (C₆-C₂₀) aryl. In other aspects, an arylalkynyl group is (C₈-C₂₀) arylalkynyl, e.g., the alkynyl moiety of the arylalkenyl group is (C₂-C₈) alkynyl and the aryl moiety is (C₆-C₁₂) aryl. In still other aspects, an arylalkynyl group is (C₈-C₁₅) arylalkynyl, e.g., the alkynyl moiety of the arylalkynyl group is (C₂-C₅) alkynyl and the aryl moiety is (C₆-C₁₀) aryl.

“Carbohydrate derivative,” refers to carbohydrates, of general formula CₙH₂ₙOₙ attached to a group of a chemical compound. In some embodiments a carbohydrate derivative typically contain five or six carbon atoms. In other embodiments, a carbohydrate derivative is a monosaccharide (e.g., glucose, fructose, galactose, ribose,). In still other embodiments, a carbohydrate derivative includes disaccharides (e.g., lactose, sucrose, maltose, cellobiose, chitobiose, gentobiose, etc.). In still other embodiments, a carbohydrate derivative includes oligosaccharides (e.g., oligofructose, oligogalactose, raffinose, plantose, veracose, etc.). In still other embodiments, a carbohydrate derivative includes polysaccharides (e.g., cellulose, amylose, starch, chitin, pectins, galactogen, etc.). In still other embodiments, a carbohydrate derivative includes protected carbohydrates, such as for example, esters (e.g., acetates or benzoates, etc.), silyl derivatives or carbohydrates protected with any other known alcohol protecting groups.

“Compounds,” refers to compounds encompassed by structural formulae disclosed herein and includes any specific compounds within these formulae whose structure is disclosed herein. Compounds may be identified either by their chemical structure and/or chemical name. The chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass the stereoisomerically pure form depicted in the structure (e.g., geometrically pure, enantiomerically pure or diastereomerically pure). The chemical structures depicted herein also encompass the enantiomeric and stereoisomeric derivatives of the compound depicted. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds may also be atropisomers. The compounds described also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

“Cycloalkyl,” by itself or as part of another substituent, refers to a saturated cyclic monovalent hydrocarbon radical derived by the removal of one hydrogen atom from a single carbon atom of a parent cycloalkane. Typical cycloalkyl groups include, but are not limited to, cyclopropyl, cyclobutyl, cyclopentyl cyclopentenyl; etc.; and the like. In some aspects, a cycloalkyl group comprises from 3 to 20 carbon atoms (C₃-C₁₅ cycloalkyl). In other aspects, a cycloalkyl group comprises from 3 to 10 carbon atoms (C₃-C₁₀ cycloalkyl). In still other aspects, a cycloalkyl group comprises from 3 to 8 carbon atoms (C₃-C₈ cycloalkyl). The term “cyclic monovalent hydrocarbon radical” also includes multicyclic hydrocarbon ring systems having a single radical and between 5 and 12 carbon atoms. Exemplary multicyclic cycloalkyl rings include, for example, norbornyl, pinyl, and adamantyl.

“Cycloalkyldiyl” by itself or as part of another substituent, refers to a cyclic hydrocarbon group derived by the removal of one hydrogen atom from each of two different carbon atoms of a parent cycloalkane, or by the removal of two hydrogen atoms from a single carbon atom of a parent cycloalkane. The two monovalent radical centers or each valency of the divalent radical center can form bonds with the same or different atoms. Exemplary multicyclic cycloalkyldiyl rings include, for example, norbornyldiyl, pinyldiyl, and adamantyldiyl

“Cycloalkenyl,” by itself or as part of another substituent, refers to an unsaturated cyclic monovalent hydrocarbon radical derived by the removal of one hydrogen atom from a single carbon atom of a parent cycloalkene. Typical cycloalkenyl groups include, but are not limited to, cyclopropenyl, cyclobutenyl, cyclopentenyl; etc.; and the like. In some aspects, a cycloalkenyl group comprises from 3 to 20 carbon atoms (C₃-C₂₀ cycloalkenyl). In other aspects, a cycloalkenyl group comprises from 3 to 10 carbon atoms (C₃-C₁₀ cycloalkenyl). In still other aspects, a cycloalkenyl group comprises from 3 to 8 carbon atoms (C₃-C₈ cycloalkenyl).

“Cycloheteroalkyl,” by itself or as part of another substituent, refers to a cycloalkyl group as defined herein in which one or more of the carbon atoms (and optionally any associated hydrogen atoms), are each, independently of one another, replaced with the same or different heteroatoms or heteroatomic groups as defined in “heteroalkyl” below. In some aspects, a cycloheteroalkyl group comprises from 3 to 20 carbon and hetero atoms (3-20 cycloheteroalkyl). In other aspects, a cycloheteroalkyl group comprises from 3 to 10 carbon and hetero atoms (3-10 cycloheteroalkyl). In still other aspects, a cycloheteroalkyl group comprises from 3 to 8 carbon and hetero atoms (3-8 cycloheteroalkyl). The term “cyclic monovalent heteroalkyl radical” also includes multicyclic heteroalkyl ring systems having a single radical and between 3 and 12 carbon and at least one hetero atom. Exemplary cycloheteroalkyl groups include, for example, azetidine, pyrrolidine, piperazine, piperidine, morpholine and tetrahydrofuran.

“Cycloheteroalkenyl,” by itself or as part of another substituent, refers to a cycloalkenyl group as defined herein in which one or more of the carbon atoms (and optionally any associated hydrogen atoms), are each, independently of one another, replaced with the same or different heteroatoms or heteroatomic groups as defined in “heteroalkenyl” below. In some aspects, a cycloheteroalkenyl group comprises from 3 to 20 carbon and hetero atoms (3-20 cycloheteroalkenyl). In other aspects, a cycloheteroalkenyl group comprises from 3 to 10 carbon and hetero atoms (3-10) cycloheteroalkenyl). In still other aspects, a cycloheteroalkenyl group comprises from 3 to 8 carbon and heteroatoms (3-8 cycloheteroalkenyl). The term “cyclic monovalent heteroalkenyl radical” also includes multicyclic heteroalkenyl ring systems having a single radical and between 2 and 12 carbon and at least one hetero atom.

“Halo,” by itself or as part of another substituent refers to a radical-F, —C₁, —Br or—I.

“Heteroalkyl,” refer to an alkyl group, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰², ═N—N═, —N—N—, —N═N—NR⁵⁰³R⁴⁰⁴, —PR⁵⁰⁵—, —P(O)₂—, —POR⁵⁰⁶—, —O—P(O)₂—, —SO—, —SO₂—, —SnR⁵⁰⁷R⁵⁰⁸ and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, aryl, substituted aryl, heteroalkyl, heteroaryl or substituted heteroaryl. In some aspects, an heteroalkyl group comprises from 1 to 20 carbon and hetero atoms (₁₋₂₀ heteroalkyl). In other aspects, an heteroalkyl group comprises from 1 to 10 carbon and hetero atoms (₁₋₁₀ heteroalkyl). In still other aspects, an heteroalkyl group comprises from 1 to 6 carbon and hetero atoms (₁₋₆ heteroalkyl).

“Heteroalkenyl,” refers to an alkenyl group in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰², ═N—N═, —N═N—, —N═N—NR⁵⁰³R⁴⁰⁴, —PR⁵⁰⁵—, —P(O)₂—, —POR⁵⁰⁶—, —O—P(O)₂—, —SO—, —SO₂—, —SnR⁵⁰⁷R^{50 8} and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, aryl, substituted aryl, heteroalkyl, heteroaryl or substituted heteroaryl. In some aspects, an heteroalkenyl group comprises from 1 to 20 carbon and hetero atoms (₁₋₂₀ heteroalkenyl). In other aspects, an heteroalkenyl group comprises from 1 to 10 carbon and hetero atoms (₁₋₁₀ heteroalkenyl). In still other aspects, an heteroalkenyl group comprises from 1 to 6 carbon and hetero atoms (₁₋₆ heteroalkenyl).

“Heteroalkyldiyl” by itself or as part of another substituent, refers to a saturated, branched or straight-chain hydrocarbon group derived by the removal of one hydrogen atom from each of two different carbon atoms of a parent heteroalkane, or by the removal of two hydrogen atoms from a single carbon atom of a parent heteroalkane. The two monovalent radical centers or each valency of the divalent radical center can form bonds with the same or different atoms. In some embodiments, the heteroalkyldiyl group is (C₁-C₂₀) heteroalkyldiyl. In other embodiments, the heteroalkyldiyl group is (C₁-C₁₀) heteroalkyldiyl. In still other embodiments, thehetero alkyldiyl group is (C₁-C₆) heteroalkyldiyl.

“Heteroalkenyldiyl” by itself or as part of another substituent, refers to a unsaturated, branched, straight-chain or cyclic divalent heteroalkenyl group derived by the removal of one hydrogen atom from each of two different carbon atoms of a parent heteroalkene, or by the removal of two hydrogen atoms from a single carbon atom of a parent heteroalkene. The two monovalent radical centers or each valency of the divalent radical center can form bonds with the same or different atoms. In some embodiments, the heteroalkenyldiyl group is (C₂-C₂₀) heteroalkenyldiyl. In other embodiments, the heteroalkenyldiyl group is (C₂-C₁₀) heteroalkenyldiyl. In still other embodiments, the heteroalkenyldiyl group is (C₂-C₆) heteroalkenyldiyl.

“Heteroaryl,” by itself or as part of another substituent, refers to a monovalent heteroaromatic radical derived by the removal of one hydrogen atom from a single atom of a parent heteroaromatic ring system, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some aspects, the heteroaryl group comprises from 5 to 20 ring atoms (5-20 membered heteroaryl). In other aspects, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl,” by itself or as part of another substituent refers to an acyclic alkyl group in which one of the hydrogen atoms bonded to a carbon atom, typically a terminal or sp³ carbon atom, is replaced with a heteroaryl group. In some aspects, the heteroarylalkyl group is a 6-21 membered heteroarylalkyl, e.g., the alkyl moiety of the heteroarylalkyl is (C₁-C₆) alkyl and the heteroaryl moiety is a 5-15-membered heteroaryl. In other aspects, the heteroarylalkyl is a 6-13 membered heteroarylalkyl, e.g., the alkyl moiety is (C₁-C₃) alkyl and the heteroaryl moiety is a 5-10 membered heteroaryl.

“Heteroarylalkenyl,” by itself or as part of another substituent refers to an acyclic alkenyl group in which one of the hydrogen atoms bonded to a carbon atom, is replaced with a heteroaryl group. In some aspects, the heteroarylalkenyl group is a 7-21 membered heteroarylalkenyl, e.g., the alkenyl moiety of the heteroarylalkenyl is (C₂-C₆) alkenyl and the heteroaryl moiety is a 5-15-membered heteroaryl. In other aspects, the heteroarylalkenyl is a 7-13 membered heteroarylalkenyl, e.g., the alkenyl moiety is (C₂-C₃) alkenyl and the heteroaryl moiety is a 5-10 membered heteroaryl.

“Heteroarylalkynyl,” by itself or as part of another substituent refers to an acyclic alkenyl group in which one of the hydrogen atoms bonded to a carbon atom, is replaced with a heteroaryl group. In some aspects, the heteroarylalkynyl group is a 7-21 membered heteroarylalkynyl, e.g., the alkynyl moiety of the heteroarylalkynyl is (C₂-C₆) alkynyl and the heteroaryl moiety is a 5-15-membered heteroaryl. In other aspects, the heteroarylalkynyl is a 7-13 membered heteroarylalkynyl, e.g., the alkynyl moiety is (C₂-C₃) alkynyl and the heteroaryl moiety is a 5-10 membered heteroaryl.

“Heteroaryldiyl,” by itself or as part of another substituent, refers to a divalent radical derived by the removal of two hydrogen atoms from two different atoms of a parent heteroaromatic ring system, as defined herein. Typical heteroaryldiyl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some aspects, the heteroaryldiyl group comprises from 5 to 20 ring atoms (5-20 membered heteroaryldiyl). In other aspects, the heteroaryldiyl group comprises from 5 to 10 ring atoms (5-10 membered heteroaryldiyl). Exemplary heteroaryldiyl groups include those derived from furan, thiophene, pyrrole, benzothiophene, benzofuran, benzimidazole, indole, pyridine, pyrazole, quinoline, imidazole, oxazole, isoxazole and pyrazine.

“Hydrates,” refers to incorporation of water into to the form of a compound described herein, in stoichiometric proportions, resulting in the formation of an adduct. Methods of making hydrates include, but are not limited to, storage in an atmosphere containing water vapor, dosage forms that include water, or routine pharmaceutical processing steps such as, for example, crystallization (i.e., from water or mixed aqueous solvents), lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. See e.g., (Guillory, K., Chapter 5, pp. 202-205 in Polymorphism in Pharmaceutical Solids, (Brittain, H. ed.), Marcel Dekker, Inc., New York, NY, 1999). The above methods for preparing hydrates are well within the ambit of those of skill in the art, are completely conventional and do not require any experimentation beyond what is typical in the art. Hydrates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of hydrates such as, for example, HOLODIAG, Pharmaparc II, Voie de l'Innovation, 27 100 Val de Reuil, France (http://www.holodiag.com).

“N-oxide,” refers to a compound containing an N—O bond with three additional hydrogen or side chains attached to the N, or a compound containing an N—O bond with two additional hydrogen or side chains attached to the N, so that there is a positive charge on the nitrogen. The N-oxides of the present disclosure can be synthesized by oxidation procedures well known to those skilled in the art.

“Parent Aromatic Ring System,” refers to an unsaturated cyclic or polycyclic ring system having a conjugated pi electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System,” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, b-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Pharmaceutically acceptable salt,” refers to a salt of a compound which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Preventing,” or “prevention,” refers to a reduction in risk of acquiring a disease or disorder (i.e., causing at least one of the clinical symptoms of the disease not to develop in a patient that may be exposed to or predisposed to the disease but does not yet experience or display symptoms of the disease). The application of a therapeutic for preventing or prevention of a disease or disorder is known as ‘prophylaxis.’ In some aspects, the compounds provided herein provide superior prophylaxis because of lower long term side effects over long time periods.

“Prodrug,” as used herein, refers to a derivative of a drug molecule that requires a transformation within the body to release the active drug. Prodrugs are frequently, although not necessarily, pharmacologically inactive until converted to the parent drug.

“Promoiety,” as used herein, refers to a form of protecting group that when used to mask a functional group within a drug molecule converts the drug into a prodrug. Typically, the promoiety will be attached to the drug via bond(s) that are cleaved by enzymatic or non-enzymatic means in vivo.

“Protecting group,” refers to a grouping of atoms that when attached to a reactive functional group in a molecule masks, reduces or prevents reactivity of the functional group during chemical synthesis. Examples of protecting groups can be found in Green et al., “Protective Groups in Organic Chemistry,” (Wiley, 2nd ed. 1991) and Harrison et al.,

“Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Solvates,” refers to incorporation of solvents into to the crystal lattice of a compound described herein, in stoichiometric proportions, resulting in the formation of an adduct. Methods of making solvates include, but are not limited to, storage in an atmosphere containing a solvent, dosage forms that include the solvent, or routine pharmaceutical processing steps such as, for example, crystallization (i.e., from solvent or mixed solvents) vapor diffusion, etc. Solvates may also be formed, under certain circumstances, from other crystalline solvates or hydrates upon exposure to the solvent or upon suspension material in solvent. Solvates may crystallize in more than one form resulting in solvate polymorphism. See e.g., (Guillory, K., Chapter 5, pp. 202-205 in Polymorphism in Pharmaceutical Solids, (Brittain, H. ed.), Marcel Dekker, Inc., New York, NY, 1999)). The above methods for preparing solvates are well within the ambit of those of skill in the art, are completely conventional and do not require any experimentation beyond what is typical in the art. Solvates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of solvates such as, for example, HOLODIAG, Pharmaparc II, Voie de l'Innovation, 27 100 Val de Reuil, France (http://www.holodiag.com).

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include R^{a}, halo, —O—, ═O, —OR^{b}, —SR^{b}, —S—, ═S, —NR^{b}R^{c}, ═NR^{b}, ═N—OR^{b}, trihalomethyl, —CF₃, —CN, —OCN, —SCN, —NO, —NO₂, —N—OR^{b}, —N—NR^{b}R^{c}, —NR^{b}S(O)₂R^{b}, ═N₂, —N₃, —S(O)₂R^{b}, —S(O)₂NR^{b}R^{b}, —S(O)₂O—, —S(O)₂OR^{b}, —OS(O)₂R^{b}, —OS(O)₂O—, —OS(O)₂OR^{b}, —OS(O)₂NR^{b}NR^{c}, —P(O)(O—)₂, —P(O)(OR^{b})(O—), —P(O)(OR^{b})(OR^{b}), —C(O)R^{b}, —C(O)NR^{b}—OR^{b}—C(S) R^{b}, —C(NR^{b}) R^{b}, —C(O)O, —C(O)OR^{b}, —C(S)OR^{b}, —C(O)NR^{b}R^{c}, —C(NR^{b})NR^{b}R^{c}, —OC(O)R^{b}, —OC(S) R^{b}, —OC(O)O—, —OC(O)OR^{b}, —OC(O)NR^{b}R^{c}, —OC(NCN)NR^{b}R^{c}—OC(S)OR^{b}, —NR^{b}C(O)R^{b}, —NR^{b}C(S)R^{b}, —NR^{b}C(O)O, —NR^{b}C(O)OR^{b}, —NR^{b}C(NCN)OR^{b}, —NR^{b}S(O)₂NR^{b}R^{c}, —NR^{b}C(S)OR^{b}, —NR^{b}C( O)NR^{b}R^{c}, —NR^{b}C(S)NR^{b}R^{c}, —NR^{b}C(S)NR^{b}C(O)R^{a}, —NR^{b}S(O)₂OR^{b}, —NR^{b}S(O)₂R^{b}, —NR^{b}C(NCN) NR^{b}R^{c}, —NR^{b}C(NR^{b}) R^{b} and —NR^{b}C(NR^{b})NR^{b}R^{c}, where each R^{a} is independently, substituted alkyl, substituted alkenyl, substituted alkynyl, aryl, substituted aryl, cycloalkyl, substituted cycloalkyl, cycloalkenyl, substituted cycloalkenyl, heteroalkyl, substituted heteroalkyl, heteroalkenyl, substituted heteroalkenyl, heteroalkynyl, substituted heteroalkynyl, heteroaryl or substituted heteroaryl; each R^{b} is independently hydrogen, substituted alkyl, substituted alkenyl, substituted alkynyl, cycloalkyl, substituted cycloalkyl, cycloalkenyl, substituted cycloalkenyl, heteroalkyl, substituted heteroalkyl, heteroalkenyl, substituted heteroalkenyl, heteroalkynyl, substituted heteroalkynyl, arylalkyl, substituted arylalkyl, arylalkenyl, substituted arylalkenyl, arylalkynyl, substituted arylalkynyl, heteroarylalkyl, substituted heteroarylalkyl, heteroarylalkenyl, substituted heteroarylalkenyl, heteroarylalkynyl or substituted heteroarylalkynyl; and each R^{c} is independently R^{b} or alternatively, the two R^{c}s taken together with the nitrogen atom to which they are bonded form a 4-, 5-, 6- or 7 membered-cycloheteroalkyl, substituted cycloheteroalkyl, cycloheteroalkenyl, substituted cycloheteroalkenyl ring or a cycloheteroalkyl or cycloheteroalkenyl fused with an aryl group which may optionally include from 1 to 4 of the same or different additional heteroatoms selected from the group consisting of O, N and S. As specific examples, —NR^{b}R^{c} is meant to include —NH₂, —NH-alkyl, N-pyrrolidinyl and N-morpholinyl. In other aspects, substituent groups useful for substituting saturated carbon atoms in the specified group or radical include R^{a}, halo, —OR^{b}, —NR^{b}R^{c}, trihalomethyl, —CN, —NR^{b}S(O)₂R^{b}, —C(O)R^{b}, —C(O)NR^{b}—OR^{b}, —C(O)OR^{b}, —C(O)NR^{b}R^{c}, —OC(O)R^{b}, —OC(O)OR^{b}, —OS(O)₂NR^{b}NR^{c}, —OC(O)NR^{b}R^{c}, and —NR^{b}C(O)OR^{b}, where R^{a}, R^{b} and R^{c} are as previously defined. In still other aspects, substituent groups useful for substituting saturated carbon atoms in the specified group or radical include R^{a}, halo, —OR^{b}, —NR^{b}R^{c}, trihalomethyl, —CN, —C(O)R^{b}, —C(O)OR^{b}, —C(O)NR^{b}R^{c}, —OC(O)R^{b}, —OC(O)NR^{b}R^{c}, and —NR^{b}C(O)OR^{b},where R^{a}, R^{b} and R^{c} are as previously defined.

Substituent groups useful for substituting unsaturated carbon atoms in the specified group or radical include substituted alkyl, —R^{a}, halo, —O—, —OR^{b}, —SR^{b}, —S—, —NR^{b}R^{c}, trihalomethyl, —CF₃, —CN, —OCN, —SCN, —NO, —NO₂, —N₃, —S(O)₂₀, —S(O)₂OR^{b}, —OS(O)₂R^{b}, —OS (0)₂OR^{b}, —OS(O)₂₀, —P(O)(O—)₂, —P(O)(OR^{b})(O—), —P(O)(OR^{b})(OR^{b}), —C(O)R^{b}, —C(S)R^{b}, —C(NR^{b}) R^{b}, —C(O)O—, —C(O)OR^{b}, —C(S)OR^{b}, —C(O)NR^{b}R^{c}, —C(NR^{b})NR^{b}R^{c}, —OC(O)R^{b}, —OC(S)R^{b}, —OC(O) O-, —OC(O)OR^{b}, —OC(S)OR^{b}, —OC(O)NR^{b}R^{c}, —OS(O)₂NR^{c}NR^{c}, —NR^{b}C(O)R^{b}, —NR^{b}C(S)R^{b}, —NR^{b} C(O)O—, —NR^{b}C(O)OR^{b}, —NR^{b}S(O)₂OR^{a}, —NR^{b}S(O)₂R^{a}, —NR^{b}C(S)OR^{b}, —NR^{b}C(O)NR^{b}R^{c}, —NR^{b}C(NR^{b}) R^{b}, —NR^{b}C(NR^{b})NR^{b}R^{c} and —C(NR^{b})NR^{b}C(NR^{b})NR^{b}R^{c} where R^{a}, R^{b} and R^{c} are as previously defined. In other aspects, substituent groups useful for substituting unsaturated carbon atoms in the specified group or radical include substituted alkyl, —R^{a}, halo, —OR^{b}, —SR^{b}, —NR^{b}R^{c}, trihalomethyl, —CN, —S(O)₂OR^{b}, —C(O)R^{b}, —C(O)OR^{b}, —C(O)NR^{b}R^{c}, —OC(O)R^{b}, —OC(O)OR^{b}, —OS(O) 2NR^{b}NR^{c}, —NR^{b}C(O)R^{b} and —NR^{b}C(O)OR^{b}, where R^{a}, R^{b} and R^{c} are as previously defined. In still other aspects, substituent groups useful for substituting unsaturated carbon atoms in the specified group or radical include substituted alkyl, —R^{a}, halo, —OR^{b}, —NR^{b}R^{c}, trihalomethyl, —S(O)₂OR^{b}, —C(O)R^{b}, —C(O)OR^{b}, —C(O)NR^{b}R^{c}, —OC(O)R^{b}, —NR° C. (O)R^{b} and —NR^{b}C(O)OR^{b}, where R^{a}, R^{b} and R^{c} are as previously defined.

Substituent groups useful for substituting nitrogen atoms in heteroalkyl and cycloheteroalkyl groups include, but are not limited to, alkyl, —R^{a}, —O—, —OR^{b}, —SR^{b}, —S—, —NR^{b}R^{c}, trihalomethyl, —CF₃, —CN, —NO, —NO₂, —S(O)₂R^{b}, —S(O)₂O—, —S(O)₂OR^{b}, —OS(O)₂R^{b}, —OS(O)₂O—, —OS(O)₂OR^{b}, —P(O)(O—)₂, —P(O)(OR^{b})(O—), —P(O)(OR^{b})(OR^{b}), —C(O)R^{b}, —C(S)R^{b}, —C(NR^{b}) R^{b}, —C(O) OR^{b}, —C(S)OR^{b}, —C(O)NR^{b}R^{c}, —C(NR^{b})NR^{b}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{b}R^{c}, —NR^{b}C(NR^{b}) R^{b}, —NR^{b}C(NR^{b})NR^{b}R^{c} and —C(NR^{b})NR^{b}C(NR^{b})NR^{b}R^{c} where R^{a}, R^{b} and R^{c} are as previously defined. In some aspects, substituent groups useful for substituting nitrogen atoms in heteroalkyl and cycloheteroalkyl groups include, alkyl, R^{a}, halo, —OR^{b}, —NR^{b}R^{c}, trihalomethyl, —CN, —S(O)₂OR^{b}, —OS(O)₂R^{b}, —C(O)R^{b}, —C(NR^{b}) R^{b}, —C(O)OR^{b}, —C(O)NR^{b}R^{c}, —OC(O)R^{b}, —OC(O)OR^{b}, —OS(O)₂NR^{c}NR^{c}, —NR^{b}C(O)R^{b} and —NR^{b}C(O)OR^{b}, where R^{a}, R^{b} and R^{c} are as previously defined. In still other aspects, substituent groups useful for substituting nitrogen atoms in heteroalkyl and cycloheteroalkyl groups include, alkyl, R^{a}, halo, —OR^{b}, —NR^{b}R^{c}, trihalomethyl, —CN, —S(O)₂OR^{b}, —C(O)R^{b}, —C(NR^{b}) R^{b}, —C(O)OR^{b}, —C(O)NR^{b}R^{c}, —OC(O)R^{b}, —NR^{b} C(O)R^{b} and —NR^{b}C(O)OR^{b}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Subject,” “individual,” or “patient,” is used interchangeably herein and refers to a vertebrate, preferably a mammal. Mammals include, but are not limited to, rodents, simians, humans, farm animals, sport animals and pets. In some aspects, the subject, individual, or patient is a member of the species Homo sapiens. In other aspects, the subject, individual, or patient includes all mammals except Homo sapiens.

“Treating,” or “treatment,” of any disease or disorder refers, in some aspects, to ameliorating the disease or disorder (i.e., arresting or reducing the development of the disease or at least one of the clinical symptoms thereof). Treatment may also be considered to include preemptive or prophylactic administration to ameliorate, arrest or prevent the development of the disease or at least one of the clinical symptoms. In a further feature the treatment rendered has lower potential for long-term side effects over multiple years. In other aspects “treating” or “treatment” refers to ameliorating at least one physical parameter, which may not be discernible by the patient. In yet other aspects, “treating” or “treatment” refers to inhibiting the disease or disorder, either physically (e.g., stabilization of a discernible symptom), physiologically (e.g., stabilization of a physical parameter) or both. In yet other aspects, “treating” or “treatment” refers to delaying the onset of the disease or disorder.

“Therapeutically effective amount,” means the amount of a compound that, when administered to a patient for treating a disease, is sufficient to treat the disease. The “therapeutically effective amount” will vary depending on the compound, the disease and its severity and the age, weight, adsorption, distribution, metabolism and excretion etc., of the patient to be treated.

“Vehicle,” refers to a diluent, excipient or carrier with which a compound is administered to a subject. In some aspects, the vehicle is pharmaceutically acceptable.

### Compounds

In one aspect, a compound of structure:

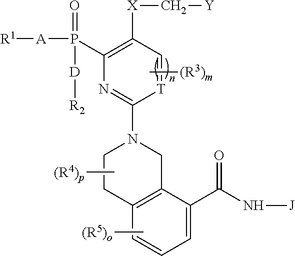

or salts, hydrates and solvates thereof is provided wherein, X is heteroaryl or substituted heteroaryl; Y is alkyl, substituted alkyl, cycloalkyl, substituted cycloalkyl, heteroalkyl or substituted heteroalkyl; A is —O—or —NR⁶—; D is —O—or absent; J is heteroaryl or substituted heteroaryl; R¹, R² and R⁶ are independently-H, alkyl, alkenyl, aryl, substituted aryl, arylalkyl, substituted arylalkyl, cycloalkyl, substituted cycloalkyl, cycloalkenyl or substituted cycloalkenyl wherein substituted arylalkyl includes substitution with a carbohydrate derivative; each of R³ and R⁵ are independently halo, alkyl, alkenyl, alkynyl or haloalkyl; R⁴ is halo, alkyl, alkenyl, alkynyl or haloalkyl or alternatively two R⁴ attached to the carbon atom form a cycloalkyl or cycloalkenyl ring; m is 0, 1, 2 or 3; n is 0 or 1; o is 0, 1, 2 or 3; and p is 0, 1, 2 or 3.

In some embodiments, n is 1 and T is —C—. In other embodiments, n is 0 and T is —S—.

In some embodiments, X is substituted heteroaryl. In other embodiments, Y is cycloalkyl or substituted alkyl. In still other embodiments, A is —O—. In still other embodiments, R¹ is —H, alkyl or substituted arylalkyl. In still other embodiments, D is absent. In still other embodiments, J is heteroaryl. In still other embodiments, m, p and o are 0.

In some embodiments, n is 1 and T is —C—, X is substituted heteroaryl, Y is cycloalkyl, A is —O—, R¹ is —H, alkyl or substituted arylalkyl, D is alkyl, J is heteroaryl and m, o and p are 0. In other embodiments, compounds having the structure

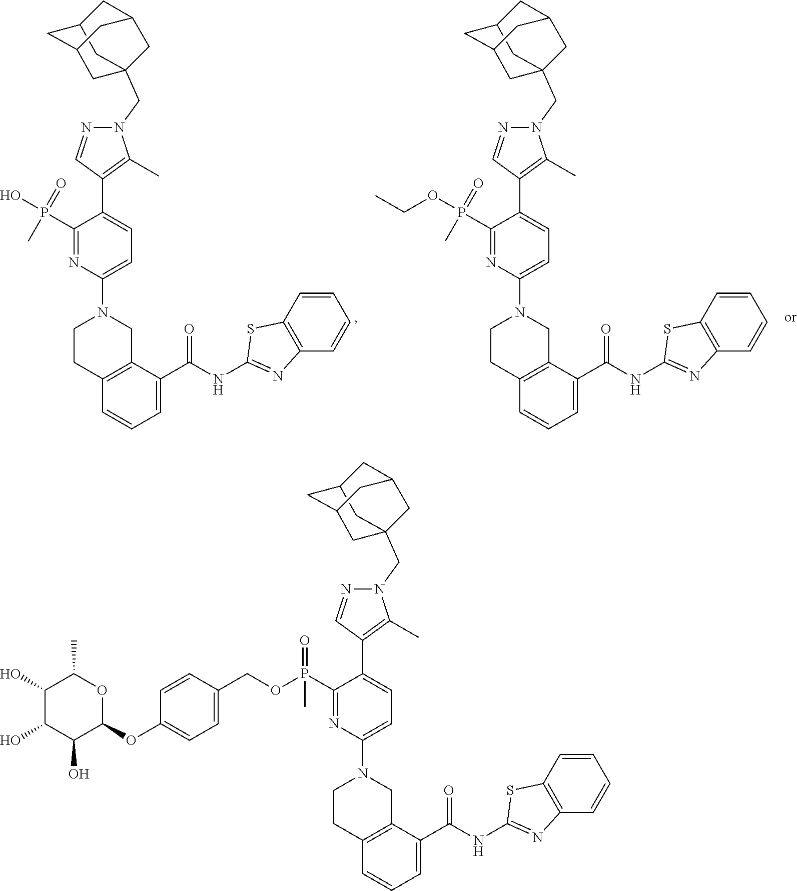 [SMILES: Cc1c(-c2ccc(N3CCc4cccc(C(=O)Nc5nc6ccccc6s5)c4C3)nc2P(C)(=O)O)cnn1CC12CC3CC(CC(C3)C1)C2 ; CCOP(C)(=O)c1nc(N2CCc3cccc(C(=O)Nc4nc5ccccc5s4)c3C2)ccc1-c1cnn(CC23CC4CC(CC(C4)C2)C3)c1C ; Cc1c(-c2ccc(N3CCc4cccc(C(=O)Nc5nc6ccccc6s5)c4C3)nc2P(C)(=O)OCc2ccc(O[C@@H]3O[C@@H](C)[C@@H](O)[C@@H](O)[C@@H]3O)cc2)cnn1CC12CC3CC(CC(C3)C1)C2]

are provided.

In some embodiments, n is 0 and T is —S—, X is substituted heteroaryl, Y is substituted alkyl, A is —O—, R¹ is —H or alkyl, D is alkyl, J is heteroaryl and m, o and p are 0. In other embodiments, a compound having the structure:

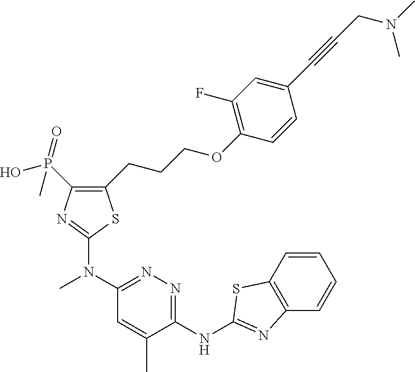 [SMILES: Cc1cc(N(C)c2nc(P(C)(=O)O)c(CCCOc3ccc(C#CCN(C)C)cc3F)s2)nnc1Nc1nc2ccccc2s1]

In one aspect, a compound of the structure below is provided:

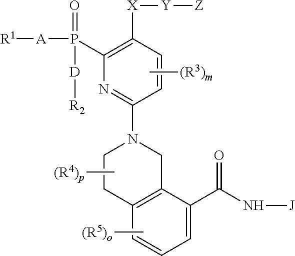

or salts, hydrates and solvates thereof where X is heteroaryl or substituted heteroaryl; Y is alkyldiyl, substituted alkyldiyl, heteroalkyldiyl or substituted heteroalkyldiyl; Z is cycloalkyl, substituted cycloalkyl, cycloalkenyl, substituted cycloalkenyl, aryl, substituted aryl, cycloheteroalkyl or substituted cycloheteroalkyl; A is —O—or —NR⁶—; D is —O—or absent; J is heteroaryl or substituted heteroaryl; R¹, R² and R⁶ are independently-H alkyl, alkenyl, aryl, substituted aryl, arylalkyl, substituted arylalkyl cycloalkyl, substituted cycloalkyl, cycloalkenyl or substituted cycloalkenyl wherein substituted arylalkyl includes substitution with a carbohydrate derivative; each R³ and R⁵ are independently halo, alkyl, alkenyl, alkynyl or haloalkyl; R⁴ is halo, alkyl, alkenyl, alkynyl or haloalkyl or alternatively two R⁴ attached to the carbon atom form a cycloalkyl or cycloalkenyl ring; m is 0, 1, 2 or 3, n is 0, 1, 2, 3, 4, 5 or 6; and o is 0, 1, 2 or 3.

In some embodiments, X is substituted heteroaryl. In other embodiments, Y is alkyldiyl.

In some embodiments, Z is cycloalkyl, substituted cycloalkyl, cycloalkenyl or substituted cycloalkenyl. In other embodiments, Z is cycloalkyl.

In some embodiments, A is —O—. In other embodiments, R¹ is —H, alkyl, or substituted arylalkyl where substituted arylalkyl includes substitution substituted with a carbohydrate derivative. In still other embodiments, D is absent. In still other embodiments, J is heteroaryl. In still other embodiments, m, n and o are 0.

In some embodiments, X is substituted heteroaryl, Y is alkyldiyl, Z is cycloalkyl, A is —O—, R¹ is —H, alkyl, or substituted arylalkyl where substituted arylalkyl includes substitution substituted with a carbohydrate derivative, D is alkyl, J is heteroaryl and m, n and o are 0.

In another aspect a compound of structure:

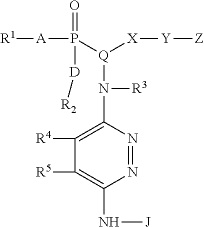

or salts, hydrates and solvates thereof where Q is heteroaryl or substituted heteroaryl; X is alkyldiyl, substituted alkyldiyl, heteroalkyldiyl or substituted heteroalkyldiyl; Y is —O—, —NR⁶—, or —CH₂—; Z is cycloalkyl, substituted cycloalkyl, cycloalkenyl, substituted cycloalkenyl, aryl, substituted aryl, cycloheteroalkyl, substituted cycloheteroalkyl heteroaryl or substituted heteroaryl; A is —O—or —NR⁷—; D is —O—or absent; J is heteroaryl or substituted heteroaryl; R¹, R², R³ and R⁷ are independently-H, alkyl, alkenyl, aryl, substituted aryl, arylalkyl or substituted arylalkyl where substituted arylalkyl includes substitution with a carbohydrate derivative; R⁴ and R⁵ are independently-H, alkyl, substituted alkyl, alkenyl, substituted alkenyl, cycloalkyl, substituted cycloalkyl or together with the atoms to which they are bonded form a cycloalkenyl or substituted cycloalkenyl group; and R⁶ is —H or alkyl is provided.

In some embodiments Q is heteroaryl. In other embodiments, X is alkyldiyl. In still other embodiments, Y is —O—. In still other embodiments, Z is substituted aryl.

In some embodiments, A is —O—. In other embodiments, D is absent. In still other embodiments, R¹ is —H, R² and R³ are independently alkyl. In still other embodiments, R⁴ is —H and R⁵ is alkyl. In still other embodiments, J is heteroaryl.

In some embodiments, Q is heteroaryl, X is alkyldiyl, Y is —O, Z is substituted aryl, A is —O—, D is absent, R¹ is —H, R² and R³ are independently alkyl, R⁴ is —H, R⁵ is alkyl and J is heteroaryl.

The compounds above can be made by well-known procedures some of which are exemplified in the experimental section.

### Methods for Characterizing and Identifying Senolytic Agents

Characterizing a senolytic agent can be determined using one or more cell-based assays and one or more animal models described herein or in the art and with which a person skilled in the art will be familiar. A senolytic agent may selectively kill one or more types of senescent cells (e.g., senescent preadipocytes, senescent endothelial cells, senescent fibroblasts, senescent neurons, senescent epithelial cells, senescent mesenchymal cells, senescent smooth muscle cells, senescent macrophages, or senescent chondrocytes). In certain embodiments, a senolytic agent is capable of selectively killing at least senescent fibroblasts.

Characterizing an agent as a senolytic agent can be accomplished using one or more cell-based assays and one or more animal models described herein or in the art. Those of skill in the art will readily appreciate that characterizing an agent as a senolytic agent and determining the level of killing by an agent can be accomplished by comparing the activity of a test agent with appropriate negative controls (e.g., vehicle or diluent only and/or a composition or compound known in the art not to kill senescent cells) and appropriate positive controls. In vitro cell-based assays for characterizing senolytic agents also include controls for determining the effect of the agent on non-senescent cells (e.g., quiescent cells or proliferating cells). A senolytic agent reduces (i.e., decreases) percent survival of a plurality of senescent cells (i.e., in some manner reduces the quantity of viable senescent cells in the animal or in the cell-based assay) compared with one or more negative controls. Conditions for a particular in vitro assay include temperature, buffers (including salts, cations, media), and other components, which maintain the integrity of the test agent and reagents used in the assay, are familiar to a person skilled in the art and/or which can be readily determined through routine experimentation.

The source of senescent cells for use in assays may be a primary cell culture, or culture-adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiable cell lines, transformed cell lines, and the like. In some embodiments, senescent cells are isolated from biological samples obtained from a host or subject who has a senescent cell associated disease or disorder. In other embodiments, non-senescent cells may be obtained from a subject or may be a culture adapted line and senescence is induced by methods described herein and, in the art, such as by exposure to irradiation or a chemotherapeutic agent (e.g., doxorubicin). Biological samples may be, for example, blood samples, biopsy specimens, body fluids (e.g., lung lavage, ascites, mucosal washings, synovial fluid, etc.), bone marrow, lymph nodes, tissue explants, organ cultures, or any other tissues or cell preparations obtained from a subject. The biological samples may be a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. The subject may be a human or non-human animal.

Transgenic animal models as described herein and, in the art, may be used to determine killing or removal of senescent cells (see, e.g., Baker et al., Nature, 479 (2011)₂₃₂-236; International Application No. WO/2012/177927; International Application No. WO 2013/090645). Exemplary transgenic animal models contain a transgene that includes a nucleic acid that allows for controlled clearance of senescent cells (e.g., p16INK4a positive senescent cells) as a positive control. The presence and level of senescent cells in the transgenic animals can be determined by measuring the level of a detectable label or labels that are expressed in senescent cells of the animal. The transgene nucleotide sequence includes a detectable label, for example, one or more of a red fluorescent protein; a green fluorescent protein; and one or more luciferases to detect clearance of senescent cells.

Animal models that are described herein or in the art include art-accepted models for determining the effectiveness of a senolytic agent to treat or prevent (i.e., reduce the likelihood of occurrence of) a particular senescence associated disease or disorder, such as atherosclerosis models, osteoarthritis models, COPD models, IPF models, etc. As described herein, pulmonary disease murine models, such as a bleomycin pulmonary fibrosis model, and a chronic cigarette smoking model are applicable for diseases such as COPD and may be routinely practiced by a person skilled in the art. Animal models for determining the effectiveness of a senolytic agent to treat and/or prevent (i.e., reduce the likelihood of occurrence of) chemotherapy and radiotherapy side effect models or to treat or prevent (i.e., reduce the likelihood of occurrence of) metastasis are described in International Application Nos. WO 2013/090645 and WO 2014/205244. Animal models for determining the effectiveness of agents for treating eye diseases, particularly age-related macular degeneration is also routinely used in the art (see, e.g., Pennesi et al.; Mol. Aspects Med. 33 (2012) 487-509; Zeiss et al., Vet. Pathol. 47 (2010) 396-413; Chavala et al., J. Clin. Invest. 123 (2013) 4170-4181).

By way of non-limiting example and as described herein, osteoarthritis animal models have been developed. Osteoarthritis may be induced in the animal, for example, by inducing damage to a joint, for example, in the knee by surgical severing, incomplete or total, of the anterior cruciate ligament. Osteoarthritis animal models may be used for assessing the effectiveness of a senolytic agent to treat or prevent (i.e., reducing the likelihood of occurrence of) osteoarthritis and cause a decrease in proteoglycan erosion and to induce (i.e., stimulate, enhance) collagen (such as collagen type 2) production, and to reduce pain in an animal that has ACL surgery. Immunohistology may be performed to examine the integrity and composition of tissues and cells in a joint. Immunochemistry and/or molecular biology techniques may also be performed, such as assays for determining the level of inflammatory molecules (e.g., IL-6) and assays for determining the level of senescence markers as noted above, using methods and techniques described herein, which may be routinely practiced by a person skilled in the art.

By way of another non-limiting example and as described herein, atherosclerosis animal models have been developed. Atherosclerosis may be induced in the animal, for example, by feeding animals a high fat diet or by using transgenic animals highly susceptible to developing atherosclerosis. Animal models may be used for determining the effectiveness of a senolytic agent to reduce the amount of plaque or to inhibit formation of plaque in an atherosclerotic artery, to reduce the lipid content of an atherosclerotic plaque (i.e., reduce, decrease the amount of lipid in a plaque), and to cause an increase or to enhance fibrous cap thickness of a plaque. Sudan staining may be used to detect the level of lipid in an atherosclerotic vessel. Immunohistology and immunochemistry and molecular biology assays (e.g., for determining the level of inflammatory molecules (e.g., IL-6), and for determining the level of senescence markers as noted above), may all be performed according to methods described herein, which are routinely practiced in the art.

In still another non-limiting example, and as described herein, mouse models in which animals are treated with bleomycin have been described (see, e.g., Peng et al., PLOS One 8 (4) (2013) e59348. doi: 10.1371/journal.pone.0059348; Mouratis et al., Curr. Opin. Pulm. Med. 17 (2011) 355-361) for determining the effectiveness of an agent for treating IPF. In pulmonary disease animal models (e.g., a bleomycin animal model, smoke-exposure animal model, or the like), respiratory measurements may be taken to determine elastance, compliance, static compliance, and peripheral capillary oxygen saturation (SpO₂). Immunohistology and immunochemistry and molecular biology assays (e.g., for determining the level of inflammatory molecules (e.g., IL-6), and for determining the level of senescence markers as noted above), may all be performed according to methods described herein, which are routinely practiced in the art.

Determining the effectiveness of a senolytic agent to selectively kill senescent cells as described herein in an animal model may be performed using one or more statistical analyses with which those skilled in the art will be familiar. By way of example, statistical analyses such as two-way analysis of variance (ANOVA) may be used for determining the statistical significance of differences between animal groups treated with an agent and those that are not treated with the agent (i.e., negative control group, which may include vehicle only and/or a non-senolytic agent). Statistical packages such as SPSS, MINITAB, SAS, Statistika, Graphpad, GLIM, Genstat, and BMDP are readily available and are routinely used by a person skilled in the animal model art.

Those of skill in the art will readily appreciate that characterizing a senolytic agent and determining the level of killing by the senolytic agent can be accomplished by comparing the activity of a test agent with appropriate negative controls (e.g., vehicle only and/or a composition, agent, or compound known in the art not to kill senescent cells) and appropriate positive controls. In vitro cell-based assays for characterizing the agent also include controls for determining the effect of the agent on non-senescent cells (e.g., quiescent cells or proliferating cells). A senolytic agent that is useful reduces (i.e., decreases) percent survival of senescent cells (i.e., in some manner reduces the quantity of viable senescent cells in the animal or in the cell-based assay) compared with one or more negative controls. Accordingly, a senolytic agent selectively kills senescent cells compared with killing of non-senescent cells (which may be referred to herein as selectively killing senescent cells over non-senescent cells).

In certain embodiments (either in an in vitro assay or in vivo (in a human or non-human animal)), the at least one senolytic agent kills at least 20% of the senescent cells and kills no more than 5% of non-senescent cells. In other embodiments (either in an in vitro assay or in vivo (in a human or non-human animal)), the at least one senolytic agent kills at least about 25%, 30%, 35%, 40%, 45%, 50%, 55%, 60%, or 65% of the senescent cells and kills no more than about 5% or 10% of non-senescent cells. In still other embodiments (either in an in vitro assay or in vivo (in a human or non-human animal)), the at least one senolytic agent kills at least about 30%, 35%, 40%, 45%, 50%, 55%, 60%, or 65% of the senescent cells and kills no more than about 5%, 10%, or 15% of non-senescent cells. In still other embodiments (either in an in vitro assay or in vivo (in a human or non-human animal)), the at least one senolytic agent kills at least about 40%, 45%, 50%, 55%, 60%, or 65% of the senescent cells and kills no more than about 5%, 10%, 15%, 20%, or 25% of non-senescent cells. In still other embodiments (either in an in vitro assay or in vivo (in a human or non-human animal)), the at least one senolytic agent kills at least about 50%, 55%, 60%, or 65% of the senescent cells and kills no more than about 5%, 10%, 15%, 20%, 25%, or 30% of non-senescent cells. Stated another way, a senolytic agent has at least 5-25, 10-50, 10-100 or 100-1000 times greater selectively for killing senescent cells than for non-senescent cells.

With respect to specific embodiments of the methods described herein for treating a senescence-associated disease or disorder, the percent senescent cells killed may refer to the percent senescent cells killed in a tissue or organ that comprises senescent cells that contribute to onset, progression, and/or exacerbation of the disease or disorder. By way of non-limiting example, tissues of the brain, tissues and parts of the eye, pulmonary tissue, cardiac tissue, arteries, joints, skin, and muscles may comprise senescent cells that may be reduced in percent as described above by the senolytic agents described herein and thereby provide a therapeutic effect. Moreover, selectively removing at least 20% or at least 25% of senescent cells from an affected tissue or organ can have a clinically significant therapeutic effect.

With respect to specific embodiments of the methods described herein, such as for example, treating a cardiovascular disease or disorder associated with arteriosclerosis, such as atherosclerosis, by administering a senolytic agent (i.e., in reference to vivo methods above), the percent senescent cells killed may refer to the percent senescent cells killed in an affected artery containing plaque versus non-senescent cells killed in the arterial plaque. In certain embodiments, in the methods for treating the cardiovascular disease, such as atherosclerosis, as described herein, the at least one senolytic agent kills at least 20% of the senescent cells and kills no more than 5% of non-senescent cells in the artery. In other embodiments, the senolytic agent selectively kills at least 25% of the senescent cells in the arteriosclerotic artery.

In some embodiments, with respect to the methods described herein for treating osteoarthritis by administering a senolytic agent, the percent senescent cells killed may refer to the percent senescent cells killed in an osteoarthritic joint versus non-senescent cells killed in the osteoarthritic joint. In certain embodiments, in the methods for treating osteoarthritis as described herein, the at least one senolytic agent kills at least 20% of the senescent cells and kills no more than 5% of non-senescent cells in the osteoarthritic joint. In other embodiments, the senolytic agent selectively kills at least 25% of the senescent cells in the osteoarthritic joint.

In some embodiments, with respect to the methods described herein for treating senescence associated pulmonary disease or disorder (e.g., COPD, IPF) by administering at least one senolytic agent, the percent senescent cells killed may refer to the percent senescent cells killed in affected pulmonary tissue versus non-senescent cells killed in the affected pulmonary tissue of the lung. In certain embodiments, in the methods for treating senescence associated pulmonary diseases and disorders as described herein, a senolytic agent kills at least 20% of the senescent cells and kills no more than 5% of non-senescent cells in the affected pulmonary tissue. In other embodiments, the senolytic agent selectively kills at least 25% of the senescent cells in the affected pulmonary tissue.

In certain embodiments, methods are provided for identifying (i.e., screening for) agents that are useful senolytic agents for treating or preventing (i.e., reducing the likelihood of occurrence of) a senescence associated disease or disorder. In some embodiments, a method for identifying a senolytic agent for treating such diseases and disorders comprises inducing cells to senesce to provide established senescent cells. Methods for inducing cells to senesce are described herein and in the art and include, for example, exposure to radiation (e.g., 10 Gy is typically sufficient) or a chemotherapeutic agent (e.g., doxorubicin or other anthracyclines). After exposure to the agent, the cells are cultured for an appropriate time and under appropriate conditions (e.g., media, temperature, CO₂/O₂ level appropriate for a given cell type or cell line) to allow senescence to be established. As discussed herein, senescence of cells may be determined by determining any number of characteristics, such as changes in morphology (as viewed by microscopy, for example); production of, for example, senescence-associated-galactosidase (SA-gal), p16INK4a, p21, or any one or more SASP factors (e.g., IL-6, MMP3). A sample of the senescent cells is then contacted with a candidate agent (i.e., mixed with, combined, or in some manner permitting the cells and the agent to interact). Persons skilled in the art will appreciate that the assay will include the appropriate controls, negative and positive, either historical or performed concurrently. For example, a sample of control non-senescent cells that have been cultured similarly as the senescent cells but not exposed to a senescence inducing agent are contacted with the candidate agent. The level of survival of the senescent cells is determined and compared with the level of survival of the non-senescent cells. A senolytic agent is identified when the level of survival of the senescent cells is less than the level of survival of the non-senescent cells.

In some embodiments, the above-described method to identify a senolytic agent may further comprise steps for identifying whether the senolytic agent is useful for treating osteoarthritis. The method may further comprise contacting the identified senolytic agent with cells capable of producing collagen; and determining the level of collagen produced by the cells. In some embodiments, the cells are chondrocytes and the collagen is Type 2 collagen. The method may further comprise administering a candidate senolytic agent to a non-human animal with arthritic lesions in a joint and determining one or more of (a) the level of senescent cells in the joint; (b) physical function of the animal; (c) the level of one or more markers of inflammation; (d) histology of the joint; and (e) the level of Type 2 collagen produced, thereby determining therapeutic efficacy of the senolytic agent wherein one or more of the following is observed in the treated animal compared with an animal not treated with the senolytic agent: (i) a decrease in the level of senescent cells in the joint of the treated animal; (ii) improved physical function of the treated animal; (iii) a decrease in the level of one or more markers of inflammation in the treated animal; (iv) increased histological normalcy in the joint of the treated animal; and (v) an increase in the level of Type 2 collagen produced in the treated animal. As described herein and in the art, the physical function of the animal may be determined by techniques that determine the sensitivity of a leg to an induced or natural osteoarthritic condition, for example, by the animal's tolerance to bear weight on an affected limb or the ability of the animal to move away from an unpleasant stimulus, such as heat or cold. Determining the effectiveness of an agent to kill senescent cells as described herein in an animal model may be performed using one or more statistical analyses with which a skilled person will be familiar. Statistical analyses as described herein and routinely practiced in the art may be applied to analyze data.

In other embodiments, the above-described method to identify a senolytic agent may further comprise steps for identifying whether the senolytic agent is useful for treating a cardiovascular disease caused by or associated with arteriosclerosis. Accordingly, the method may further comprise administering the senolytic candidate agent in non-human animals or in animal models for determining the effectiveness of an agent to reduce the amount of plaque, to inhibit formation of plaque in an atherosclerotic artery, to reduce the lipid content of an atherosclerotic plaque (i.e., reduce, decrease the amount of lipid in a plaque), and/or to cause an increase or to enhance fibrous cap thickness of a plaque. Sudan staining may be used to detect the level of lipid in an atherosclerotic vessel. Immunohistology, assays for determining the level of inflammatory molecules (e.g., IL-6), and/or assays for determining the level of senescence markers as noted above, may all be performed according to methods described herein and routinely practiced in the art.

In a specific embodiment, methods described herein for identifying a senolytic agent may further comprise administering a candidate senolytic agent to a non-human animal with atherosclerotic plaque and determining one or more of (a) the level of senescent cells in the artery; (b) physical function of the animal; (c) the level of one or more markers of inflammation; (d) histology of the affected blood vessel(s) (e.g., artery); and thereby determining therapeutic efficacy of the senolytic agent wherein one or more of the following is observed in the treated animal compared with an animal not treated with the senolytic agent: (i) a decrease in the level of senescent cells in the artery of the treated animal; (ii) improved physical function of the treated animal; (iii) a decrease in the level of one or more markers of inflammation in the treated animal; (iv) increased histological normalcy in the artery of the treated animal. As described herein and in the art, the physical function of the animal may be determined by measuring physical activity. Statistical analyses as described herein and routinely practiced in the art may be applied to analyze data.

In some embodiments, methods described herein for identifying a senolytic agent may comprise administering a candidate senolytic agent to a non-human animal pulmonary disease model such as a bleomycin model or a smoke-exposure animal model and determining one or more of (a) the level of senescent cells in a lung; (b) lung function of the animal; (c) the level of one or more markers of inflammation; (d) histology of pulmonary tissue, thereby determining therapeutic efficacy of the senolytic agent wherein one or more of the following is observed in the treated animal compared with an animal not treated with the senolytic agent: (i) a decrease in the level of senescent cells in the lungs and pulmonary tissue of the treated animal; (ii) improved lung function of the treated animal; (iii) a decrease in the level of one or more markers of inflammation in the treated animal; and (iv) increased histological normalcy in the pulmonary tissue of the treated animal. Respiratory measurements may be taken to determine elastance, compliance, static compliance, and peripheral capillary oxygen saturation (SpO₂). Lung function may be evaluated by determining any one of numerous measurements, such as expiratory reserve volume (ERV), forced vital capacity (FVC), forced expiratory volume (FEV) (e.g., FEV in one second, FEV1), FEV1/FEV ratio, forced expiratory flow 25% to 75%, and maximum voluntary ventilation (MVVpeak expiratory flow (PEF), slow vital capacity (SVC). Total lung volumes include total lung capacity (TLC), vital capacity (VC),), residual volume (RV), and functional residual capacity (FRC). Gas exchange across alveolar capillary membrane can be measured using diffusion capacity for carbon monoxide (DLCO). Peripheral capillary oxygen saturation (SpO.sub.2) can also be measured. Statistical analyses as described herein and routinely practiced in the art may be applied to analyze data.

### Methods of Treatment and Prevention of Senescence-Associated Diseases and Disorders

Methods are provided herein for treating conditions, diseases, or disorders related to, associated with, or caused by cellular senescence, including age-related diseases and disorders in a subject in need thereof. A senescence-associated disease or disorder may also be called herein a senescent cell-associated disease or disorder. Senescence-associated diseases and disorders include, for example, cardiovascular diseases and disorders, inflammatory diseases and disorders, autoimmune diseases and disorders, pulmonary diseases and disorders, eye diseases and disorders, metabolic diseases and disorders, neurological diseases and disorders (e.g., neurodegenerative diseases and disorders); age-related diseases and disorders induced by senescence; skin conditions; age-related diseases; dermatological diseases and disorders; and transplant related diseases and disorders. A prominent feature of aging is a gradual loss of function, or degeneration that occurs at the molecular, cellular, tissue, and organismal levels. Age-related degeneration gives rise to well-recognized pathologies, such as sarcopenia, atherosclerosis and heart failure, osteoporosis, pulmonary insufficiency, renal failure, neurodegeneration (including macular degeneration, Alzheimer's disease, and Parkinson's disease), and many others. Although different mammalian species vary in their susceptibilities to specific age-related pathologies, collectively, age-related pathologies generally rise with approximately exponential kinetics beginning at about the mid-point of the species-specific life span (e.g., 50-60 years of age for humans) (see, e.g., Campisi, Annu. Rev. Physiol. 75 (2013) 685-705; Naylor et al., Clin. Pharmacol. Ther. 93 (2013) 105-116).

Examples of senescence-associated conditions, disorders, or diseases that may be treated by administering any one of the senolytic agents described herein according to the methods described herein include, cognitive diseases (e.g., mild cognitive impairment (MCI), Alzheimer's disease and other dementias; Huntington's disease); cardiovascular disease (e.g., atherosclerosis, cardiac diastolic dysfunction, aortic aneurysm, angina, arrhythmia, cardiomyopathy, congestive heart failure, coronary artery disease, myocardial infarction, endocarditis, hypertension, carotid artery disease, peripheral vascular diseases, cardiac stress resistance, cardiac fibrosis); metabolic diseases and disorders (e.g., obesity, diabetes, metabolic syndrome); motor function diseases and disorders (e.g., Parkinson's disease, motor neuron dysfunction (MND); Huntington's disease); cerebrovascular disease; emphysema; osteoarthritis; benign prostatic hypertrophy; pulmonary diseases (e.g., idiopathic pulmonary fibrosis, chronic obstructive pulmonary disease (COPD), emphysema, obstructive bronchiolitis, asthma); inflammatory/autoimmune diseases and disorders (e.g., osteoarthritis, eczema, psoriasis, osteoporosis, mucositis, transplantation related diseases and disorders); ophthalmic diseases or disorders (e.g., age-related macular degeneration, cataracts, glaucoma, vision loss, presbyopia); diabetic ulcer; metastasis; a chemotherapeutic side effect, a radiotherapy side effect; aging-related diseases and disorders (e.g., kyphosis, renal dysfunction, frailty, hair loss, hearing loss, muscle fatigue, skin conditions, sarcopenia, and herniated intervertebral disc) and other age-related diseases that are induced by senescence (e.g., diseases/disorders resulting from irradiation, chemotherapy, smoking tobacco, eating a high fat/high sugar diet, and environmental factors); wound healing; skin nevi; fibrotic diseases and disorders (e.g., cystic fibrosis, renal fibrosis, liver fibrosis, pulmonary fibrosis, oral submucous fibrosis, cardiac fibrosis, and pancreatic fibrosis). In certain embodiments, any one or more of the diseases or disorders described above or herein may be excluded.

In some embodiments, methods are provided for treating a senescence-associated disease or disorder by killing senescent cells (i.e., established senescent cells) associated with the disease or disorder in a subject who has the disease or disorder by administering a senolytic agent, where the disease or disorder is osteoarthritis, idiopathic pulmonary fibrosis, chronic obstructive pulmonary disease (COPD), or atherosclerosis.

### Cardiovascular Diseases and Disorders

In other embodiments, the senescence-associated disease or disorder treated by the methods described herein is a cardiovascular disease. The cardiovascular disease may be any one or more of angina, arrhythmia, atherosclerosis, cardiomyopathy, congestive heart failure, coronary artery disease (CAD), carotid artery disease, endocarditis, heart attack (coronary thrombosis, myocardial infarction [MI]), high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease (e.g., peripheral artery disease (PAD)), cardiac stress resistance and stroke.

In certain embodiments, methods are provided for treating senescence-associated cardiovascular disease that is associated with or caused by arteriosclerosis (i.e., hardening of the arteries). The cardiovascular disease may be any one or more of atherosclerosis (e.g., coronary artery disease (CAD) and carotid artery disease); angina, congestive heart failure, and peripheral vascular disease (e.g., peripheral artery disease (PAD)). The methods for treating a cardiovascular disease that is associated with or caused by arteriosclerosis may reduce the likelihood of occurrence of high blood pressure/hypertension, angina, stroke, and heart attack (i.e., coronary thrombosis, myocardial infarction (MI)). In certain embodiments, methods are provided for stabilizing atherosclerotic plaque(s) in a blood vessel (e.g., artery) of a subject, thereby reducing the likelihood of occurrence or delaying the occurrence of a thrombotic event, such as stroke or myocardial infraction. In certain embodiments, these methods comprising administration of a senolytic agent, reduce (i.e., cause decrease of) the lipid content of an atherosclerotic plaque in a blood vessel (e.g., artery) of the subject and/or increase the fibrous cap thickness (i.e., cause an increase, enhance or promote thickening of the fibrous cap).

Atherosclerosis is characterized by patchy intimal plaques (atheromas) that encroach on the lumen of medium-sized and large arteries; the plaques contain lipids, inflammatory cells, smooth muscle cells, and connective tissue. Atherosclerosis can affect large and medium-sized arteries, including the coronary, carotid, and cerebral arteries, the aorta and its branches, and major arteries of the extremities. In some embodiments, methods are provided for inhibiting the formation of atherosclerotic plaques (or reducing, diminishing, causing decrease in formation of atherosclerotic plaques) by administering a senolytic agent. In other embodiments, methods are provided for reducing (decreasing, diminishing) the amount (i.e., level) of plaque. Reduction in the amount of plaque in a blood vessel (e.g., artery) may be determined, for example, by a decrease in surface area of the plaque, or by a decrease in the extent or degree (e.g., percent) of occlusion of a blood vessel (e.g., artery), which can be determined by angiography or other visualizing methods used in the cardiovascular art. Also provided herein are methods for increasing the stability (or improving, promoting, enhancing stability) of atherosclerotic plaques that are present in one or more blood vessels (e.g., one or more arteries) of a subject, which methods comprise administering to the subject any one of the senolytic agents described herein.

Subjects suffering from cardiovascular disease can be identified using standard diagnostic methods known in the art for cardiovascular disease. Generally, diagnosis of atherosclerosis and other cardiovascular disease is based on symptoms (e.g., chest pain or pressure (angina), numbness or weakness in arms or legs, difficulty speaking or slurred speech, drooping muscles in face, leg pain, high blood pressure, kidney failure and/or erectile dysfunction), medical history, and/or physical examination of a patient. Diagnosis may be confirmed by angiography, ultrasonography, or other imaging tests. Subjects at risk of developing cardiovascular disease include those having any one or more of predisposing factors, such as a family history of cardiovascular disease and those having other risk factors (i.e., predisposing factors) such as high blood pressure, dyslipidemia, high cholesterol, diabetes, obesity and cigarette smoking, sedentary lifestyle, and hypertension. In certain embodiments, the cardiovascular disease that is a senescent cell associated disease/disorder is atherosclerosis.

The effectiveness of one or more senolytic agents for treating or preventing (i.e., reducing or decreasing the likelihood of developing or occurrence of) a cardiovascular disease (e.g., atherosclerosis) can readily be determined by a person skilled in the medical and clinical arts. One or any combination of diagnostic methods, including physical examination, assessment and monitoring of clinical symptoms, and performance of analytical tests and methods described herein and practiced in the art (e.g., angiography, electrocardiogramon-stress test), may be used for monitoring the health status of the subject. The effects of the treatment of a senolytic agent or pharmaceutical composition comprising the same can be analyzed using techniques known in the art, such as comparing symptoms of patients suffering from or at risk of cardiovascular disease that have received the treatment with those of patients without such a treatment or with placebo treatment.

### Inflammatory and Autoimmune Diseases and Disorders

In certain embodiments, a senescence-associated disease or disorder is an inflammatory disease or disorder, such as by way of non-limiting example, osteoarthritis, which may be treated or prevented (i.e., likelihood of occurrence is reduced) according to the methods described herein that comprise administration of a senolytic agent. Other inflammatory or autoimmune diseases or disorders that may be treated by administering a senolytic agent such as the inhibitors and antagonists described herein include osteoporosis, psoriasis, oral mucositis, rheumatoid arthritis, inflammatory bowel disease, eczema, kyphosis, herniated intervertebral disc, and the pulmonary diseases, COPD and idiopathic pulmonary fibrosis.

Osteoarthritis degenerative joint disease is characterized by fibrillation of the cartilage at sites of high mechanical stress, bone sclerosis, and thickening of the synovium and the joint capsule. Fibrillation is a local surface disorganization involving splitting of the superficial layers of the cartilage. The early splitting is tangential with the cartilage surface, following the axes of the predominant collagen bundles. Collagen within the cartilage becomes disorganized, and proteoglycans are lost from the cartilage surface. In the absence of protective and lubricating effects of proteoglycans in a joint, collagen fibers become susceptible to degradation, and mechanical destruction ensues. Predisposing risk factors for developing osteoarthritis include increasing age, obesity, previous joint injury, overuse of the joint, weak thigh muscles, and genetics. Symptoms of osteoarthritis include sore or stiff joints, particularly the hips, knees, and lower back, after inactivity or overuse; stiffness after resting that goes away after movement; and pain that is worse after activity or toward the end of the day. Osteoarthritis may also affect the neck, small finger joints, the base of the thumb, ankle, and big toe. Chronic inflammation is thought to be the main age-related factor that contributes to osteoarthritis. In combination with aging, joint overuse and obesity appear to promote osteoarthritis.

By selectively killing senescent cells a senolytic agent prevents (i.e., reduces the likelihood of occurrence), reduces or inhibits loss or erosion of proteoglycan layers in a joint, reduces inflammation in the affected joint, and promotes (i.e., stimulates, enhances, induces) production of collagen (e.g., type 2 collagen). Removal of senescent cells causes a reduction in the amount (i.e., level) of inflammatory cytokines, such as IL-6, produced in a joint and inflammation is reduced. Methods are provided herein for treating osteoarthritis, for selectively killing senescent cells in an osteoarthritic joint of a subject, and/or inducing collagen (such as Type 2 collagen) production in the joint of a subject by administering at least one senolytic agent (which may be combined with at least one pharmaceutically acceptable excipient to form a pharmaceutical composition) to the subject. A senolytic agent also may be used for decreasing (inhibiting, reducing) production of metalloproteinase 13 (MMP-13), which degrades collagen in a joint, and for restoring proteoglycan layer or inhibiting loss and/or degradation of the proteoglycan layer. Treatment with the senolytic agent thereby also prevents (i.e., reduces likelihood of occurrence of), inhibits, or decreases erosion, or slows (i.e., decreases rate) erosion of the bone. As described in detail herein, in certain embodiments, the senolytic agent is administered directly to an osteoarthritic joint (e.g., by intra-articularly, topical, transdermal, intradermal, or subcutaneous delivery). Treatment with a senolytic agent can also restore, improve, or inhibit deterioration of strength of a joint. In addition, the methods comprising administering a senolytic agent can reduce joint pain and are therefore useful for pain management of osteoarthritic joints.

The effectiveness of one or more senolytic agents for treatment or prophylaxis of osteoarthritis in a subject and monitoring of a subject who receives one or more senolytic agents can readily be determined by a person skilled in the medical and clinical arts. One or any combination of diagnostic methods, including physical examination (such as determining tenderness, swelling or redness of the affected joint), assessment and monitoring of clinical symptoms (such as pain, stiffness, mobility), and performance of analytical tests and methods described herein and practiced in the art (e.g., determining the level of inflammatory cytokines or chemokines; X-ray images to determine loss of cartilage as shown by a narrowing of space between the bones in a joint; magnetic resonance imaging (MRI), providing detailed images of bone and soft tissues, including cartilage), may be used for monitoring the health status of the subject. The effects of the treatment of one or more senolytic agents can be analyzed by comparing symptoms of patients suffering from or at risk of an inflammatory disease or disorder, such as osteoarthritis, who have received the treatment with those of patients who have not received such a treatment or who have received a placebo treatment.

In certain embodiments, senolytic agents may be used for treating and/or preventing (i.e., decreasing or reducing the likelihood of occurrence) rheumatoid arthritis (RA). Dysregulation of innate and adaptive immune responses characterize rheumatoid arthritis (RA), which is an autoimmune disease the incidence of which increases with age. Rheumatoid arthritis is a chronic inflammatory disorder that typically affects the small joints in hands and feet. Whereas osteoarthritis results from, at least in part, wear and tear of a joint, rheumatoid arthritis affects the lining of joints, resulting in a painful swelling that can lead to bone erosion and joint deformity. RA can sometimes also affect other organs of the body, such as the skin, eyes, lungs and blood vessels. RA can occur in a subject at any age; however, RA usually begins to develop after age 40. The disorder is much more common in women. In certain embodiments of the methods described herein, RA is excluded.

Chronic inflammation may also contribute to other age-related or aging related diseases and disorders, such as kyphosis and osteoporosis. Kyphosis is a severe curvature in the spinal column, and it is frequently seen with normal and premature aging (see, e.g., Katzman et al., J. Orthop. Sports Phys. Ther. 40 (2010) 352-360). Age-related kyphosis often occurs after osteoporosis weakens spinal bones to the point that they crack and compress. A few types of kyphosis target infants or teens. Severe kyphosis can affect lungs, nerves, and other tissues and organs, causing pain and other problems. Kyphosis has been associated with cellular senescence. Characterizing the capability of a senolytic agent for treating kyphosis may be determined in pre-clinical animal models used in the art. By way of example, TTD mice develop kyphosis (see, e.g., de Boer et al., Science 296 (2002) 1276-1279); other mice that may be used include BubR1^{H/H} mice, which are also known to develop kyphosis (see, e.g., Baker et al., Nature 479 (2011)₂₃₂-236). Kyphosis formation is visually measured over time. The level of senescent cells decreased by treatment with the senolytic agent can be determined by detecting the presence of one or more senescent cell associated markers such as by SA-β-Gal staining.

Osteoporosis is a progressive bone disease that is characterized by a decrease in bone mass and density that may lead to an increased risk of fracture, which may be treated or prevented by administration of the senolytic agents described herein. Bone mineral density (BMD) is reduced, bone microarchitecture deteriorates, and the amount and variety of proteins in bone are altered. Osteoporosis is typically diagnosed and monitored by a bone mineral density test. Post-menopausal women or women who have reduced estrogen are most at risk. While both men and women over 75 are at risk, women are twice as likely to develop osteoporosis than men. The level of senescent cells decreased by treatment with the senolytic agent can be determined by detecting the presence of one or more senescent cell associated markers such as by SA-β-Gal staining.

In still other embodiments, an inflammatory/autoimmune disorder that may be treated or prevented (i.e., likelihood of occurrence is reduced) with the senolytic agents described herein includes irritable bowel syndrome (IBS) and inflammatory bowel diseases, such as ulcerative colitis and Crohn's disease. Inflammatory bowel disease (IBD) involves chronic inflammation of all or part of the digestive tract. In addition to life-threatening complications arising from IBD, the disease can be painful and debilitating. Ulcerative colitis is an inflammatory bowel disease that causes long-lasting inflammation in part of the digestive tract. Symptoms usually develop over time, rather than suddenly. Ulcerative colitis usually affects only the innermost lining of the large intestine (colon) and rectum. Crohn's disease is an inflammatory bowel disease that causes inflammation anywhere along the lining of your digestive tract, and often extends deep into affected tissues. This can lead to abdominal pain, severe diarrhea and malnutrition. The inflammation caused by Crohn's disease can involve different areas of the digestive tract. Diagnosis and monitoring of the diseases are performed according to methods and diagnostic tests routinely practiced in the art, including blood tests, colonoscopy, flexible sigmoidoscopy, barium enema, CT scan, MRI, endoscopy, and small intestine imaging.

Other inflammatory or autoimmune diseases that may be treated or prevented (i.e., likelihood of occurrence is reduced) by using a senolytic agent include eczema, psoriasis, osteoporosis, and pulmonary diseases (e.g., chronic obstructive pulmonary disease (COPD), idiopathic pulmonary fibrosis (IPF), asthma), inflammatory bowel disease, and mucositis (including oral mucositis, which in some instances is induced by radiation). Certain fibrosis or fibrotic conditions of organs such as renal fibrosis, liver fibrosis, pancreatic fibrosis, cardiac fibrosis, skin wound healing, and oral submucous fibrosis may be treated with the senolytic agents described herein.

In certain embodiments, the senescent cell associated disorder is an inflammatory disorder of the skin, such as by way of a non-limiting examples, psoriasis and eczema that may be treated or prevented (i.e., likelihood of occurrence is reduced) according to the methods described herein that comprise administration of a senolytic agent. Psoriasis is characterized by abnormally excessive and rapid growth of the epidermal layer of the skin. A diagnosis of psoriasis is usually based on the appearance of the skin. Skin characteristics typical for psoriasis are scaly red plaques, papules, or patches of skin that may be painful and itchy. In psoriasis, cutaneous and systemic overexpression of various proinflammatory cytokines is observed such as IL-6, a key component of the SASP. Eczema is an inflammation of the skin that is characterized by redness, skin swelling, itching and dryness, crusting, flaking, blistering, cracking, oozing, or bleeding. The effectiveness of senolytic agents for treatment of psoriasis and eczema and monitoring of a subject who receives such a senolytic agent can be readily determined by a person skilled in the medical or clinical arts. One or any combination of diagnostic methods, including physical examination (such as skin appearance), assessment of monitoring of clinical symptoms (such as itching, swelling, and pain), and performance of analytical tests and methods described herein and practiced in the art (i.e., determining the level of pro-inflammatory cytokines).

Other immune disorders or conditions that may be treated or prevented (i.e., likelihood of occurrence is reduced) with senolytic agents described herein include conditions resulting from a host immune response to an organ transplant (e.g., kidney, bone marrow, liver, lung, or heart transplant), such as rejection of the transplanted organ. Senolytic agents described herein may also be used for treating or reducing the likelihood of occurrence of graft-vs-host disease.

### Pulmonary Diseases and Disorders

In some embodiments, methods are provided for treating or preventing (i.e., reducing the likelihood of occurrence of) a senescence-associated disease or disorder that is a pulmonary disease or disorder by killing senescent cells (i.e., established senescent cells) associated with the disease or disorder in a subject who has the disease or disorder by administering senolytic agents described herein. Senescence associated pulmonary diseases and disorders include, for example, idiopathic pulmonary fibrosis (IPF), chronic obstructive pulmonary disease (COPD), asthma, cystic fibrosis, bronchiectasis, and emphysema.

COPD is a lung disease defined by persistently poor airflow resulting from the breakdown of lung tissue (emphysema) and the dysfunction of the small airways (obstructive bronchiolitis). Primary symptoms of COPD include shortness of breath, wheezing, chest tightness, chronic cough, and excess sputum production. Elastase from cigarette smoke-activated neutrophils and macrophages disintegrates the extracellular matrix of alveolar structures, resulting in enlarged air spaces and loss of respiratory capacity (see, e.g., Shapiro et al., Am. J. Respir. Cell Mol. Biol. 32 (2005) 367-372). COPD is most commonly caused by tobacco smoke (including cigarette smoke, cigar smoke, secondhand smoke, pipe smoke), occupational exposure (e.g., exposure to dust, smoke or fumes), and pollution, occurring over decades thereby implicating aging as a risk factor for developing COPD.

The processes involved in causing lung damage include, for example, oxidative stress produced by the high concentrations of free radicals in tobacco smoke; cytokine release due to inflammatory response to irritants in the airway; and impairment of anti-protease enzymes by tobacco smoke and free radicals, allowing proteases to damage the lungs. Genetic susceptibility can also contribute to the disease. In about 1% percent of people with COPD, the disease results from a genetic disorder that causes low level production of alpha-1-antitrypsin in the liver. The enzyme is normally secreted into the bloodstream to help protect the lungs.

Pulmonary fibrosis is a chronic and progressive lung disease characterized by stiffening and scarring of the lung, which may lead to respiratory failure, lung cancer, and heart failure. Fibrosis is associated with repair of epithelium. Fibroblasts are activated, production of extracellular matrix proteins is increased, and transdifferentiation to contractile myofibroblasts contribute to wound contraction. A provisional matrix plugs the injured epithelium and provides a scaffold for epithelial cell migration, involving an epithelial-mesenchymal transition (EMT). Blood loss associated with epithelial injury induces platelet activation, production of growth factors, and an acute inflammatory response. Normally, the epithelial barrier heals and the inflammatory response resolves. However, in fibrotic disease the fibroblast response continues, resulting in unresolved wound healing. Formation of fibroblastic foci is a feature of the disease, reflecting locations of ongoing fibrogenesis. As the name connotes, the etiology of IPF is unknown. The involvement of cellular senescence in IPF is suggested by the observations that the incidence of the disease increases with age and that lung tissue in IPF patients is enriched for SA-β-Gal-positive cells and contains elevated levels of the senescence marker p21 (see, e.g., Minagawa et al., Am. J. Physiol. Lung Cell. Mol. Physiol. 300 (2011) L391-L401; see also, e.g., Naylor et al., supra). Short telomeres are a risk factor common to both IPF and cellular senescence (see, e.g., Alder et al., Proc. Natl. Acad. Sci. USA 105 (2008) 13051-13056). Without wishing to be bound by theory, the contribution of cellular senescence to IPF is suggested by the report that SASP components of senescent cells, such as IL-6, IL-8, and IL-10, promote fibroblast-to-myofibroblast differentiation and epithelial-mesenchymal transition, resulting in extensive remodeling of the extracellular matrix of the alveolar and interstitial spaces (see, e.g., Minagawa et al., supra).

Subjects at risk of developing pulmonary fibrosis include those exposed to environmental or occupational pollutants, such as asbestosis and silicosis; who smoke cigarettes; having some typical connective tissue diseases such as rheumatoid arthritis, SLE and scleroderma; having other diseases that involve connective tissue, such as sarcoidosis and Wegener's granulomatosis; having infections; taking certain medications (e.g., amiodarone, bleomycin, busulfan, methotrexate, and nitrofurantoin); those subject to radiation therapy to the chest; and those whose family member has pulmonary fibrosis.

Symptoms of COPD may include any one of shortness of breath, especially during physical activities; wheezing; chest tightness; having to clear your throat first thing in the morning because of excess mucus in the lungs; a chronic cough that produces sputum that may be clear, white, yellow or greenish; blueness of the lips or fingernail beds (cyanosis); frequent respiratory infections; lack of energy; unintended weight loss (observed in later stages of disease). Subjects with COPD may also experience exacerbations, during which symptoms worsen and persist for days or longer. Symptoms of pulmonary fibrosis are known in the art and include shortness of breath, particularly during exercise; dry, hacking cough; fast, shallow breathing; gradual unintended weight loss; tiredness; aching joints and muscles; and clubbing (widening and rounding of the tips of the fingers or toes).

Subjects suffering from COPD or pulmonary fibrosis can be identified using standard diagnostic methods routinely practiced in the art. Monitoring the effect of one or more senolytic agents administered to a subject who has or who is at risk of developing a pulmonary disease may be performed using the methods typically used for diagnosis. Generally, one or more of the following exams or tests may be performed: physical exam, patient's medical history, patient's family's medical history, chest X-ray, lung function tests (such as spirometry), blood test (e.g., arterial blood gas analysis), bronchoalveolar lavage, lung biopsy, CT scan, and exercise testing.

Other pulmonary diseases or disorders that may be treated by using a senolytic agent include, for example, emphysema, asthma, bronchiectasis, and cystic fibrosis (see, e.g., Fischer et al., Am J Physiol Lung Cell Mol Physiol. 304 (6) (2013) L394-400). These diseases may also be exacerbated by tobacco smoke (including cigarette smoke, cigar smoke, secondhand smoke, pipe smoke), occupational exposure (e.g., exposure to dust, smoke or fumes), infection, and/or pollutants that induce cells into senescence and thereby contribute to inflammation. Emphysema is sometimes considered as a subgroup of COPD.

Bronchiectasis results from damage to the airways that causes them to widen and become flabby and scarred. Bronchiectasis usually is caused by a medical condition that injures the airway walls or inhibits the airways from clearing mucus. Examples of such conditions include cystic fibrosis and primary ciliary dyskinesia (PCD). When only one part of the lung is affected, the disorder may be caused by a blockage rather than a medical condition.

The methods described herein for treating or preventing (i.e., reducing the likelihood or occurrence of) a senescence associated pulmonary disease or disorder may also be used for treating a subject who is aging and has loss (or degeneration) of pulmonary function (i.e., declining or impaired pulmonary function compared with a younger subject) and/or degeneration of pulmonary tissue. The respiratory system undergoes various anatomical, physiological and immunological changes with age. The structural changes include chest wall and thoracic spine deformities that can impair the total respiratory system compliance resulting in increased effort to breathe. The respiratory system undergoes structural, physiological, and immunological changes with age. An increased proportion of neutrophils and lower percentage of macrophages can be found in bronchoalveolar lavage (BAL) of older adults compared with younger adults. Persistent low-grade inflammation in the lower respiratory tract can cause proteolytic and oxidant-mediated injury to the lung matrix resulting in loss of alveolar unit and impaired gas exchange across the alveolar membrane seen with aging. Sustained inflammation of the lower respiratory tract may predispose older adults to increased susceptibility to toxic environmental exposure and accelerated lung function decline. (See, for example, Sharma et al., Clinical Interventions in Aging 1 (2006)₂₅₃-260). Oxidative stress exacerbates inflammation during aging (see, e.g., Brod, Inflamm. Res. 49 (2000) 561-570; Hendel et al., Cell Death and Differentiation 17 (2010) 596-606). Alterations in redox balance and increased oxidative stress during aging precipitate the expression of cytokines, chemokines, and adhesion molecules, and enzymes (see, e.g., Chung et al., Ageing Res. Rev. 8 (2009) 18-30). Constitutive activation and recruitment of macrophages, T cells, and mast cells foster release of proteases leading to extracellular matrix degradation, cell death, remodeling, and other events that can cause tissue and organ damage during chronic inflammation (see, e.g., Demedts et al., Respir. Res. 7 (2006) 53-63). By administering a senolytic agent to an aging subject (which includes a middle-aged adult who is asymptomatic), the decline in pulmonary function may be decelerated or inhibited by killing and removing senescent cells from the respiratory tract.

The effectiveness of a senolytic agent can readily be determined by a person skilled in the medical and clinical arts. One or any combination of diagnostic methods, including physical examination, assessment and monitoring of clinical symptoms, and performance of analytical tests and methods described herein, may be used for monitoring the health status of the subject. The effects of the treatment of a senolytic agent or pharmaceutical composition comprising the agent can be analyzed using techniques known in the art, such as comparing symptoms of patients suffering from or at risk of the pulmonary disease that have received the treatment with those of patients without such a treatment or with placebo treatment. In addition, methods and techniques that evaluate mechanical functioning of the lung, for example, techniques that measure lung capacitance, elastance, and airway hypersensitivity may be performed. To determine lung function and to monitor lung function throughout treatment, any one of numerous measurements may be obtained, expiratory reserve volume (ERV), forced vital capacity (FVC), forced expiratory volume (FEV) (e.g., FEV in one second, FEV1), FEV1/FEV ratio, forced expiratory flow 25% to 75%, and maximum voluntary ventilation (MVV), peak expiratory flow (PEF), slow vital capacity (SVC). Total lung volumes include total lung capacity (TLC), vital capacity (VC), residual volume (RV), and functional residual capacity (FRC). Gas exchange across alveolar capillary membrane can be measured using diffusion capacity for carbon monoxide (DLCO). Peripheral capillary oxygen saturation (SpO₂) can also be measured; normal oxygen levels are typically between 95% and 100%. An SpO₂ level below 90% suggests the subject has hypoxemia. Values below 80% are considered critical and requiring intervention to maintain brain and cardiac function and avoid cardiac or respiratory arrest.

### Neurological Diseases and Disorders

Senescence-associated diseases or disorders treatable by administering a senolytic agent described herein include neurological diseases or disorders. Such senescence-associated diseases and disorders include Parkinson's disease, Alzheimer's disease (and other dementias), motor neuron dysfunction (MND), mild cognitive impairment (MCI), Huntington's disease and diseases and disorders of the eyes, such as age-related macular degeneration. Other diseases of the eye that are associated with increasing age are glaucoma, vision loss, presbyopia, and cataracts.

Parkinson's disease (PD) is the second most common neurodegenerative disease. It is a disabling condition of the brain characterized by slowness of movement (bradykinesia), shaking, stiffness and in the later stages, loss of balance. Many of these symptoms are due to the loss of certain nerves in the brain, which results in the lack of dopamine. This disease is characterized by neurodegeneration, such as the loss of about 50% to 70% of the dopaminergic neurons in the substantia nigra pars compacta, a profound loss of dopamine in the striatum and/or the presence of intracytoplasmic inclusions (Lewy bodies), which are composed mainly of alpha-synuclein and ubiquitin. Parkinson's disease also features locomotor deficits, such as tremor, rigidity, bradykinesia and/or postural instability. Subjects at risk of developing Parkinson's disease include those having a family history of Parkinson's disease and those exposed to pesticides (e.g., rotenone or paraquat), herbicides (e.g., agent orange), or heavy metals. Senescence of dopamine-producing neurons is thought to contribute to the observed cell death in PD through the production of reactive oxygen species (see, e.g., Cohen et al., J. Neural Transm. Suppl. 19 (1983) 89-103); therefore, the methods and senolytic agents described herein are useful for treatment and prophylaxis of Parkinson's disease.

Methods for detecting, monitoring or quantifying neurodegenerative deficiencies and/or locomotor deficits associated with Parkinson's diseases are known in the art, such as histological studies, biochemical studies, and behavioral assessment (see, e.g., U.S. Application Publication No. 2012/0005765). Symptoms of Parkinson's disease are known in the art and include, but are not limited to, difficulty starting or finishing voluntary movements, jerky, stiff movements, muscle atrophy, shaking (tremors), and changes in heart rate, but normal reflexes, bradykinesia, and postural instability. There is a growing recognition that people diagnosed with Parkinson's disease may have cognitive impairment, including mild cognitive impairment, in addition to their physical symptoms.

Alzheimer's disease (AD) is a neurodegenerative disease that shows a slowly progressive mental deterioration with failure of memory, disorientation, and confusion, leading to profound dementia. Age is the single greatest predisposing risk factor for developing AD, which is the leading cause of dementia in the elderly (see, e.g., Hebert, et al., Arch. Neural. 60 (2003) 1119-1122). Early clinical symptoms show remarkable similarity to mild cognitive impairment (see below). As the disease progresses, impaired judgment, confusion, behavioral changes, disorientation, and difficulty in walking and swallowing occur.

Alzheimer's disease is characterized by the presence of neurofibrillary tangles and amyloid (senile) plaques in histological specimens. The disease predominantly involves the limbic and cortical regions of the brain. The argyrophilic plaques containing the amyloidogenic AD fragment of amyloid precursor protein (APP) are scattered throughout the cerebral cortex and hippocampus. Neurofibrillary tangles are found in pyramidal neurons predominantly located in the neocortex, hippocampus, and nucleus basalis of Meynert. Other changes, such as granulovacuolar degeneration in the pyramidal cells of the hippocampus and neuron loss and gliosis in the cortex and hippocampus, are observed. Subjects at risk of developing Alzheimer's disease include those of advanced age, those with a family history of Alzheimer's disease, those with genetic risk genes (e.g., ApoE4) or deterministic gene mutations (e.g., APP, PS1, or PS2), and those with history of head trauma or heart/vascular conditions (e.g., high blood pressure, heart disease, stroke, diabetes, high cholesterol, etc.).

A number of behavioral and histopathological assays are known in the art for evaluating Alzheimer's disease phenotype, for characterizing therapeutic agents, and assessing treatment. Histological analyses are typically performed postmortem. Histological analysis of AB levels may be performed using Thioflavin-S, Congo red, or anti-A staining (e.g., 4G8, 10D5, or 6E10 antibodies) to visualize A deposition on sectioned brain tissues (see, e.g., Holcomb et al., Nat. Med. 4 (1998) 97-100; Borchelt et al., Neuron 19 (1997) 939-945; Dickson et al., Am. J. Path. 132 (1998) 86-101). In vivo methods of visualizing Aß deposition in transgenic mice have been also described. BSB ((trans, trans)-1-bromo-2,5-bis-(3-hydroxycarbonyl-4-hydroxy) styrylbenzene) and PET tracer ¹¹C-labelled Pittsburgh Compound-B (PIB) bind to AP plaques (see, e.g., Skovronsky et al., Proc. Natl. Acad. Sci. USA 97 (2000) 7609-7614; Klunk et al., Ann. Neurol. 55 (2004) 306-319). ¹⁹F-containing amyloidophilic Congo red-type compound FSB ((E,E)-1-fluoro-2,5-bis-(3-hydroxycarbonyl-4-hydroxy) styrylbenzene) allows visualization of Aß plaques by MRI (see, e.g., Higuchi et al., Nature Neurosci. 8 (2005) 527-533). Radiolabeled, putrescine-modified amyloid-beta peptide labels amyloid deposits in vivo in a mouse model of Alzheimer's disease (see, e.g., Wengenack et al., Nat. Biotechnol. 18 (2000) 868-872).

Increased glial fibrillary acidic protein (GFAP) by astrocytes is a marker for astroglial activation and gliosis during neurodegeneration. AP plaques are associated with GFAP-positive activated astrocytes, and may be visualized via GFAP staining (see, e.g., Nagele et al., Neurobiol. Aging 25 (2004) 663-674; Mandybur et al., Neurology 40 (1990) 635-639; Liang et al., J. Biol. Chem. 285 (2010)₂₇₇₃₇-27744). Neurofibrillary tangles may be identified by immunohistochemistry using thioflavin-S fluorescent microscopy and Gallyas silver stains (see, e.g., Gotz et al., J. Biol. Chem. 276 (2001) 529-534; U.S. Pat. No. 6,664,443). Axon staining with electron microscopy and axonal transport studies may be used to visualize neuronal degeneration (see, e.g., Ishihara et al., Neuron 24 (1999) 751-762).

Subjects suffering from Alzheimer's disease can be identified using standard diagnostic methods known in the art for Alzheimer's disease. Generally, diagnosis of Alzheimer's disease is based on symptoms (e.g., progressive decline in memory function, gradual retreat from and frustration with normal activities, apathy, agitation or irritability, aggression, anxiety, sleep disturbance, dysphoria, aberrant motor behavior, disinhibition, social withdrawal, decreased appetite, hallucinations, dementia), medical history, neuropsychological tests, neurological and/or physical examination of a patient. Cerebrospinal fluid may also be evaluated for various proteins that have been associated with Alzheimer pathology, including tau, amyloid beta peptide, and AD7C-NTP. Genetic testing is also available for early-onset familial Alzheimer disease (eFAD), an autosomal-dominant genetic disease. Clinical genetic testing is available for individuals with AD symptoms or at-risk family members of patients with early-onset disease. In the U.S., mutations for PS2, and APP may be evaluated in a clinical or federally approved laboratory under the Clinical Laboratory Improvement Amendments. A commercial test for PS1 mutations is also available (Elan Pharmaceuticals).

The effectiveness of one or more senolytic agents described herein and monitoring of a subject who receives one or more senolytic agents can readily be determined by a person skilled in the medical and clinical arts. One or any combination of diagnostic methods, including physical examination, assessment and monitoring of clinical symptoms, and performance of analytical tests and methods described herein, may be used for monitoring the health status of the subject. The effects of administering one or more senolytic agents can be analyzed using techniques known in the art, such as comparing symptoms of patients suffering from or at risk of Alzheimer's disease that have received the treatment with those of patients without such a treatment or with placebo treatment.

Mild Cognitive Impairment (MCI) is a brain-function syndrome involving the onset and evolution of cognitive impairments beyond those expected based on age and education of the individual, but which are not significant enough to interfere with the daily activities of an individual. MCI is an aspect of cognitive aging that is considered to be a transitional state between normal aging and dementia into which it may convert (see, Pepeu, Dialogues in Clinical Neuroscience 6 (2004) 369-377). MCI that primarily affects memory is known as “amnestic MCI.” A person with amnestic MCI may start to forget important information that he or she would previously have recalled easily, such as recent events. Amnestic MCI is frequently seen as prodromal stage of Alzheimer's disease. MCI that affects thinking skills other than memory is known as “non-amnestic MCI.” This type of MCI affect thinking skills such as the ability to make sound decisions, judge the time or sequence of steps needed to complete a complex task, or visual perception. Individuals with non-amnestic MCI are believed to be more likely to convert to other types of dementia (e.g., dementia with Lewy bodies).

Persons in the medical art have a growing recognition that people diagnosed with Parkinson's disease may have MCI in addition to their physical symptoms. Recent studies show 20-30% of people with Parkinson's disease have MCI and that their MCI tends to be non-amnestic. Parkinson's disease patients with MCI sometimes go on to develop full blown dementia (Parkinson's disease with dementia).

Methods for detecting, monitoring, quantifying or assessing neuropathological deficiencies associated with MCI are known in the art, including astrocyte morphological analyses, release of acetylcholine, silver staining for assessing neurodegeneration, and PiB PET imaging to detect beta amyloid deposits (see, e.g., U.S. Application Publication No. 2012/0071468; Pepeu, (2004), supra). Methods for detecting, monitoring, quantifying or assessing behavioral deficiencies associated with MCI are also known in the art, including eight-arm radial maze paradigm, non-matching-to-sample task, allocentric place determination task in a water maze, Morri's maze test, visuospatial tasks, delayed response spatial memory task, and the olfactory novelty test.

Motor Neuron Dysfunction (MND) is a group of progressive neurological disorders that destroy motor neurons, the cells that control essential voluntary muscle activity such as speaking, walking, breathing and swallowing. It is classified according to whether degeneration affects upper motor neurons, lower motor neurons, or both. Examples of MNDs include but are not limited to Amyotrophic Lateral Sclerosis (ALS), also known as Lou Gehrig's Disease, progressive bulbar palsy, pseudobulbar palsy, primary lateral sclerosis, progressive muscular atrophy, lower motor neuron disease, and spinal muscular atrophy (SMA) (e.g., SMA1 also called Werdnig-Hoffmann Disease, SMA2, SMA3 also called Kugelberg-Welander Disease, and Kennedy's disease), post-polio syndrome, and hereditary spastic paraplegia. In adults, the most common MND is amyotrophic lateral sclerosis (ALS), which affects both upper and lower motor neurons. It can affect the arms, legs, or facial muscles. Primary lateral sclerosis is a disease of the upper motor neurons, while progressive muscular atrophy affects only lower motor neurons in the spinal cord. In progressive bulbar palsy, the lowest motor neurons of the brain stem are most affected, causing slurred speech and difficulty chewing and swallowing. There are almost always mildly abnormal signs in the arms and legs. Patients with MND exhibit a phenotype of Parkinson's disease (e.g., having tremor, rigidity, bradykinesia, and/or postural instability). Methods for detecting, monitoring or quantifying locomotor and/or other deficits associated with Parkinson's diseases, such as MND, are known in the art (see, e.g., U.S. Application Publication No. 2012/0005765).

Methods for detecting, monitoring, quantifying or assessing motor deficits and histopathological deficiencies associated with MND are known in the art, including histopathological, biochemical, and electrophysiological studies and motor activity analysis (see, e.g., Rich et al., J. Neurophysiol. 88 (2002) 3293-3304; Appel et al., Proc. Natl. Acad. Sci. USA 88 (1991) 647-651). Histopathologically, MNDs are characterized by death of motor neurons, progressive accumulation of detergent-resistant aggregates containing SOD1 and ubiquitin and aberrant neurofilament accumulations in degenerating motor neurons. In addition, reactive astroglia and microglia are often detected in diseased tissue. Patients with an MND show one or more motor deficits, including muscle weakness and wasting, uncontrollable twitching, spasticity, slow and effortful movements, and overactive tendon reflexes.

### Ophthalmic Diseases and Disorders

In certain embodiments, a senescence-associated disease or disorder is an ocular disease, disorder, or condition, for example, presbyopia, macular degeneration, or cataracts. In other certain embodiments, the senescence-associated disease or disorder is glaucoma. Macular degeneration is a neurodegenerative disease that causes the loss of photoreceptor cells in the central part of retina, called the macula. Macular degeneration generally is classified into two types: dry type and wet type. The dry form is more common than the wet, with about 90% of age-related macular degeneration (ARMD or AMD) patients diagnosed with the dry form. The wet form of the disease usually leads to more serious vision loss. While the exact causes of age-related macular degeneration are still unknown, the number of senescent retinal pigmented epithelial (RPE) cells increases with age. Age and certain genetic factors and environmental factors are risk factors for developing ARMD (see, e.g., Lyengar et al., Am. J. Hum. Genet. 74 (2004)₂₀-39; Kenealy et al., Mol. Vis. 10 (2004) 57-61; Gorin et al., Mol. Vis. 5 (1999)₂₉). Environment predisposing factors include omega-3 fatty acids intake (see, e.g., Christen et al., Arch. Ophthalmol. 129 (2011) 921-929); estrogen exposure (see, e.g., Feshanich et al., Arch. Ophthalmol. 126 (4) (2008) 519-524); and increased serum levels of vitamin D (see, e.g., Millen, et al., Arch. Ophthalmol. 129 (4) (2011) 481-89). Genetic predisposing risk factors include reduced levels Dicer1 (enzyme involved in maturation of micro-RNA) in eyes of patients with dry AMD and decreased micro RNAs contributes to a senescent cell profile.

Dry ARMD is associated with atrophy of RPE layer, which causes loss of photoreceptor cells. The dry form of ARMD may result from aging and thinning of macular tissues and from deposition of pigment in the macula. Senescence appears to inhibit both replication and migration of RPE, resulting in permanent RPE depletion in the macula of dry AMD patients (see, e.g., Iriyama et al., J. Biol. Chem. 283 (2008) 11947-11953). With wet ARMD, new blood vessels grow beneath the retina and leak blood and fluid. This abnormal leaky choroidal neovascularization causes the retinal cells to die, creating blind spots in central vision. Different forms of macular degeneration may also occur in younger patients. Non-age-related etiology may be linked to heredity, diabetes, nutritional deficits, head injury, infection, or other factors.

Declining vision noticed by the patient or by an ophthalmologist during a routine eye exam may be the first indicator of macular degeneration. The formation of exudates, or “drusen,” underneath the Bruch's membrane of the macula is often the first physical sign that macular degeneration may develop. Symptoms include perceived distortion of straight lines and, in some cases, the center of vision appears more distorted than the rest of a scene; a dark, blurry area or “white-out” appears in the center of vision; and/or color perception changes or diminishes. Diagnosing and monitoring of a subject with macular degeneration may be accomplished by a person skilled in the ophthalmic art according to art-accepted periodic eye examination procedures and report of symptoms by the subject.

Presbyopia is an age-related condition where the eye exhibits a progressively diminished ability to focus on near objects as the speed and amplitude of accommodation of a normal eye decrease with advancing age. Loss of elasticity of the crystalline lens and loss of contractility of the ciliary muscles have been postulated as its cause (see, e.g., Heys et al., Mol. Vis. 10 (2004) 956-963; Petrash, Invest. Ophthalmol. Vis. Sci. 54 (2013) ORSF54-ORSF59). Age-related changes in the mechanical properties of the anterior lens capsule and posterior lens capsule suggest that the mechanical strength of the posterior lens capsule decreases significantly with age (see, e.g., Krag et al., Invest. Ophthalmol. Vis. Sci. 44 (2003) 691-696; Krag et al., Invest. Ophthalmol. Vis. Sci. 38 (1997) 357-363).

The laminated structure of the capsule also changes and may result, at least in part, from a change in the composition of the tissue (see, e.g., Krag et al., 1997, supra, and references cited therein). The major structural component of the lens capsule is basement membrane type IV collagen that is organized into a three-dimensional molecular network (see, e.g., Cummings et al., Connect. Tissue Res. 55 (2014) 8-12; Veis et al., Coll. Relat. Res. 1 (1981)₂₆₉-286). Type IV collagen is composed of six homologous a chains (a 1-6) that associate into heterotrimeric collagen IV protomers with each comprising a specific chain combination of a 112, a 345, or a 556 (see, e.g., Khoshnoodi et al., Microsc. Res. Tech. 71 (2008) 357-370). Protomers share structural similarities of a triple-helical collagenous domain with the triplet peptide sequence of Gly-X-Y (Timpl et al., Eur. J. Biochem. 95 (1979)₂₅₅-263), ending in a globular C-terminal region termed the non-collagenous 1 (NC1) domain. The N-termini are composed of a helical domain termed the 7S domain (see, e.g., Risteli et al., Eur. J. Biochem. 108 (1980)₂₃₉-250), which is also involved in protomer-protomer interactions.

Research has suggested that collagen IV influences cellular function which is inferred from the positioning of basement membranes underneath epithelial layers, and data support the role of collagen IV in tissue stabilization (see, e.g., Cummings et al., supra). Posterior capsule opacification (PCO) develops as a complication in approximately 20-40% of patients in subsequent years after cataract surgery (see, e.g., Awasthi et al., Arch. Ophthalmol. 127 (2009) 555-562). PCO results from proliferation and activity of residual lens epithelial cells along the posterior capsule in a response akin to wound healing. Growth factors, such as fibroblast growth factor, transforming growth factor, epidermal growth factor, hepatocyte growth factor, insulin-like growth factor, and interleukins IL-1 and IL-6 may also promote epithelial cell migration, (see, e.g., Awasthi et al, supra; Raj et al., supra). As discussed herein, production of these factors and cytokines by senescent cells contribute to the SASP. In contrast, in vitro studies show that collagen IV promotes adherence of lens epithelial cells (see, e.g., Olivero et al., Invest. Ophthalmol. Vis. Sci. 34 (1993)₂₈₂₅-2834). Adhesion of collagen IV, fibronectin, and laminin to the intraocular lens inhibits cell migration and may reduce the risk of PCO (see, e.g., Raj et al, Int. J. Biomed. Sci. 3 (2007)₂₃₇-250).

Without wishing to be bound by any particular theory, selective killing of senescent cells by the senolytic agents described herein may slow or impede (delay, inhibit, retard) the disorganization of the type IV collagen network. Removal of senescent cells and thereby removing the inflammatory effects of SASP may decrease or inhibit epithelial cell migration and may also delay (suppress) the onset of presbyopia or decrease or slow the progressive severity of the condition (such as slow the advancement from mild to moderate or moderate to severe). The senolytic agents described herein may also be useful for post-cataract surgery to reduce the likelihood of occurrence of PCO.

While no direct evidence for the involvement of cellular senescence with the development of cataracts has been obtained from human studies, BubR1 hypomorphic mice develop posterior subcapsular cataracts bilaterally early in life, suggesting that senescence may play a role (see, e.g., Baker et al., Nat. Cell Biol. 10 (2008) 825-836). Cataracts are a clouding of the lens of an eye, causing blurred vision, and if left untreated can result in blindness. Surgery is effective and routinely performed to remove cataracts. Administration of one or more of the senolytic agents described herein may result in decreasing the likelihood of occurrence of a cataract or may slow or inhibit progression of a cataract. The presence and severity of a cataract can be monitored by eye exams using methods routinely performed by a person skilled in the ophthalmology art.

In certain embodiments, at least one senolytic agent described herein may be administered to a subject who is at risk of developing presbyopia, cataracts, or macular degeneration. Treatment with a senolytic agent may be initiated when a human subject is at least 40 years of age to delay or inhibit onset or development of cataracts, presbyopia, and macular degeneration. Because almost all humans develop presbyopia, in certain embodiments, the senolytic agent may be administered in a manner as described herein to a human subject after the subject reaches the age of 40 to delay or inhibit onset or development of presbyopia.

In certain embodiments, the senescence associated disease or disorder is glaucoma. Glaucoma is a broad term used to describe a group of diseases that causes visual field loss, often without any other prevailing symptoms. The lack of symptoms often leads to a delayed diagnosis of glaucoma until the terminal stages of the disease. Even if subjects afflicted with glaucoma do not become blind, their vision is often severely impaired. Normally, clear fluid flows into and out of the front part of the eye, known as the anterior chamber. In individuals who have open/wide-angle glaucoma, this fluid drains too slowly, leading to increased pressure within the eye. If left untreated, this high pressure subsequently damages the optic nerve and can lead to complete blindness. The loss of peripheral vision is caused by the death of ganglion cells in the retina. Ganglion cells are a specific type of projection neuron that connects the eye to the brain. When the cellular network required for the outflow of fluid was subjected to SA-β-Gal staining, a fourfold increase in senescence has been observed in glaucoma patients (see, e.g., Liton et al., Exp. Gerontol. 40 (2005) 745-748).

For monitoring the effect of a therapy on inhibiting progression of glaucoma, standard automated perimetry (visual field test) is the most widely used technique. In addition, several algorithms for progression detection have been developed (see, e.g., Wesselink et al., Arch. Ophthalmol. 127 (3) (2009)₂₇₀-274, and references therein). Additional methods include gonioscopy (examines the trabecular meshwork and the angle where fluid drains out of the eye); imaging technology, for example scanning laser tomography (e.g., HRT3), laser polarimetry (e.g., GDX), and ocular coherence tomography); ophthalmoscopy; and pachymeter measurements that determine central corneal thickness.

### Metabolic Diseases or Disorders

Senescence-associated diseases or disorders treatable by administering a senolytic agent include metabolic diseases or disorders. Such senescent cell associated diseases and disorders include diabetes, metabolic syndrome, diabetic ulcers, and obesity.

Diabetes is characterized by high levels of blood glucose caused by defects in insulin production, insulin action, or both. The great majority (90 to 95%) of all diagnosed cases of diabetes in adults are type 2 diabetes, characterized by the gradual loss of insulin production by the pancreas. Diabetes is the leading cause of kidney failure, nontraumatic lower-limb amputations, and new cases of blindness among adults in the U.S. Diabetes is a major cause of heart disease and stroke and is the seventh leading cause of death in the U.S. (see, e.g., Centers for Disease Control and Prevention, National diabetes fact sheet: national estimates and general information on diabetes and pre-diabetes in the United States, 2011 (“Diabetes fact sheet”)). Senolytic agents described herein may be used for treating type 2 diabetes, particularly age-, diet- and obesity-associated type 2 diabetes.

Involvement of senescent cells in metabolic disease, such as obesity and type 2 diabetes, has been suggested as a response to injury or metabolic dysfunction (see, e.g., Tchkonia et al., Aging Cell 9 (2010) 667-684). Fat tissue from obese mice showed induction of the senescence markers SA-β-Gal, p53, and p21 (see, e.g., Tchkonia et al., supra; Minamino et al., Nat. Med. 15 (2009) 1082-1087). A concomitant up-regulation of pro-inflammatory cytokines, such as tumor necrosis factor and Ccl2/MCP1, was observed in the same fat tissue (see, e.g., Minamino et al., supra). Induction of senescent cells in obesity potentially has clinical implications because pro-inflammatory SASP components are also suggested to contribute to type 2 diabetes (see, e.g., Tchkonia et al., supra). A similar pattern of up-regulation of senescence markers and SASP components are associated with diabetes, both in mice and in humans (see, e.g., Minamino et al., supra). Accordingly, the methods described herein that comprise administering a senolytic agent may be useful for treatment or prophylaxis of type 2 diabetes, as well as obesity and metabolic syndrome. Without wishing to be bound by theory, contact of senescent pre-adipocytes with a senolytic agent thereby killing the senescent pre-adipocytes may provide clinical and health benefit to a person who has any one of diabetes, obesity, or metabolic syndrome.

Subjects suffering from type 2 diabetes can be identified using standard diagnostic methods known in the art for type 2 diabetes. Generally, diagnosis of type 2 diabetes is based on symptoms (e.g., increased thirst and frequent urination, increased hunger, weight loss, fatigue, blurred vision, slow-healing sores or frequent infections, and/or areas of darkened skin), medical history, and/or physical examination of a patient. Subjects at risk of developing type 2 diabetes include those who have a family history of type 2 diabetes and those who have other risk factors such as excess weight, fat distribution, inactivity, race, age, prediabetes, and/or gestational diabetes.

The effectiveness of a senolytic agent can readily be determined by a person skilled in the medical and clinical arts. One or any combination of diagnostic methods, including physical examination, assessment and monitoring of clinical symptoms, and performance of analytical tests and methods, such as those described herein, may be used for monitoring the health status of the subject. A subject who is receiving one or more senolytic agents described herein for treatment or prophylaxis of diabetes can be monitored, for example, by assaying glucose and insulin tolerance, energy expenditure, body composition, fat tissue, skeletal muscle, and liver inflammation, and/or lipotoxicity (muscle and liver lipid by imaging in vivo and muscle, liver, bone marrow, and pancreatic-cell lipid accumulation and inflammation by histology). Other characteristic features or phenotypes of type 2 diabetes are known and can be assayed as described herein and by using other methods and techniques known and routinely practiced in the art.

Obesity and obesity-related disorders are used to refer to conditions of subjects who have a body mass that is measurably greater than ideal for their height and frame. Body Mass Index (BMI) is a measurement tool used to determine excess body weight and is calculated from the height and weight of a subject. A human is considered overweight when the person has a BMI of 25-29; a person is considered obese when the person has a BMI of 30-39, and a person is considered severely obese when the person has a BMI of >40. Accordingly, the terms obesity and obesity-related refer to human subjects with body mass index values of greater than 30, greater than 35, or greater than 40. A category of obesity not captured by BMI is called “abdominal obesity” in the art, which relates to the extra fat found around a subject's middle, which is an important factor in health, even independent of BMI. The simplest and most often used measure of abdominal obesity is waist size. Generally abdominal obesity in women is defined as a waist size 35 inches or higher, and in men as a waist size of 40 inches or higher. More complex methods for determining obesity require specialized equipment, such as magnetic resonance imaging or dual energy X-ray absorptiometry machines.

A condition or disorder associated with diabetes and senescence is a diabetic ulcer (i.e., diabetic wound). An ulcer is a breakdown in the skin, which may extend to involve the subcutaneous tissue or even muscle or bone. These lesions occur, particularly on the lower extremities. Patients with diabetic venous ulcer exhibit elevated presence of cellular senescence at sites of chronic wounds (see, e.g., Stanley et al., J. Vas. Surg. 33 (2001) 1206-1211). Chronic inflammation is also observed at sites of chronic wounds, such as diabetic ulcers (see, e.g., Goren et al., Am. J. Pathol. 168 (2006) 65-77) suggesting that the proinflammatory cytokine phenotype of senescent cells has a role in the pathology.

Subjects who have type 2 diabetes or who are at risk of developing type 2 diabetes may have metabolic syndrome. Metabolic syndrome in humans is typically associated with obesity and characterized by one or more cardiovascular disease, liver steatosis, hyperlipidemia, diabetes, and insulin resistance. A subject with metabolic syndrome may present with a cluster of metabolic disorders or abnormalities which may include, for example, one or more of hypertension, type-2 diabetes, hyperlipidemia, dyslipidemia (e.g., hypertriglyceridemia, hypercholesterolemia), insulin resistance, liver steatosis (steatohepatitis), hypertension, atherosclerosis, and other metabolic disorders.

### Renal Dysfunction

Nephrological pathologies, such as glomerular disease, arise in the elderly and may be treated by the administration of senolytic compounds described herein. Glomerulonephritis is characterized by inflammation of the kidney and by the expression of two proteins, IL1α and IL1ß (see, e.g., Niemir et al., Kidney Int. 52 (1997) 393-403). IL1α and ILIß are considered master regulators of SASP (see, e.g., Coppe et al., PLOS. Biol. 6 (2008)₂₈₅₃-2868). Glomerular disease is associated with elevated presence of senescent cells, especially in fibrotic kidneys (see, e.g., Sis et al., Kidney Int. 71 (2007)₂₁₈-226).

### Dermatological Diseases or Disorders

Diseases or disorders treatable by administering a compound described herein include dermatological diseases or disorders. Such diseases and disorders include psoriasis and eczema, which are also inflammatory diseases and are discussed in greater detail above. Other dermatological diseases and disorders include rhytides (wrinkles due to aging); pruritis (linked to diabetes and aging); dysesthesia (chemotherapy side effect that is linked to diabetes and multiple sclerosis); psoriasis (as noted) and other papulosquamous disorders, for example, erythroderma, lichen planus, and lichenoid dermatosis; atopic dermatitis (a form of eczema and associated with inflammation); eczematous eruptions (often observed in aging patients and linked to side effects of certain drugs). Other dermatological diseases and disorders associated with senescence include eosinophilic dermatosis (linked to certain kinds of hematologic cancers); reactive neutrophilic dermatosis (associated with underlying diseases such as inflammatory bowel syndrome); pemphigus (an autoimmune disease in which autoantibodies form against desmoglein); pemphigoid and other immunobullous dermatosis (autoimmune blistering of skin); fibrohistiocytic proliferations of skin, which is linked to aging; and cutaneous lymphomas that are more common in older populations. Another dermatological disease that may be treatable according to the methods described herein includes cutaneous lupus, which is a symptom of lupus erythematosus. Late onset lupus may be linked to decreased (i.e., reduced) function of T-cell and B-cells and cytokines (immunosenescence). Still other dermatology indications which may be treated using the compounds described herein include, but are not limited to plaque psoriasis, dermatitis and alopecia induced by cancer therapy (chemotherapy or radiation), vitiligo, alopecia areata, hidradenitis suppurativa, chronic spontaneous urticaria, actinic keratosis and seborrheic keratosis.

### Metastasis

In some embodiments, methods are provided for treating or preventing (i.e., reducing the likelihood of occurrence or development of) a senescent cell associated disease (or disorder or condition), which is metastasis. The senolytic agents described herein may also be used according to the methods described herein for treating or preventing (i.e., reducing the likelihood of occurrence of) metastasis (i.e., the spreading and dissemination of cancer or tumor cells) from one organ or tissue to another organ or tissue in the body.

A senescent cell-associated disease or disorder includes metastasis, and a subject who has cancer may benefit from administration of a senolytic agent as described herein for inhibiting metastasis. Such a senolytic agent when administered to a subject who has cancer according to the methods described herein may inhibit tumor proliferation. Metastasis of a cancer occurs when the cancer cells (i.e., tumor cells) spread beyond the anatomical site of origin and initial colonization to other areas throughout the body of the subject. Tumor proliferation may be determined by tumor size, which can be measured in various ways familiar to a person skilled in the art, such as by PET scanning, MRI, CAT scan, biopsy, for example. The effect of the therapeutic agent on tumor proliferation may also be evaluated by examining differentiation of the tumor cells.

As used herein and in the art, the terms cancer or tumor are clinically descriptive terms that encompass diseases typically characterized by cells exhibiting abnormal cellular proliferation. The term cancer is generally used to describe a malignant tumor or the disease state arising from the tumor. Alternatively, abnormal growth may be referred to in the art as a neoplasm. The term tumor, such as in reference to a tissue, generally refers to any abnormal tissue growth that is characterized, at least in part, by excessive and abnormal cellular proliferation. A tumor may be metastatic and capable of spreading beyond its anatomical site of origin and initial colonization to other areas throughout the body of the subject. A cancer may comprise a solid tumor or may comprise a “liquid” tumor (e.g., leukemia and other blood cancers).

Cells are induced to senesce by cancer therapies, such as radiation and certain chemotherapy drugs. The presence of senescent cells increases secretion of inflammatory molecules, promotes tumor progression, which may include promoting tumor growth and increasing tumor size, promoting metastasis, and altering differentiation. When senescent cells are destroyed, tumor progression is significantly inhibited, resulting in tumors of small size and with little or no observed metastatic growth (see, e.g., International Publication No. WO 2013/090645).

In some embodiments, methods are provided for preventing (i.e., reducing the likelihood of occurrence of), inhibiting, or retarding metastasis in a subject who has cancer by administering a senolytic agent as described herein. In other embodiments, the senolytic agent is administered on one or more days within a treatment window (i.e., treatment course) of no longer than 7 days or 14 days. In still other embodiments, the treatment course is no longer than 2, 3, 4, 5, 6, 7, 8, 9, 10, 11, 12, 13, 14, 15, 16, 17, 18, 19, 20, or no longer than 21 days. In still other embodiments, the treatment course is a single day. In still other embodiments, the senolytic agent is administered on two or more days within a treatment window of no longer than 7 days or 14 days.

Because cells may be induced to senesce by cancer therapies, such as radiation and certain chemotherapy drugs (e.g., doxorubicin; paclitaxel; gemcitabine; pomalidomide; lenalidomide), a senolytic agent described herein may be administered after the chemotherapy or radiotherapy to kill (or facilitate killing) of these senescent cells. As discussed herein and understood in the art, establishment of senescence, such as shown by the presence of a senescence-associated secretory phenotype (SASP), occurs over several days; therefore, administering a senolytic agent to kill senescent cells, and thereby reduce the likelihood of occurrence or reduce the extent of metastasis, is initiated when senescence has been established. As discussed herein, the following treatment courses for administration of the senolytic agent may be used in methods described herein for treating or preventing (i.e., reducing the likelihood of occurrence, or reducing the severity) a chemotherapy or radiotherapy side effect.

In certain embodiments, when chemotherapy or radiotherapy is administered in a treatment cycle of at least one day on-therapy (i.e., chemotherapy or radiotherapy)) followed by at least 3, 4, 5, 6, 7, 8, 9, 10, 11, 12, 13, 14 (or about 2 weeks), 15, 16, 17, 18, 19, 20, 21 (or about 3 weeks) days, or about 4 weeks (about one month) off-therapy (i.e., off chemo- or radio-therapy), the senolytic agent is administered on one or more days during the off-therapy time interval (time period) beginning on or after the second day of the off-therapy time interval and ending on or before the last day of the off-therapy time interval. By way of illustrative example, if n is the number of days off-therapy, then the senolytic agent is administered on at least one day and no more than n-1 days of the off-therapy time interval. In some embodiments when chemotherapy or radiotherapy is administered in a treatment cycle of at least one day on-therapy (i.e., chemotherapy or radiotherapy) followed by at least one week off-therapy, the senolytic agent is administered on one or more days during the off-therapy time interval beginning on or after the second day of the off-therapy time interval and ending on or before the last day of the off-therapy time interval.

A chemotherapy may be referred to as chemotherapy, chemotherapeutic, or chemotherapeutic drug. Many chemotherapeutics are compounds referred to as small organic molecules. Chemotherapy is a term that is also used to describe a combination of chemotherapeutic drugs that are administered to treat a particular cancer. As understood by a person skilled in the art, chemotherapy may also refer to a combination of two or more chemotherapeutic molecules that are administered coordinately and which may be referred to as combination chemotherapy. Numerous chemotherapeutic drugs are used in the oncology art and include, without limitation, alkylating agents; antimetabolites; anthracyclines, plant alkaloids; and topoisomerase inhibitors.

A cancer that may metastasize may be a solid tumor or may be a liquid tumor (e.g., a blood cancer, for example, leukemia). Cancers that are liquid tumors are classified in the art as those that occur in blood, bone marrow, and lymph nodes and include generally, leukemias (myeloid and lymphocytic), lymphomas (e.g., Hodgkin lymphoma), and melanoma (including multiple myeloma). Leukemias include for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors and occur in greater frequency in humans include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer (including squamous cell skin cancer), renal cancer, head and neck cancers, throat cancer, squamous carcinomas that form on the moist mucosal linings of the nose, mouth, throat, etc.), bladder cancer, osteosarcoma (bone cancer), cervical cancer, endometrial cancer, esophageal cancer, liver cancer, and kidney cancer. In certain specific embodiments, the senescent cell-associated disease or disorder treated or prevented (i.e., likelihood of occurrence or development is reduced) by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

The methods described herein are also useful for inhibiting, retarding or slowing progression of metastatic cancer of any one of the types of tumors described in the medical art. Types of cancers (tumors) include the following: adrenocortical carcinoma, childhood adrenocortical carcinoma, aids-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac (heart) tumors, cervical cancer, childhood cervical cancer, childhood chordoma, chronic myeloproliferative disorders, colon cancer, colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric (stomach) cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular (liver) cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (CML), hairy cell leukemia, lip cancer, liver cancer (primary), childhood liver cancer (primary), lobular carcinoma in situ (LCIS), lung cancer, non-small cell lung cancer, small cell lung cancer, lymphoma, aids-related lymphoma, Burkitt lymphoma, cutaneous T-cell lymphoma, Hodgkin lymphoma, non-Hodgkin lymphoma, primary central nervous system lymphoma (CNS), melanoma, childhood melanoma, intraocular (eye) melanoma, Merkel cell carcinoma, malignant mesothelioma, childhood malignant mesothelioma, metastatic squamous neck cancer with occult primary, midline tract carcinoma involving the NUT gene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleuropulmonary blastoma, prostate cancer, rectal cancer, renal pelvis transitional cell cancer, retinoblastoma, salivary gland cancer, childhood salivary gland cancer, Ewing sarcoma family of tumors, Kaposi Sarcoma, osteosarcoma, rhabdomyosarcoma, childhood rhabdomyosarcoma, soft tissue sarcoma, uterine sarcoma, Sezary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, and Waldenstrom macroglobulinemia.

### Chemotherapy and Radiotherapy Side Effects

In other embodiments, the senescence cell associated disorder or condition is a chemotherapeutic side effect or a radiotherapy side effect. Examples of chemotherapeutic agents that induce non-cancer cells to senesce include anthracyclines (such as doxorubicin, daunorubicin); taxols (e.g., paclitaxel); gemcitabine; pomalidomide; and lenalidomide. One or more of the senolytic agents administered as described herein may be used for treating and/or preventing (i.e., reducing the likelihood or occurrence of) a chemotherapeutic side effect or a radiotherapy side effect. Removal or destruction of senescent cells may ameliorate acute toxicity, including acute toxicity comprising energy imbalance, of a chemotherapy or radiotherapy. Acute toxic side effects include but are not limited to gastrointestinal toxicity (e.g., nausea, vomiting, constipation, anorexia, diarrhea), peripheral neuropathy, fatigue, malaise, low physical activity, hematological toxicity (e.g., anemia), hepatotoxicity, alopecia (hair loss), pain, infection, mucositis, fluid retention, dermatological toxicity (e.g., rashes, dermatitis, hyperpigmentation, urticaria, photosensitivity, nail changes), mouth (e.g., oral mucositis), gum or throat problems, or any toxic side effect caused by a chemotherapy or radiotherapy. For example, toxic side effects caused by radiotherapy or chemotherapy may be ameliorated by the methods described herein. Accordingly, in certain embodiments, methods are provided herein for ameliorating (reducing, inhibiting, or preventing occurrence (i.e., reducing the likelihood of occurrence)) acute toxicity or reducing severity of a toxic side effect (i.e., deleterious side effect) of a chemotherapy or radiotherapy or both in a subject who receives the therapy, wherein the method comprises administering to the subject an agent that selectively kills, removes, or destroys or facilitates selective destruction of senescent cells. Administration of senolytic agents described herein for treating or reducing the likelihood of occurrence or reducing the severity of a chemotherapy or radiotherapy side effect may be accomplished by the same treatment courses described above for treatment/prevention of metastasis. As described for treating or preventing (i.e., reducing the likelihood of occurrence of) metastasis, the senolytic agent is administered during the off-chemotherapy or off-radiotherapy time interval or after the chemotherapy or radiotherapy treatment regimen has been completed.

In more specific embodiments, the acute toxicity is an acute toxicity comprising energy imbalance and may comprise one or more weight loss, endocrine change(s) (e.g., hormone imbalance, change in hormone signaling), and change(s) in body composition. In certain embodiments, an acute toxicity comprising energy imbalance relates to decreased or reduced ability of the subject to be physically active, as indicated by decreased or diminished expenditure of energy than would be observed in a subject who did not receive the medical therapy. By way of non-limiting example, such an acute toxic effect that comprises energy imbalance includes low physical activity. In other embodiments, energy imbalance comprises fatigue or malaise.

In some embodiments, a chemotherapy side effect to be treated or prevented (i.e., likelihood of occurrence is reduced) by a senolytic agent described herein is cardiotoxicity. A subject who has cancer that is being treated with an anthracycline (such as doxorubicin, daunorubicin) may be treated with one or more senolytic agents described herein that reduce, ameliorate, or decrease the cardiotoxicity of the anthracycline. As is well understood in the medical art, because of the cardiotoxicity associated with anthracyclines, the maximum lifetime dose that a subject can receive is limited even if the cancer is responsive to the drug. Administration of one or more of the senolytic agents may reduce the cardiotoxicity such that additional amounts of the anthracycline can be administered to the subject, resulting in an improved prognosis related to cancer disease. In some embodiments, cardiotoxicity results from administration of an anthracycline, such as doxorubicin. Doxorubicin is an anthracycline topoisomerase inhibitor that is approved for treating patients who have ovarian cancer after failure of a platinum-based therapy; Kaposi's sarcoma after failure of primary systemic chemotherapy or intolerance to the therapy; or multiple myeloma in combination with bortezomib in patients who have not previously received bortezomib or who have received at least one prior therapy. Doxorubicin may cause myocardial damage that could lead to congestive heart failure if the total lifetime dose to a patient exceeds 550 mg/m2. Cardiotoxicity may occur at even lower doses if the patient also receives mediastinal irradiation or another cardiotoxic drug.

In other embodiments, a senolytic agent described herein may be used in the methods as provided herein for ameliorating chronic or long-term side effects. Chronic toxic side effects typically result from multiple exposures to or administrations of chemotherapy or radiotherapy over a longer period of time. Certain toxic effects appear long after treatment (also called late toxic effects) and result from damage to an organ or system by the therapy. Organ dysfunction (e.g., neurological, pulmonary, cardiovascular, and endocrine dysfunction) has been observed in patients who were treated for cancers during childhood (see, e.g., Hudson et al., JAMA 309 92013)₂₃₇₁-2381). Without wishing to be bound by any particular theory, by destroying senescent cells, particular normal cells that have been induced to senescence by chemotherapy or radiotherapy, the likelihood of occurrence of a chronic side effect may be reduced, or the severity of a chronic side effect may be reduced or diminished, or the time of onset of a chronic side effect may be delayed. Chronic and/or late toxic side effects that occur in subjects who received chemotherapy or radiation therapy include by way of non-limiting example, cardiomyopathy, congestive heart disease, inflammation, early menopause, osteoporosis, infertility, impaired cognitive function, peripheral neuropathy, secondary cancers, cataracts and other vision problems, hearing loss, chronic fatigue, reduced lung capacity, and lung disease.

In addition, by killing or removing senescent cells in a subject who has a cancer by administering a senolytic agent, the sensitivity to the chemotherapy or the radiotherapy may be enhanced in a clinically or statistically significant manner than if the senolytic agent was not administered. In other words, development of chemotherapy or radiotherapy resistance may be inhibited when a senolytic agent is administered to a subject treated with the respective chemotherapy or radiotherapy.

### Age-Related Diseases and Disorders

A senolytic agent described herein selectively kills senescent cells. In this way, targeting senescent cells during the course of aging may be a preventative strategy. Accordingly, administration of a senolytic agent described herein to a subject may prevent comorbidity and delay mortality in an older subject. Further, selective killing of senescent cells may boost the immune system, extend the health span, and improve the quality of life in a subject.

A senolytic agent may also be useful for treating or preventing (i.e., reducing the likelihood of occurrence) of an age-related disease or disorder that occurs as part of the natural aging process or that occurs when the subject is exposed to a senescence inducing agent or factor (e.g., irradiation, chemotherapy, smoking tobacco, high fat/high sugar diet, other environmental factors). An age-related disorder or disease or an age-sensitive trait may be associated with a senescence-inducing stimulus. The efficacy of a method of treatment described herein may be manifested by reducing the number of symptoms of an age-related disorder or age-sensitive trait associated with a senescence-inducing stimulus, decreasing the severity of one or more symptoms, or delaying the progression of an age-related disorder or age-sensitive trait associated with a senescence-inducing stimulus. In other embodiments, preventing an age-related disorder or age-sensitive trait associated with a senescence-inducing stimulus refers to preventing (i.e., reducing the likelihood of occurrence) or delaying onset of an age-related disorder or age-sensitive trait associated with a senescence-inducing stimulus, or reoccurrence of one or more age-related disorder or age-sensitive trait associated with a senescence-inducing stimulus. Age related diseases or conditions include, for example, renal dysfunction, kyphosis, herniated intervertebral disc, frailty, hair loss, hearing loss, vision loss (blindness or impaired vision), muscle fatigue, skin conditions, skin nevi, diabetes, metabolic syndrome, and sarcopenia. Vision loss refers to the absence of vision when a subject previously had vision. Various scales have been developed to describe the extent of vision and vision loss based on visual acuity. Age-related diseases and conditions also include dermatological conditions, for example without limitation, treating one or more of the following conditions: wrinkles, including superficial fine wrinkles; hyperpigmentation; scars; keloid; dermatitis; psoriasis; eczema (including seborrheic eczema); rosacea; vitiligo; ichthyosis vulgaris; dermatomyositis; and actinic keratosis. Frailty has been defined as a clinically recognizable state of increased vulnerability resulting from aging-associated decline in reserve and function across multiple physiologic systems that compromise a subject's ability to cope with every day or acute stressors. Frailty may be characterized by compromised energetics characteristics such as low grip strength, low energy, slowed walking speed, low physical activity, and/or unintentional weight loss. Studies have suggested that a patient may be diagnosed with frailty when three of five of the foregoing characteristics are observed (see, e.g., Fried et al., J. Gerontol. A Biol. Sci. Med, Sci. 56 (3) (2001) M146-M156; Xue, Clin. Geriatr. Med. 27 (1) (2001) 1-15). In certain embodiments, aging and diseases and disorders related to aging may be treated or prevented (i.e., the likelihood of occurrence of is reduced) by administering a senolytic agent. The senolytic agent may inhibit senescence of adult stem cells or inhibit accumulation, kill, or facilitate removal of adult stem cells that have become senescent. The importance of preventing senescence in stem cells to maintain regenerative capacity of tissues is discussed, e.g., in Park et al., J. Clin. Invest. 113 (2004) 175-179; and Sousa-Victor, Nature 506 (2014) 316-321.

Methods of measuring aging are known in the art. For example, aging may be measured in the bone by incident non-vertebral fractures, incident hip fractures, incident total fractures, incident vertebral fractures, incident repeat fractures, functional recovery after fracture, bone mineral density decrease at the lumbar spine and hip, rate of knee buckling, NSAID use, number of joints with pain, and osteoarthritis. Aging may also be measured in the muscle by functional decline, rate of falls, reaction time and grip strength, muscle mass decrease at upper and lower extremities, and dual tasking 10-meter gait speed. Further, aging may be measured in the cardiovascular system by systolic and diastolic blood pressure change, incident hypertension, major cardiovascular events such as myocardial infarction, stroke, congestive heart disease, and cardiovascular mortality. Additionally, aging may be measured in the brain by cognitive decline, incident depression, and incident dementia. Also, aging may be measured in the immune system by rate of infection, rate of upper respiratory infections, rate of flu-like illness, incident severe infections that lead to hospital admission, incident cancer, rate of implant infections, and rate of gastrointestinal infections. Other indications of aging may include, but not limited to, decline in oral health, tooth loss, rate of GI symptoms, change in fasting glucose and/or insulin levels, body composition, decline in kidney function, quality of life, incident disability regarding activities of daily living, and incident nursing home admission. Methods of measuring skin aging are known in the art and may include trans-epidermal water loss (TEWL), skin hydration, skin elasticity, area ratio analysis of crow's feet, sensitivity, radiance, roughness, spots, laxity, skin tone homogeneity, softness, and relief (variations in depth).

Administration of a senolytic agent described herein can prolong prolonging survival when compared to expected survival if a subject were not receiving treatment. Subjects in need of treatment include those who already have the disease or disorder as well as subjects prone to have or at risk of developing the disease or disorder, and those in which the disease, condition, or disorder is to be treated prophylactically. A subject may have a genetic predisposition for developing a disease or disorder that would benefit from clearance of senescent cells or may be of a certain age wherein receiving a senolytic agent would provide clinical benefit to delay development or reduce severity of a disease, including an age-related disease or disorder.

In other embodiments, a method is provided for treating a senescence-associated disease or disorder that further comprises identifying a subject who would benefit from treatment with a senolytic agent described herein (i.e., phenotyping; individualized treatment). This method comprises first detecting the level of senescent cells in the subject, such as in a particular organ or tissue of the subject. A biological sample may be obtained from the subject, for example, a blood sample, serum or plasma sample, biopsy specimen, body fluids (e.g., lung lavage, ascites, mucosal washings, synovial fluid, vitreous fluid, spinal fluid), bone marrow, lymph nodes, tissue explant, organ culture, or any other tissue or cell preparation from a subject. The level of senescent cells may be determined according to any of the in vitro assays or techniques described herein. For example, senescent cells may be detected by morphology (as viewed by microscopy, for example); production of senescence associated markers such as, senescence-associated-galactosidase (SA-β-gal), p16INK4a, p21, PAI-1, or any one or more SASP factors (e.g., IL-6, MMP3). The senescent cells and non-senescent cells of the biological sample may also be used in an in vitro cell assay in which the cells are exposed to any one of the senolytic agents described herein to determine the capability of the senolytic agent to kill the subject's senescent cells without undesired toxicity to non-senescent cells. In addition, these methods may be used to monitor the level of senescent cells in the subject before, during, and after treatment with a senolytic agent. In certain embodiments, the presence of senescent cells may be detected (e.g., by determining the level of a senescent cell marker expression of mRNA, for example), and the treatment course and/or non-treatment interval can be adjusted accordingly.

### Methods of Treatment and Prevention of GPX4 Diseases and Disorders

Methods of treating, preventing, or ameliorating symptoms of a glutathione peroxidase 4 (GPX4)-associated disease in a subject, comprises administering an effective amount of one or more compounds disclosed herein or the compositions disclosed herein are provided. In some embodiments, the GPX4-associated disease is cancer, neurotic disorder, neurodegenerative disorder, spondylometaphyseal dysplasia, mixed cerebral palsy, pontocerebellar hypoplasia or male infertility.

In some embodiments, the GPX4-associated disease is cancer. Non limiting examples of cancer include hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma or non-small cell lung carcinoma. In some embodiments, the cancer is hepatocellular carcinoma. In other embodiments, the cancer is metastatic. In still other embodiments, the cancer is hypersensitive to ferroptosis. In still other embodiments, the cancer is refractory to standard cancer treatment. In still other embodiments, the cancer has mesenchymal features. In still other embodiments, the cancer is a multiple therapy resistant cancer.

Also provided herein is a method for modulating the activity of GPX4 in a subject comprising administering an effective amount of one or more compounds disclosed herein or the compositions disclosed herein. In some embodiments, the modulation comprises inhibiting GPX4 activity.

Also provided herein is a method for increasing the level of peroxide in a subject comprising administering an effective amount of one or more compounds disclosed herein or the compositions disclosed herein. Non-limiting examples of peroxide include hydrogen peroxide, organic hydroperoxide, lipid peroxide, and combinations thereof.

Also provided herein is a method for inducing ferroptosis in a cell, comprising contacting the cell with an effective amount of one or more compounds disclosed herein or the compositions disclosed herein. The cell may have aberrant lipid accumulation. In some embodiments, the cell is a cancer cell which includes but is not limited to hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, or non-small cell lung carcinoma cell. In other embodiments, the cancer cell is a hepatocellular carcinoma cell.

In some embodiments, the cancer cell is metastatic. In other embodiments, the cancer cell is hypersensitive to ferroptosis. In still other embodiments, the cancer has mesenchymal features. In still other embodiments the cancer is a multiple therapy resistant cancer.

The hypersensitivity to ferropotosis may be identified by NADPH abundance, GCH1 expression, NF2-YAP activity, EMT signature, and GPX4 expression. In some embodiments, the cancer cell is selected from the group consisting of hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma or non-small cell lung carcinoma cell. In some embodiments, the cancer is a hepatocellular carcinoma cell.

### Pharmaceutical Compositions and Methods of Administration

Also provided herein are pharmaceutical compositions that comprise a senolytic agent as described herein and at least one pharmaceutically acceptable excipient, which may also be called a pharmaceutically suitable excipient or carrier (i.e., a non-toxic material that does not interfere with the activity of the active ingredient). A pharmaceutical composition may be a sterile aqueous or non-aqueous solution, suspension or emulsion (e.g., a microemulsion). The excipients described herein are examples and are in no way limiting. An effective amount or therapeutically effective amount refers to an amount of one or more senolytic agents administered to a subject, either as a single dose or as part of a series of doses, which is effective to produce a desired therapeutic effect.

When two or more senolytic agents are administered to a subject for treatment of a disease or disorder described herein, each of the senolytic agents may be formulated into separate pharmaceutical compositions. A pharmaceutical preparation may be prepared that comprises each of the separate pharmaceutical compositions (which may be referred to for convenience, for example, as a first pharmaceutical composition and a second pharmaceutical composition comprising each of the first and second senolytic agents, respectively). Each of the pharmaceutical compositions in the preparation may be administered at the same time (i.e., concurrently) and via the same route of administration or may be administered at different times by the same or different administration routes. Alternatively, two or more senolytic agents may be formulated together in a single pharmaceutical composition.

In other embodiments, a combination of at least one senolytic agent and at least one inhibitor of an mTOR, NF-DB, or PI3K pathway may be administered to a subject in need thereof. When at least one senolytic agent and an inhibitor of one or more of mTOR, NF-DB, or PI3K pathways are both used together in the methods described herein for selectively killing senescent cells, each of the agents may be formulated into the same pharmaceutical composition or formulated in separate pharmaceutical compositions. A pharmaceutical preparation may be prepared that comprises each of the separate pharmaceutical compositions, which may be referred to for convenience, for example, as a first pharmaceutical composition and a second pharmaceutical composition comprising each of the senolytic agent and the inhibitor of one or more of mTOR, NF-DB, or PI3K pathways, respectively. Each of the pharmaceutical compositions in the preparation may be administered at the same time and via the same route of administration or may be administered at different times by the same or different administration routes.

Pharmacokinetics of a senolytic agent (or one or more metabolites thereof) that is administered to a subject may be monitored by determining the level of the senolytic agent in a biological fluid, for example, in the blood, blood fraction (e.g., serum), and/or in the urine, and/or other biological sample or biological tissue from the subject. Any method practiced in the art and described herein to detect the agent may be used to measure the level of the senolytic agent during a treatment course.

The dose of a senolytic agent described herein for treating a senescence cell associated disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of the senolytic agent for treating a senescence-associated disease or disorder, suitable duration and frequency of administration of the senolytic agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a senolytic agent (including when administered for prophylactic benefit) described herein are well within the skill of a person skilled in the relevant art. When two or more senolytic agents are administered to treat a senescence-associated disease or disorder, the optimal dose of each senolytic agent may be different, such as less, than when either agent is administered alone as a single agent therapy. In certain embodiments, two senolytic agents in combination act synergistically or additively, and either agent may be used in a lesser amount than if administered alone. An amount of a senolytic agent that may be administered per day may be, for example, between about 0.01 mg/kg and 100 mg/kg (e.g., between about 0.1 to 1 mg/kg, between about 1 to 10 mg/kg, between about 10-50 mg/kg, between about 50-100 mg/kg body weight. In other embodiments, the amount of a senolytic agent that may be administered per day is between about 0.01 mg/kg and 1000 mg/kg, between about 100-500 mg/kg, or between about 500-1000 mg/kg body weight. The optimal dose (per day or per course of treatment) may be different for the senescence-associated disease or disorder to be treated and may also vary with the administrative route and therapeutic regimen.

Pharmaceutical compositions comprising a senolytic agent can be formulated in a manner appropriate for the delivery method by using techniques routinely practiced in the art. The composition may be in the form of a solid (e.g., tablet, capsule), semi-solid (e.g., gel), liquid, or gas (aerosol). In other certain specific embodiments, the senolytic agent (or pharmaceutical composition comprising same) is administered as a bolus infusion. In certain embodiments when the senolytic agent is delivered by infusion, the senolytic agent is delivered to an organ or tissue comprising senescent cells to be killed via a blood vessel in accordance with techniques routinely performed by a person skilled in the medical art.

Pharmaceutical acceptable excipients are well known in the pharmaceutical art and described, for example, in Rowe et al., Handbook of Pharmaceutical Excipients: A Comprehensive Guide to Uses, Properties, and Safety, 5th Ed., 2006, and in Remington: The Science and Practice of Pharmacy (Gennaro, 21st Ed. Mack Pub. Co., Easton, Pa. (2005)). Exemplary pharmaceutically acceptable excipients include sterile saline and phosphate buffered saline at physiological pH. Preservatives, stabilizers, dyes, buffers, and the like may be provided in pharmaceutical composition. In addition, antioxidants and suspending agents may also be used. In general, the type of excipient is selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the agent(s) of the composition upon administration. In other embodiments, the agent may be encapsulated within liposomes using technology known and practiced in the art. Pharmaceutical compositions may be formulated for any appropriate manner of administration described herein and, in the art.,

A pharmaceutical composition may be delivered to a subject in need thereof by any one of several routes known to a person skilled in the art. By way of non-limiting example, the composition may be delivered orally, intravenously, intraperitoneally, by infusion (e.g., a bolus infusion), subcutaneously, enteral, rectal, intranasal, by inhalation, buccal, sublingual, intramuscular, transdermal, intradermal, topically, intraocular, vaginal, rectal, or by intracranial injection, or any combination thereof. In certain embodiments, administration of a dose, as described above, is via intravenous, intraperitoneal, directly into the target tissue or organ, or subcutaneous route. In certain embodiments, a delivery method includes drug-coated or permeated stents for which the drug is the senolytic agent. Formulations suitable for such delivery methods are described in greater detail herein.

In certain embodiments, a senolytic agent (which may be combined with at least one pharmaceutically acceptable excipient to form a pharmaceutical composition) is administered directly to the target tissue or organ comprising senescent cells that contribute to the manifestation of the disease or disorder. In specific embodiments when treating osteoarthritis, the at least one senolytic agent is administered directly to an osteoarthritic joint (i.e., intra-articularly) of a subject in need thereof. In other specific embodiments, a senolytic agent(s) may be administered to the joint via topical, transdermal, intradermal, or subcutaneous route. In other certain embodiments, methods are provided herein for treating a cardiovascular disease or disorder associated with arteriosclerosis, such as atherosclerosis by administering directly into an artery. In other embodiments, a senolytic agent (which may be combined with at least one pharmaceutically acceptable excipient to form a pharmaceutical composition) for treating a senescent-associated pulmonary disease or disorder may be administered by inhalation, intranasally, by intubation, or intrathecally, for example, to provide the senolytic agent more directly to the affected pulmonary tissue. By way of another non-limiting example, the senolytic agent (or pharmaceutical composition comprising the senolytic agent) may be delivered directly to the eye either by injection (e.g., intraocular or intravitreal) or by conjunctival application underneath an eyelid of a cream, ointment, gel, or eye drops. In more particular embodiments, the senolytic agent or pharmaceutical composition comprising the senolytic agent may be formulated as a timed release (also called sustained release, controlled release) composition or may be administered as a bolus infusion.

A pharmaceutical composition (e.g., for oral administration or for injection, infusion, subcutaneous delivery, intramuscular delivery, intraperitoneal delivery or other method) may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In other embodiments, for treatment of an ophthalmological condition or disease, a liquid pharmaceutical composition may be applied to the eye in the form of eye drops. A liquid pharmaceutical composition may be delivered orally.

For oral formulations, at least one of the senolytic agents described herein can be used alone or in combination with appropriate additives to make tablets, powders, granules or capsules, and if desired, with diluents, buffering agents, moistening agents, preservatives, coloring agents, and flavoring agents. The compounds may be formulated with a buffering agent to provide protection of the compound from low pH of the gastric environment and/or an enteric coating. A senolytic agent included in a pharmaceutical composition may be formulated for oral delivery with a flavoring agent, e.g., in a liquid, solid or semi-solid formulation and/or with an enteric coating.

A pharmaceutical composition comprising any one of the senolytic agents described herein may be formulated for sustained or slow release (also called timed release or controlled release). Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible and may also be biodegradable; preferably the formulation provides a relatively constant level of active component release. The amount of active agent contained within a sustained release formulation depends upon the site of implantation, the rate and expected duration of release, and the nature of the condition, disease or disorder to be treated or prevented.

In certain embodiments, the pharmaceutical compositions comprising a senolytic agent are formulated for transdermal, intradermal, or topical administration. The compositions can be administered using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered topically or injected directly into the skin adjacent to or within the area to be treated (intradermally or subcutaneously). The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, thimerosal, and combinations thereof.

Pharmaceutical compositions comprising a senolytic agent can be formulated as emulsions for topical application. An emulsion contains one liquid distributed to the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in compositions, patches, bandages and articles. These systems are semisolid emulsions, micro-emulsions, or foam emulsion systems.

Controlled or sustained release transdermal or topical formulations can be achieved by the addition of time-release additives, such as polymeric structures, matrices, which are available in the art. For example, the compositions may be administered through use of hot-melt extrusion articles, such as bioadhesive hot-melt extruded film. The formulation can comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the compound.

An insert, transdermal patch, bandage or article can comprise a mixture or coating of polymers that provide release of the active agents at a constant rate over a prolonged period of time. In some embodiments, the article, transdermal patch or insert comprises water-soluble pore forming agents, such as polyethylene glycol (PEG) that can be mixed with water insoluble polymers to increase the durability of the insert and to prolong the release of the active ingredients.

A polymer formulation can also be utilized to provide controlled or sustained release. Bioadhesive polymers described in the art may be used. By way of example, a sustained-release gel and the compound may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. The polymer can also be in the form of a coating or part of a bandage, stent, catheter, vascular graft, or other device to facilitate delivery of the senolytic agent. The matrices can be formed by solvent evaporation, spray drying, solvent extraction and other methods known to those skilled in the art.

Kits with unit doses of one or more of the agents described herein, usually in oral or injectable doses, are provided. Such kits may include a container containing the unit dose, an informational package insert describing the use and attendant benefits of the drugs in treating the senescent cell associated disease, and optionally an appliance or device for delivery of the composition.

### Combination Therapy

The compounds and compositions disclosed herein may also be used in combination with one or more other active ingredients. In certain aspects, the compounds may be administered in combination, or sequentially, with another therapeutic agent. Such other therapeutic agents include those known for treatment, prevention, or amelioration of one or more symptoms disclosed herein. Many such therapeutic agents are known in the art.

It should be understood that any suitable combination of the compounds and compositions provided herein with one or more of the above therapeutic agents and optionally one or more further pharmacologically active substances are considered to be within the scope of the present disclosure. In some aspects, the compounds and compositions provided herein are administered prior to or subsequent to the one or more additional active ingredients.

Examples of compounds which may be administered with the compounds disclosed herein include, but are not limited to, dasatinib, quercetin, fisetin, leeutolin, curcumin, curcumin analog EF24, Navioclax (ABT253), A1331852, A1155463, Geldamycin, Tanespimycin, Alvespimycin, piperlongumeine, FOXO-4 peptide, Nutlin3a, cardiac glycosides (e.g., Ouabain, Proscillaridin A, Digoxin, etc.), HSP-90 inhibitors, triptolide, EF-24, Procyanidin C₁, Azithromycin, Roxithromycin, 25-hydroxycholesterol, SSK1, BIRC5 knockout, BCL-2 inhibitors, Src inhibitors, PD-1, CTLA-4 ipilimumab, pembrolizumab, nivolumab, atezolizumab, avelumab, durvalumab, cemiplimab, ofatumumab, blinatumomab, daratumumab, elotuzumab, obinutuzumab, talimogene laherparepvec, necitumumab, lenalidomide, dinutuximab and combinations thereof.

Finally, it should be noted that there are alternative ways of implementing the present invention. Accordingly, the present aspects are to be considered as illustrative and not restrictive, and the invention is not to be limited to the details given herein but may be modified within the scope and equivalents of the appended claims.

All publications and patents cited herein are incorporated by reference in their entirety.

The following examples are provided for illustrative purposes only and are not intended to limit the scope of the invention.

### Examples

Scheme 1 illustrates the preparation of ethyl(3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinate (1)

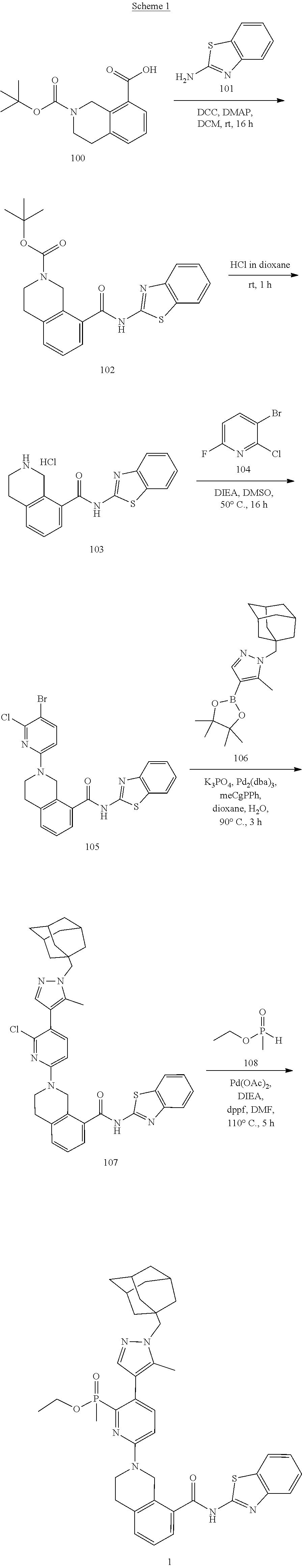

### tert-butyl 8-(benzo[d]thiazol-2-ylcarbamoyl)-3,4-dihydroisoquinoline-2 (1H)-carboxylate (102)

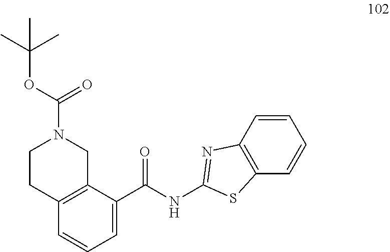 [SMILES: CC(C)(C)OC(=O)N1CCc2cccc(C(=O)Nc3nc4ccccc4s3)c2C1]

To a stirred solution of 2-(tert-butoxycarbonyl)-1,2,3,4-tetrahydroisoquinoline-8-carboxylic acid (6.6 g, 23.80 mmol) (100) in DCM (100 mL) were added benzo[d]thiazol-2-amine (5.36 g, 35.70 mmol) (101), DCC(7.37 g, 35.70 mmol) and DMAP (8.72 g, 71.40 mmol). The reaction was stirred at room temperature for 16 h. The reaction mixture was quenched with water (200 mL) and extracted with DCM (3×150 mL). The combined organic layers were washed with water (100 mL) and brine (100 mL), dried over anhydrous sodium sulfate and concentrated under vacuum. The residue was purified by column chromatography on silica gel eluting with ethyl acetate in petroleum ether (0 to 30%) to afford tert-butyl 8-(benzo[d]thiazol-2-ylcarbamoyl)-3,4-dihydroisoquinoline-2 (1H)-carboxylate (7.1 g, 72%) (102) as a white solid. 1H NMR (400 MHZ, DMSO-d₆) δ 12.84 (s, 1H), 8.03 (d, J=7.2 Hz, 1H), 7.79 (d, J=8.0 Hz, 1H), 7.59 (dd, J=1.6, 7.4 Hz, 1H), 7.51-7.43 (m, 1H), 7.42-7.29 (m, 3H), 4.70 (s, 2H), 3.65-3.49 (m, 2H), 2.88 (t, J=6.0 Hz, 2H), 1.38 (s, 9H). ESI-MS [M+H]⁺calcd for (C₂₂H23N₃O₃S): 410.15; found: 410.25.

### N-(benzo[d]thiazol-2-yl)-1,2,3,4-tetrahydroisoquinoline-8-carboxamide hydrochloride (103)

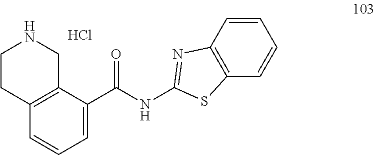 [SMILES: O=C(Nc1nc2ccccc2s1)c1cccc2c1CNCC2 ; Cl]

A mixture of tert-butyl 8-(benzo[d]thiazol-2-ylcarbamoyl)-3,4-dihydroisoquinoline-2 (1H)-carboxylate (7.4 g, 18.07 mmol) (102) and 4 M HCl in 1,4-dioxane (80 mL) was stirred at room temperature for 1 h. The reaction mixture was concentrated under vacuum to give N-(benzo[d]thiazol-2-yl)-1,2,3,4-tetrahydroisoquinoline-8-carboxamide hydrochloride (6.9 g, crude) (103) as a white solid. ¹H NMR (400 MHZ, DMSO-d₆) δ 9.79 (s, 2H), 8.02 (d, J=7.6 Hz, 1H), 7.79 (d, J=8.0 Hz, 1H), 7.75 (dd, J=2.1, 6.9 Hz, 1H), 7.51-7.41 (m, 3H), 7.39-7.31 (m, 1H), 4.51-4.40 (m, 2H), 3.41-3.30 (m, 2H), 3.11 (t, J=6.0 Hz, 2H). ESI-MS [M+H]⁺calcd for (C₁₇H₁₅N₃OS): 310.09; found: 310.10.

### N-(benzo[d]thiazol-2-yl)-2-(5-bromo-6-chloropyridin-2-yl)-1,2,3,4-tetrahydroisoquinoline-8-carboxamide (105)

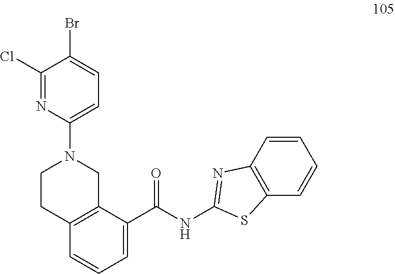 [SMILES: O=C(Nc1nc2ccccc2s1)c1cccc2c1CN(c1ccc(Br)c(Cl)n1)CC2]

To a solution of N-(benzo[d]thiazol-2-yl)-1,2,3,4-tetrahydroisoquinoline-8-carboxamide hydrochloride (2.5 g, 5.79 mmol) (103) in DMSO (80 mL) was added 3-bromo-2-chloro-6-fluoropyridine (1.5 g, 7.14 mmol) (104) and DIEA (5.0 g, 38.75 mmol). The reaction mixture was stirred at 50° C. for 16 h. The cooled reaction mixture was poured into ice-water (300 mL) and stirred for 10 min. The precipitate was collected by filtration and slurred in methanol (60 mL). The solid was collected by filtration and dried under reduced pressure to afford N-(benzo[d]thiazol-2-yl)-2-(5-bromo-6-chloropyridin-2-yl)-1,2,3,4-tetrahydroisoquinoline-8-carboxamide (2.8 g, 77%) (105) as a yellow solid. ¹H NMR (400 MHZ, DMSO-d₆) δ 12.87 (s, 1H), 8.04 (d, J=7.6 Hz, 1H), 7.87-7.74 (m, 2H), 7.63 (d, J=7.2 Hz, 1H), 7.51-7.40 (m, 2H), 7.40-7.31 (m, 2H), 6.75 (d, J=8.8 Hz, 1H), 4.90 (s, 2H), 3.78 (t, J=6.0 Hz, 2H), 2.99 (t, J=5.6 Hz, 2H). ESI-MS [M+H]⁺calcd for (C₂₂H16BrClN₄OS): 498.99, 500.99; found: 499.00, 501.00.

### 2-(5-(1-(((3r,5r,7r)-adamantan-1-yl)methyl)-5-methyl-1H-pyrazol-4-yl)-6-chloropyridin-2-yl)-N-(benzo[d]thiazol-2-yl)-1,2,3,4-tetrahydroisoquinoline-8-carboxamide (107)

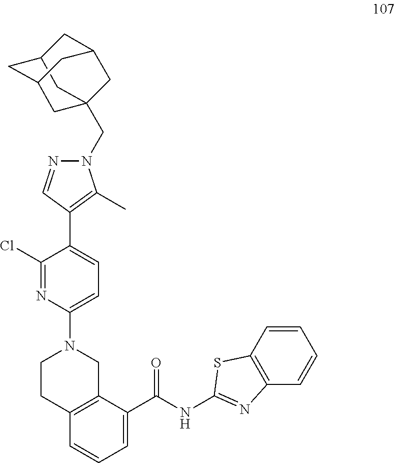 [SMILES: Cc1c(-c2ccc(N3CCc4cccc(C(=O)Nc5nc6ccccc6s5)c4C3)nc2Cl)cnn1CC12CC3CC(CC(C3)C1)C2]

A mixture of N-(benzo[d]thiazol-2-yl)-2-(5-bromo-6-chloropyridin-2-yl)-1,2,3,4-tetrahydroisoquinoline-8-carboxamide (1.60 g, 3.21 mmol) (105), 1-(((3r,5r,7r)-adamantan-1-yl) methyl)-5-methyl-4-(4,4,5,5-tetramethyl-1,3,2-dioxaborolan-2-yl)-1H-pyrazole (1.0 g, 2.81 mmol) (106), Pd₂(dba)₃ (257 mg, 0.28 mmol), meCgPPh (164 mg, 0.56 mmol) and K₃PO₄ (1.79 g, 8.86 mmol) in dioxane (36 mL) and water (6 mL) was degassed and backfilled with nitrogen for five times. The reaction mixture was heated at 90° C. for 3 h. The cooled reaction mixture was quenched with water (100 mL) and extracted with ethyl acetate (3×80 mL). The combined organic layers was washed with brine (80 mL), dried over sodium sulfate and concentrated under vacuum. The residue was slurred in ethyl acetate and petroleum ether (1:1) (20 mL). The solid was collected by filtration and dried under reduced pressure to afford 2-(5-(1-(((3r,5r,7r)-adamantan-1-yl)methyl)-5-methyl-1H-pyrazol-4-yl)-6-chloropyridin-2-yl)-N-(benzo[d]thiazol-2-yl)-1,2,3,4-tetrahydroisoquinoline-8-carboxamide (1.3 g, 71%) (107) as a yellow solid. ¹H NMR (400 MHz, DMSO-d₆) δ 12.86 (brs, 1H), 8.02 (d, J=7.6 Hz, 1H), 7.77 (d, J=8.0 Hz, 1H), 7.63 (d, J=7.8 Hz, 1H), 7.51 (d, J=8.4 Hz, 1H), 7.49-7.41 (m, 2H), 7.40-7.30 (m, 3H), 6.81 (d, J=8.8 Hz, 1H), 4.94 (s, 2H), 3.82 (t, J=6.0 Hz, 2H), 3.72 (s, 2H), 3.01 (t, J=5.6 Hz, 2H), 2.12 (s, 3H), 1.92 (s, 3H), 1.70-1.60 (m, 3H), 1.60-1.49 (m, 9H). ESI-MS [M+H]⁺calcd for (C₃₇H₃₇ClN₆OS): δ 49.24, 651.24; found: 649.55, 651.55.

### Example 1: ethyl(3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinate (1)

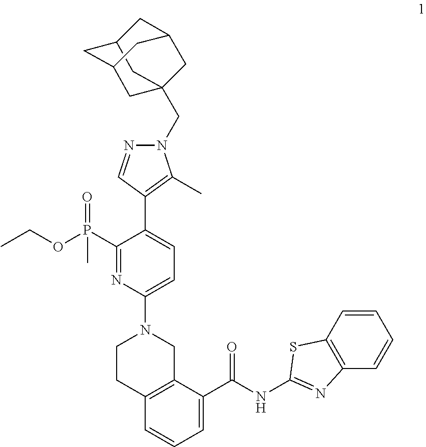 [SMILES: CCOP(C)(=O)c1nc(N2CCc3cccc(C(=O)Nc4nc5ccccc5s4)c3C2)ccc1-c1cnn(CC23CC4CC(CC(C4)C2)C3)c1C]

To a mixture of 2-(5-(1-(((3r,5r,7r)-adamantan-1-yl)methyl)-5-methyl-1H-pyrazol-4-yl)-6-chloropyridin-2-yl)-N-(benzo[d]thiazol-2-yl)-1,2,3,4-tetrahydroisoquinoline-8-carboxamide (1.2 g, 1.85 mmol) (107), Pd(OAc)₂ (83 mg, 0.37 mmol), DIEA (955 mg, 7.40 mmol) and dppf (227 mg, 0.41 mmol) in DMF (40 mL) was added ethyl methylphosphinate (999 mg, 9.25 mmol) (108). The reaction mixture was degassed and backfilled with nitrogen five times and heated at 110° C. for 3 h. The cooled reaction mixture was quenched with water (100 mL) and extracted with ethyl acetate (4×80 mL). The combined organic layers was washed with brine (100 mL), dried over sodium sulfate and concentrated under vacuum. The residue was purified by column chromatography on silica gel eluting with ethyl acetate in petroleum ether (0 to 70%) to afford ethyl(3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinate (860 mg, 64%) (1) as a light-yellow solid. 80 mg of ethyl(3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinate (1) was further purified by Prep-HPLC with the following conditions: Column: XBridge Prep OBD C18 Column, 30×150 mm, 5 um; Mobile Phase A: Water (10 mmol/L NH₄HCO₃), Mobile Phase B: Acetonitrile; Flow rate: 60 mL/min; Gradient: 62% B to 92% B in 13 min; Wave Length: 254 nm/220 nm; RT (min): 8.33 to afford ethyl(3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinate (26.0 mg, 32%) (1) as a white solid. ¹H NMR (400 MHZ, DMSO-d₆) δ 12.86 (brs, 1H), 8.03 (d, J=7.6 Hz, 1H), 7.78 (d, J=8.0 Hz, 1H), 7.59 (d, J=6.8 Hz, 1H), 7.51-7.30 (m, 6H), 7.02 (d, J=8.8 Hz, 1H), 5.10-4.92 (m, 2H), 3.89-3.77 (m, 2H), 3.76-3.65 (m, 3H), 3.63-3.56 (m, 1H), 3.10-2.98 (m, 2H), 2.09 (s, 3H), 1.91 (s, 3H), 1.72-1.60 (m, 3H), 1.53 (s, 9H), 1.41 (d, J=14.4 Hz, 3H), 0.97-0.86 (m, 3H). ESI-MS [M+H]⁺calcd for (C₄₀H₄₅N₆O₃PS): 721.30; found: 721.45.

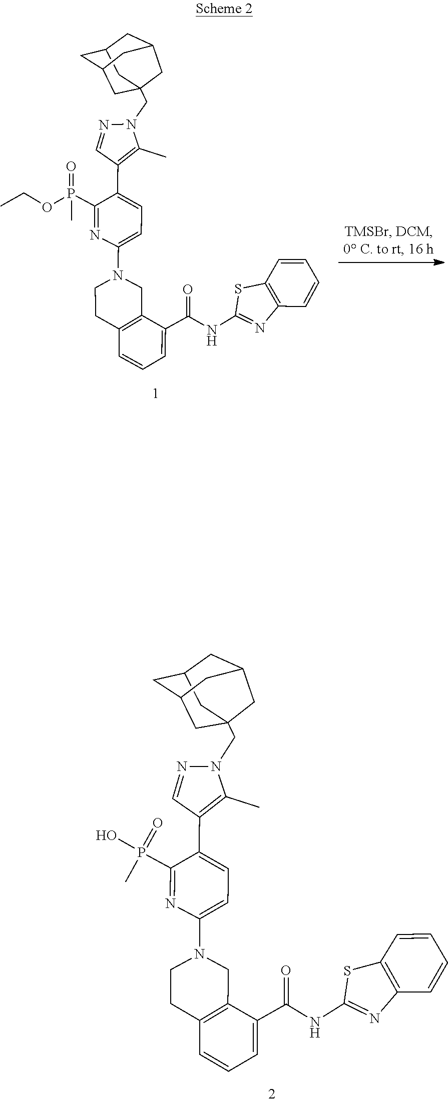 [SMILES: CCOP(C)(=O)c1nc(N2CCc3cccc(C(=O)Nc4nc5ccccc5s4)c3C2)ccc1-c1cnn(CC23CC4CC(CC(C4)C2)C3)c1C ; Cc1c(-c2ccc(N3CCc4cccc(C(=O)Nc5nc6ccccc6s5)c4C3)nc2P(C)(=O)O)cnn1CC12CC3CC(CC(C3)C1)C2]

Scheme 2 illustrates the preparation of (3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinic acid (2).

### Example 2: (3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinic acid (2)

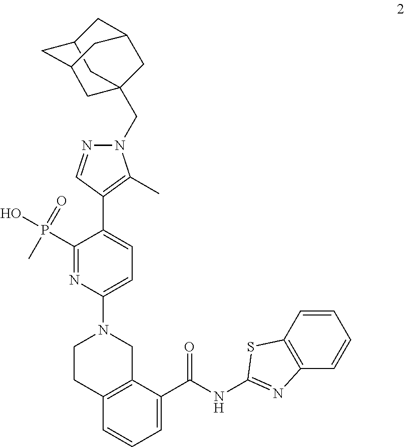 [SMILES: Cc1c(-c2ccc(N3CCc4cccc(C(=O)Nc5nc6ccccc6s5)c4C3)nc2P(C)(=O)O)cnn1CC12CC3CC(CC(C3)C1)C2]

To a stirred solution of ethyl(3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinate (860 mg, 1.19 mmol) in dichloromethane (30 mL) (1) was added dropwise TMSBr (1.82 g, 11.9 mmol) slowly at 0° C. The reaction mixture was stirred at room temperature for 16 h. The reaction mixture was quenched with MeOH (20 mL). The resulting mixture was stirred at room temperature for 1 h, and then concentrated under vacuum. The residue was purified by column chromatography on silica gel eluting with methanol in dichloromethane (0 to 5%) to afford (3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinic acid (720 mg, 87%) (2) as a yellow solid. 70 mg of (3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinic acid (2) was further purified by Prep-HPLC with the following conditions: Column: XBridge Prep OBD C18 Column, 30×150 mm, 5 um; Mobile Phase A: Water (10 mmol/L NH₄HCO₃), Mobile Phase B: Acetonitrile; Flow rate: 60 mL/min; Gradient: 32% B to 62% B in 13 min; Wave Length: 254 nm/220 nm; RT (min): 8.02 to afford (3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinic acid (45.5 mg, 65%) (2) as a white solid. ¹H NMR (400 MHZ, CD3OD) δ 7.92 (d, J=7.6 Hz, 1H), 7.77 (d, J=8.0 Hz, 1H), 7.68 (s, 1H), 7.65-7.60 (m, 1H), 7.50-7.32 (m, 5H), 7.02 (d, J=8.4 Hz, 1H), 5.12 (s, 2H), 3.94 (t, J=6.0 Hz, 2H), 3.77 (s, 2H), 3.11 (t, J=6.0 Hz, 2H), 2.19 (s, 3H), 1.99 (s, 3H), 1.80-1.72 (m, 3H), 1.71-1.61 (m, 9H), 1.38 (d, J=14.8 Hz, 3H). ESI-MS [M+H]⁺calcd for (C₃₈H₄₁N₆O₃PS): 693.27; found: 693.45.

Scheme 3 illustrates the preparation of compound 4-(((2S,3S,4R,5S,6S)-3,4,5-trihydroxy-6-methyltetrahydro-2H-pyran-2-yl)oxy) benzyl(3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinate (3) and key intermediate (2S,3S,4R,5R,6S)-2-(4-(chloromethyl) phenoxy)-6-methyltetrahydro-2H-pyran-3,4,5-triyl triacetate (114)

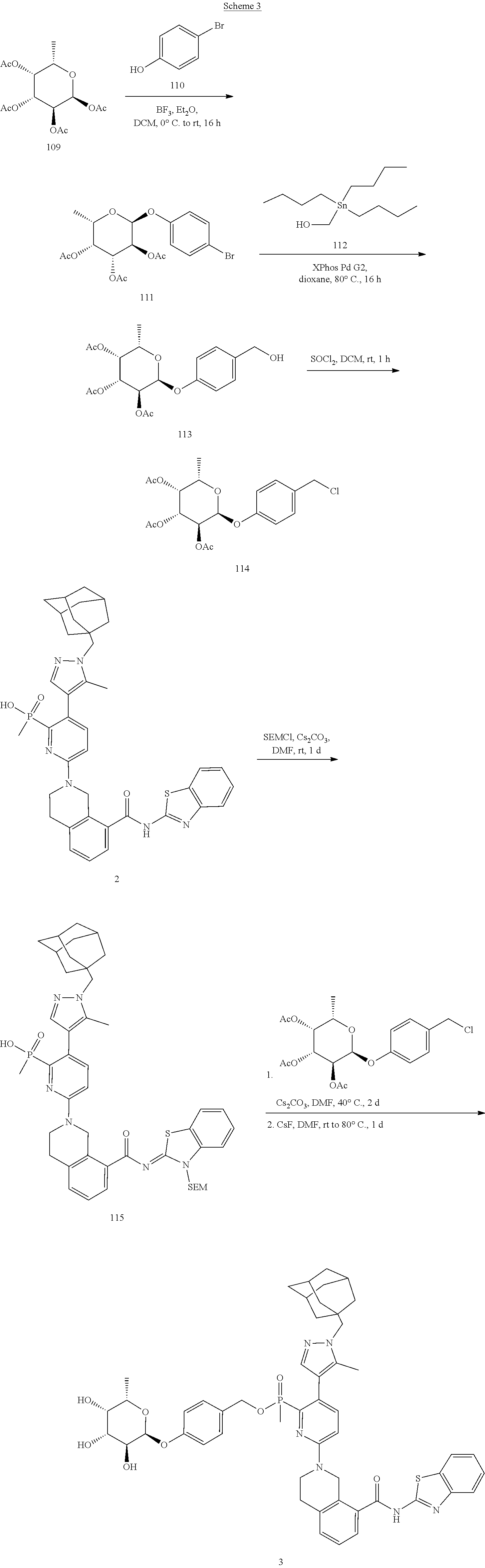

### (2S,3S,4R,5R,6S)-2-(4-bromophenoxy)-6-methyltetrahydro-2H-pyran-3,4,5-triyl triacetate (111)

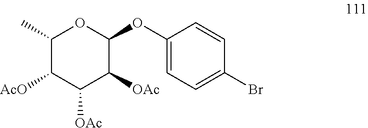 [SMILES: CC(=O)O[C@H]1[C@H](OC(C)=O)[C@H](Oc2ccc(Br)cc2)O[C@@H](C)[C@H]1OC(C)=O]

To a stirred mixture of (2S,3S,4R,5R,6S)-6-methyltetrahydro-2H-pyran-2,3,4,5-tetrayl tetraacetate (5.0 g, 15.04 mmol) (109) and 4-bromophenol (5.17 g, 30.08 mmol) (110) in dichloromethane (150 mL) under nitrogen was added BF₃·Et₂O (6.83 g, 48.15 mmol) at 0° C. The reaction mixture was stirred at room temperature for 16 h. The reaction mixture was quenched with water (150 mL) and extracted with ethyl acetate (3×150 mL). The combined organic layers was washed with brine (200 mL), dried over sodium sulfate and concentrated under vacuum. The residue was purified by column chromatography on silica gel eluting with ethyl acetate in petroleum ether (0 to 16%) to afford (2S,3S,4R,5R,6S)-2-(4-bromophenoxy)-6-methyltetrahydro-2H-pyran-3,4,5-triyl triacetate (4.4 g, 74%) (111) as a yellow oil. ¹H NMR (400 MHZ, CDCl₃) δ 7.39 (d, J=9.2 Hz, 2H), 6.94 (d, J=8.8 Hz, 2H), 5.69 (d, J=3.6 Hz, 1H), 5.55 (dd, J=11.2, 3.6 Hz, 1H), 5.35 (d, J=2.8 Hz, 1H), 5.26 (dd, J=10.8, 3.6 Hz, 1H), 4.23 (q, J=6.4 Hz, 1H), 2.19 (s, 3H), 2.06 (s, 3H), 2.03 (s, 3H), 1.12 (d, J=6.4 Hz, 3H). ESI-MS [M+NH₄]+calcd for (C18H₂₁BrO₈): 462.04, 464.04; found: 462.05, 464.05.

### (2S,3S,4R,5R,6S)-2-(4-(hydroxymethyl) phenoxy)-6-methyltetrahydro-2H-pyran-3,4,5-triyl triacetate (113)

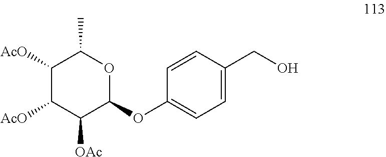 [SMILES: CC(=O)O[C@H]1[C@H](OC(C)=O)[C@H](Oc2ccc(CO)cc2)O[C@@H](C)[C@H]1OC(C)=O]

A mixture of (2S,3S,4R,5R,6S)-2-(4-bromophenoxy)-6-methyltetrahydro-2H-pyran-3,4,5-triyl triacetate (4.4 g, 9.88 mmol) (111), (tributylstannyl) methanol (4.76 g, 14.82 mmol) (112) and XPhos Pd G2 (1.17 g, 1.48 mmol) in dioxane (180 mL) was degassed and backfilled with nitrogen for five times. The reaction mixture was stirred at 80° C. for 16 h. The cooled reaction mixture was diluted with ethyl acetate (200 mL). The solids were filtered out. The filtrate was concentrated under vacuum. The residue was purified by column chromatography on silica gel eluting with ethyl acetate in petroleum ether (0 to 80%) to afford (2S,3S,4R,5R,6S)-2-(4-(hydroxymethyl) phenoxy)-6-methyltetrahydro-2H-pyran-3,4,5-triyl triacetate (3.5 g, 89%) (113) as a yellow oil. ¹H NMR (400 MHZ, CDCl₃) δ 7.30 (d, J=8.8 Hz, 2H), 7.05 (d, J=8.4 Hz, 2H), 5.73 (d, J=3.6 Hz, 1H), 5.58 (dd, J=10.8, 3.2 Hz, 1H), 5.36 (d, J=3.2 Hz, 1H), 5.27 (dd, J=10.8, 3.6 Hz, 1H), 4.64 (s, 2H), 4.35-4.23 (m, 1H), 2.19 (s, 3H), 2.06 (s, 3H), 2.03 (s, 3H), 1.12 (d, J=6.8 Hz, 3H). ESI-MS [M+NH₄]+calcd for (C₁₉H₂₄₀₉): 414.14; found: 414.20.

### (2S,3S,4R,5R,6S)-2-(4-(chloromethyl) phenoxy)-6-methyltetrahydro-2H-pyran-3,4,5-triyl triacetate (114)

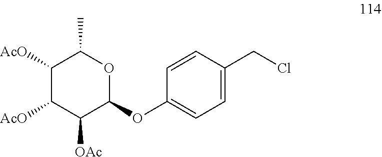 [SMILES: CC(=O)O[C@H]1[C@H](OC(C)=O)[C@H](Oc2ccc(CCl)cc2)O[C@@H](C)[C@H]1OC(C)=O]

To a stirred solution of (2S,3S,4R,5R,6S)-2-(4-(hydroxymethyl) phenoxy)-6-methyltetrahydro-2H-pyran-3,4,5-triyl triacetate (100 mg, 0.25 mmol) (113) in dichloromethane (5 mL) was added SOCl₂ (300 mg, 2.52 mmol). The reaction mixture was stirred at room temperature for 1 h. The reaction mixture was concentrated under vacuum to afford (2S,3S,4R,5R,6S)-2-(4-(chloromethyl) phenoxy)-6-methyltetrahydro-2H-pyran-3,4,5-triyl triacetate (130 mg, crude) (114) as a colorless oil, which was used directly in the next step without further purification.

### (3-(1-(((3r,5r,7r)-adamantan-1-yl)methyl)-5-methyl-1H-pyrazol-4-yl)-6-(8-(((Z)-3-((2-(trimethylsilyl) ethoxy)methyl) benzo[d]thiazol-2 (3H)-ylidene)carbamoyl)-3,4-dihydroisoquinolin-2 (1H)-yl) pyridin-2-yl)(methyl)phosphinic acid (115)

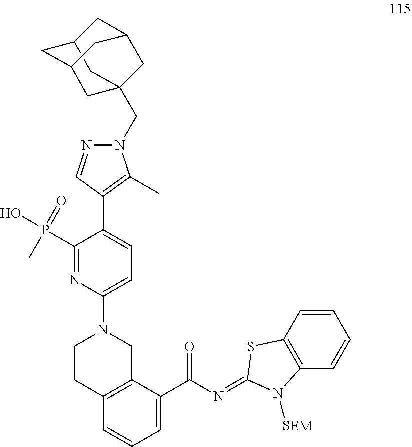 [SMILES: Cc1c(-c2ccc(N3CCc4cccc(C(=O)/N=c5\sc6ccccc6n5COCC[Si](C)(C)C)c4C3)nc2P(C)(=O)O)cnn1CC12CC3CC(CC(C3)C1)C2]

To a stirred mixture of (3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinic acid (500 mg, 0.722 mmol) (2) and Cs₂CO₃ (471 mg, 1.44 mmol) in DMF (20 mL) was added SEMCl (121 mg, 0.72 mmol) at 0° C. The reaction mixture was stirred at room temperature for one day. The reaction mixture was quenched with water (50 mL) and extracted with ethyl acetate (3×50 mL). The combined organic layers was washed with brine (100 mL), dried over Na₂SO₄ and concentrated under vacuum. The residue was purified by reversed phase column chromatography eluting with Acetonitrile in water (5% to 100%) to afford (3-(1-(((3r,5r,7r)-adamantan-1-yl)methyl)-5-methyl-1H-pyrazol-4-yl)-6-(8-(((Z)-3-((2-(trimethylsilyl) ethoxy)methyl) benzo[d]thiazol-2 (3H)-ylidene)carbamoyl)-3,4-dihydroisoquinolin-2 (1H)-yl) pyridin-2-yl)(methyl)phosphinic acid (170 mg, crude) (115) as a yellow solid. ESI-MS [M+H]⁺calcd for (C₄₄H₅₅N₆₀₄PSSi): 823.35; found: 823.30.

### Example 3:4-(((2S,3S,4R,5S,6S)-3,4,5-trihydroxy-6-methyltetrahydro-2H-pyran-2-yl)oxy) benzyl(3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinate (3)

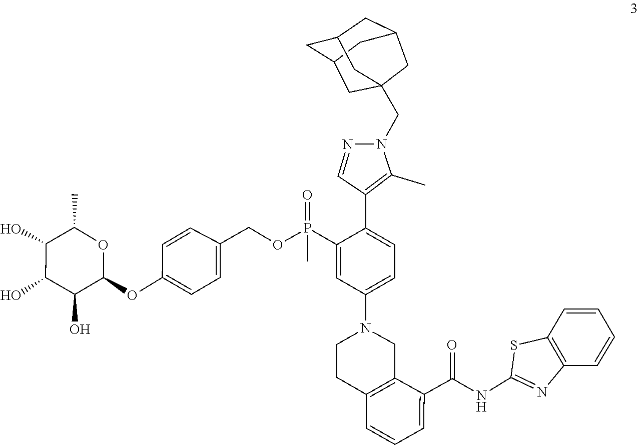 [SMILES: Cc1c(-c2ccc(N3CCc4cccc(C(=O)Nc5nc6ccccc6s5)c4C3)cc2P(C)(=O)OCc2ccc(O[C@@H]3O[C@@H](C)[C@@H](O)[C@@H](O)[C@@H]3O)cc2)cnn1CC12CC3CC(CC(C3)C1)C2]

To a stirred solution of (3-(1-(((3r,5r,7r)-adamantan-1-yl)methyl)-5-methyl-1H-pyrazol-4-yl)-6-(8-(((Z)-3-((2-(trimethylsilyl) ethoxy)methyl) benzo[d]thiazol-2 (3H)-ylidene)carbamoyl)-3,4-dihydroisoquinolin-2 (1H)-yl) pyridin-2-yl)(methyl)phosphinic acid (100 mg, 0.12 mmol) in DMF (6 mL) was added Cs₂CO₃ (198 mg, 0.61 mmol). After stirring at room temperature for 30 min, (2S,3S,4R,5R,6S)-2-(4-(chloromethyl) phenoxy)-6-methyltetrahydro-2H-pyran-3,4,5-triyl triacetate (151 mg, 0.36 mmol) was added. The reaction mixture was stirred at 40° C. for 2 days. The cooled reaction mixture was quenched with water (40 mL) and extracted with ethyl acetate (3×40 mL). The combined organic layers was washed with brine (50 mL), dried over Na₂SO₄ and concentrated under vacuum. The residue was purified by Prep-TLC (EtOAc) to afford (2S,3S,4R,5R,6S)-2-(4-((((3-(1-(((3r,5r,7r)-adamantan-1-yl)methyl)-5-methyl-1H-pyrazol-4-yl)-6-(8-(((Z)-3-((2-(trimethylsilyl) ethoxy)methyl) benzo[d]thiazol-2 (3H)-ylidene)carbamoyl)-3,4-dihydroisoquinolin-2 (1H)-yl) pyridin-2-yl)(methyl)phosphoryl)oxy) methyl) phenoxy)-6-methyltetrahydro-2H-pyran-3,4,5-triyl triacetate (60 mg, crude) (116) as a yellow solid. ESI-MS [M+H]⁺calcd for (C₆₃H₇₇N₆₀₁₂PSSi): 1201.48; found: 1201.50.

To a solution of (2S,3S,4R,5R,6S)-2-(4-((((3-(1-(((3r,5r,7r)-adamantan-1-yl)methyl)-5-methyl-1H-pyrazol-4-yl)-6-(8-(((Z)-3-((2-(trimethylsilyl) ethoxy)methyl) benzo[d]thiazol-2 (3H)-ylidene)carbamoyl)-3,4-dihydroisoquinolin-2 (1H)-yl) pyridin-2-yl)(methyl)phosphoryl)oxy) methyl) phenoxy)-6-methyltetrahydro-2H-pyran-3,4,5-triyl triacetate (60 mg, 0.05 mmol) (116) in DMF (3 mL) was added CsF (46 mg, 0.31 mmol). The reaction mixture was stirred at 80° C. for 1 day. The cooled reaction mixture was quenched with water (20 mL) and extracted with ethyl acetate (3×30 mL).

The combined organic layers was washed with brine (30 mL), dried over Na₂SO₄ and concentrated under vacuum. The residue was purified by Prep-HPLC with the following conditions: Column: XBridge Prep OBD C18 Column, 30×150 mm, 5 um; Mobile Phase A: Water (10 mmol/L NH₄HCO₃), Mobile Phase B: Acetonitrile; Flow rate: 60 mL/min; Gradient: 47% B to 77% B in 10 min; Wave Length: 254 nm/220 nm; RT (min): 7.17 to afford 4-(((2S,3S,4R,5S,6S)-3,4,5-trihydroxy-6-methyltetrahydro-2H-pyran-2-yl)oxy) benzyl(3-(1-(((3r,5r,7r)-adamantan-1-yl)methyl)-5-methyl-1H-pyrazol-4-yl)-6-(8-(benzo[d]thiazol-2-ylcarbamoyl)-3,4-dihydroisoquinolin-2 (1H)-yl) pyridin-2-yl)(methyl)phosphinate (2.9 mg, 6%) (3) as a white solid. ¹H NMR (400 MHZ, DMSO-d₆) δ 12.89 (brs, 1H), 8.16 (d, J=8.0 Hz, 1H), 7.93 (d, J=8.0 Hz, 1H), 7.78 (d, J=7.2 Hz, 1H), 7.69-7.57 (m, 4H), 7.56-7.46 (m, 2H), 7.25-7.15 (m, 3H), 7.12-7.04 (m, 2H), 5.57-5.48 (m, 1H), 5.29-5.13 (m, 2H), 5.07-4.96 (m, 1H), 4.92-4.80 (m, 2H), 4.78-4.71 (m, 1H), 4.65-4.56 (m, 1H), 4.07-3.97 (m, 3H), 3.96-3.83 (m, 4H), 3.73 (s, 1H), 3.27-3.16 (m, 2H), 2.26 (s, 3H), 2.04 (s, 3H), 1.83-1.72 (m, 3H), 1.72-1.54 (m, 12H), 1.21 (d, J=6.4 Hz, 3H). ESI-MS [M+H]⁺calcd for (C₅₁H₅₇N₆O₈PS): 945.37; found: 945.30.

Scheme 4 illustrates the preparation of key intermediate (Z)—N-(6-chloro-4-methylpyridazin-3-yl)-3-((2-(trimethylsilyl) ethoxy)methyl) benzo[d]thiazol-2 (3H)-imine (120)

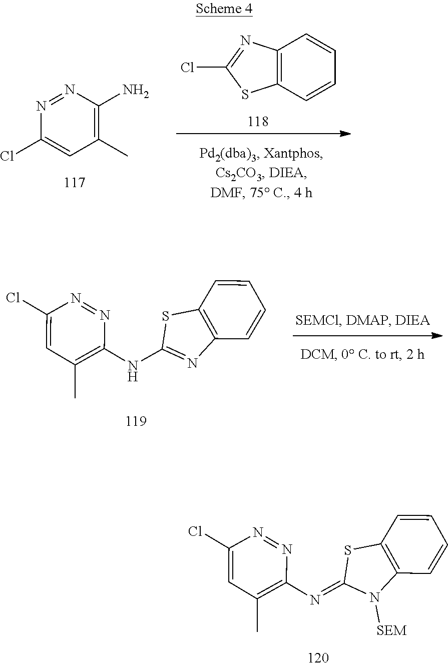

### N-(6-chloro-4-methylpyridazin-3-yl)benzo[d]thiazol-2-amine (119)

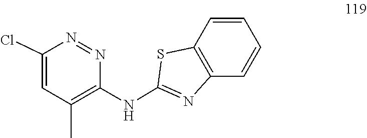 [SMILES: Cc1cc(Cl)nnc1Nc1nc2ccccc2s1]

A mixture of 6-chloro-4-methylpyridazin-3-amine (4.30 g, 29.95 mmol) (117), 2-chlorobenzo[d]thiazole (5.59 g, 32.95 mmol) (118), Cs₂CO₃ (29.20 g, 89.85 mmol), DIEA (11.59 g, 89.85 mmol), Pd₂(dba)₃ (1.37 g, 1.50 mmol) and XantPhos (1.73 g, 3.00 mmol) in DMF (120 mL) was degassed and backfilled with nitrogen for five times. The reaction mixture was heated at 75° C. for 4 h. The cooled reaction mixture was quenched with water (300 mL) and extracted with ethyl acetate (3×200 mL). The combined organic layers was washed with water (150 mL) and brine (150 mL), dried over sodium sulfate and concentrated under vacuum. The residue was purified by column chromatography on silica gel eluting with ethyl acetate in petroleum ether (0 to 33%) to afford N-(6-chloro-4-methylpyridazin-3-yl)benzo[d]thiazol-2-amine (6.5 g, 78%) (119) as a light yellow solid. 1HNMR (400 MHZ, DMSO-d₆) δ 7.92-7.80 (m, 1H), 7.65 (s, 1H), 759-7.47 (m, 1H), 7.39 (t, J=7.4 Hz, 1H), 7.22 (t, J=7.4 Hz, 1H), 2.37 (s, 3H). ESI-MS [M+H]⁺calcd for (C₁₂H₉ClN₄S): 277.02, 279.02; found: 277.00, 279.00.

### (Z)—N-(6-chloro-4-methylpyridazin-3-yl)-3-((2-(trimethylsilyl) ethoxy)methyl) benzo[d]thiazol-2 (3H)-imine (120)

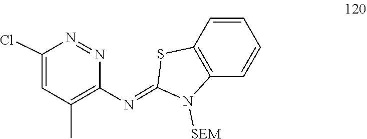 [SMILES: Cc1cc(Cl)nnc1/N=c1\sc2ccccc2n1COCC[Si](C)(C)C]

To a stirred mixture of N-(6-chloro-4-methylpyridazin-3-yl)benzo[d]thiazol-2-amine (4.30 g, 15.54 mmol) (119), DMAP (569 mg, 4.66 mmol) and DIEA (10.02 g, 77.69 mmol) in DCM (100 mL) under nitrogen was added dropwise SEM-C₁ (3.89 g, 23.31 mmol) at 0° C. After addition, the reaction mixture was stirred at room temperature for 2 h. The reaction mixture was quenched with water (120 mL) and extracted with dichloromethane (3×100 mL). The combined organic layers was washed with water (120 mL) and brine (120 mL), dried over anhydrous sodium sulfate and concentrated under vacuum. The residue was purified by column chromatography on silica gel eluting with ethyl acetate in petroleum ether (0 to 25%) to afford (Z)—N-(6-chloro-4-methylpyridazin-3-yl)-3-((2-(trimethylsilyl) ethoxy)methyl) benzo[d]thiazol-2 (3H)-imine (5.4 g, 85%) (120) as a yellow solid. ¹H NMR (400 MHZ, DMSO-d₆) δ 7.87-7.80 (m, 1H), 7.68 (s, 1H), 7.53-7.42 (m, 2H), 7.31-7.21 (m, 1H), 5.87 (s, 2H), 3.71 (t, J=7.8 Hz, 2H), 2.37 (s, 3H), 0.90 ((t, J=7.8 Hz, 2H), −0.13 (s, 9H). ESI-MS [M+H]⁺calcd for (C18H₂₃ClN₄OSSi) 407.11, 409.11; found: 407.15, 409.15.

### Scheme 5 illustrates the preparation of (2-((6-(benzo[d]thiazol-2-ylamino)-5-methylpyridazin-3-yl)(methyl) amino)-5-(3-(4-(3-(dimethylamino) prop-1-yn-1-yl)-2-fluorophenoxy) propyl)thiazol-4-yl)(methyl)phosphinic acid (4)

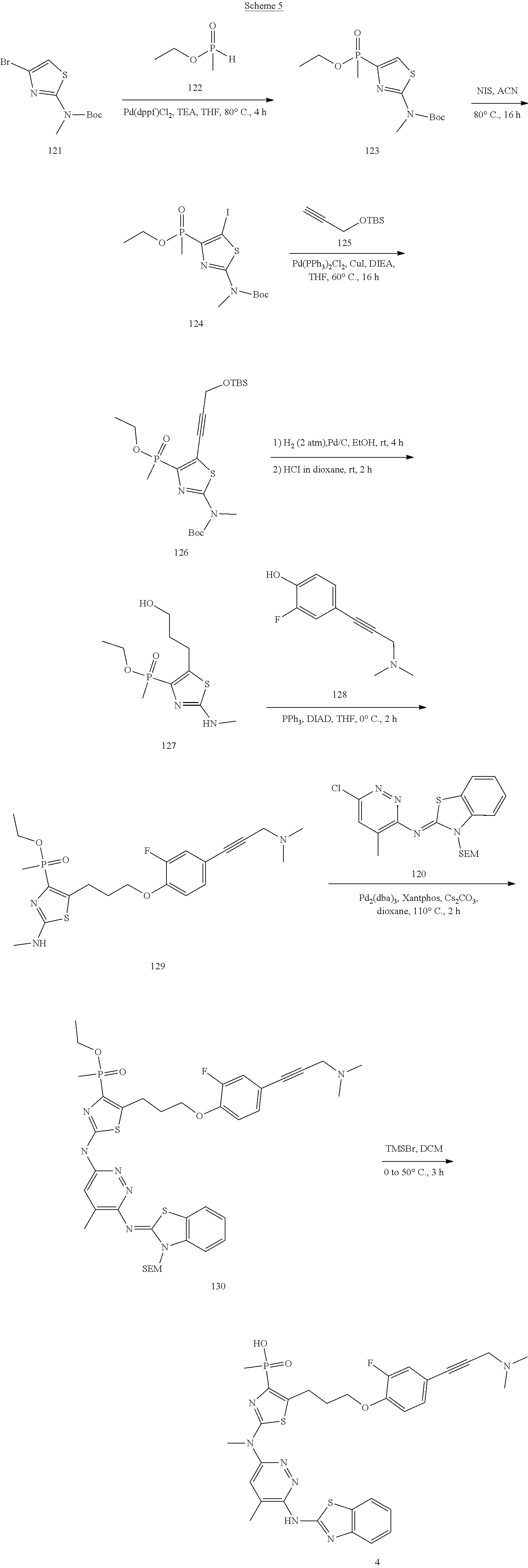

### tert-butyl(4-(ethoxy(methyl)phosphoryl)thiazol-2-yl)(methyl)carbamate (123)

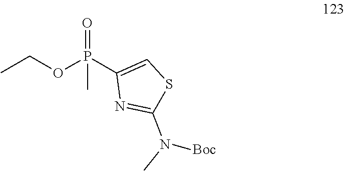 [SMILES: CCOP(C)(=O)c1csc(N(C)C(=O)OC(C)(C)C)n1]

To a mixture of tert-butyl(4-bromothiazol-2-yl)(methyl)carbamate (1.00 g, 3.41 mmol) (121), ethyl methylphosphinate (737 mg, 6.82 mmol) (122) and Pd(dppf)Cl₂ (500 mg, 0.68 mmol) in THF (20 mL) under nitrogen was added triethylamine (1.04 g, 10.23 mmol). The reaction mixture was stirred under nitrogen at 80° C. for 4 h. The cooled reaction mixture was quenched with water (50 mL) and extracted with ethyl acetate (3×50 mL). The combined organic layers was washed with water (50 mL) and brine (50 mL), dried over sodium sulfate and concentrated under vacuum. The residue was purified by column chromatography on silica gel eluting with ethyl acetate in dichloromethane (0 to 30%) to afford tert-butyl(4-(ethoxy(methyl)phosphoryl)thiazol-2-yl)(methyl)carbamate (1.0 g, 92%) (123) as an off-white solid. ¹H NMR (400 MHZ, DMSO-d₆) δ 7.89 (d, J=4.6 Hz, 1H), 4.00-3.75 (m, 2H), 3.47 (s, 3H), 1.63 (d, J=15.2 Hz, 3H), 1.53 (s, 9H), 1.18 (t, J=7.0 Hz, 3H). ESI-MS [M+H]⁺calcd for (C₁₂H₂₁N₂O₄PS): 321.10; found: 321.10.

### tert-butyl(4-(ethoxy(methyl)phosphoryl)-5-iodothiazol-2-yl)(methyl)carbamate (124)

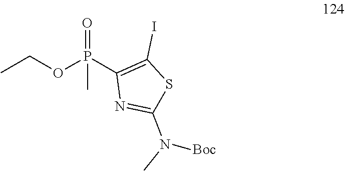 [SMILES: CCOP(C)(=O)c1nc(N(C)C(=O)OC(C)(C)C)sc1I]

To a stirred solution of tert-butyl(4-(ethoxy(methyl)phosphoryl)thiazol-2-yl)(methyl)carbamate (960 mg, 3.00 mmol) (123) in acetonitrile (25 mL) was added NIS (1.35 g, 5.99 mmol). The reaction mixture was stirred at 80° C. for 16 h. The cooled reaction mixture was quenched with water (50 mL) and extracted with ethyl acetate (3×50 mL). The combined organic layers was washed with brine (2×50 mL), dried over sodium sulfate and concentrated under vacuum. The residue was purified by column chromatography on silica gel eluting with ethyl acetate in dichloromethane (0 to 35%) to afford tert-butyl(4-(ethoxy(methyl)phosphoryl)-5-iodothiazol-2-yl)(methyl)carbamate (1.2 g, 89%) (124) as a light-yellow solid. ¹H NMR (400 MHz, DMSO-d₆) δ 4.06-3.81 (m, 2H), 3.43 (s, 3H), 1.70 (d, J=15.2 Hz, 3H), 1.52 (s, 9H), 1.22 (t, J=7.0 Hz, 3H). ESI-MS [M+H]⁺calcd for (C₁₂H₂O IN₂O₄PS): 446.99; found: 447.00.

### tert-butyl(5-(3-((tert-butyldimethylsilyl)oxy) prop-1-yn-1-yl)-4-(ethoxy(methyl)phosphoryl)thiazol-2-yl)(methyl)carbamate (126)

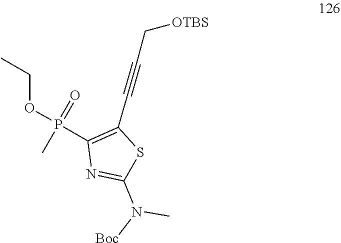 [SMILES: CCOP(C)(=O)c1nc(N(C)C(=O)OC(C)(C)C)sc1C#CCO[Si](C)(C)C(C)(C)C]

To a solution tert-butyl(4-(ethoxy(methyl)phosphoryl)-5-iodothiazol-2-yl)(methyl)carbamate (900 mg, 2.02 mmol) (124), tert-butyldimethyl(prop-2-yn-1-yloxy) silane (687 mg, 4.03 mmol) (125) and DIEA (782. mg, 6.05 mmol) in THF (24 mL) under nitrogen were added Pd(PPh₃)₂Cl₂ (141 mg, 0.20 mmol) and Cul (77 mg, 0.40 mmol). The reaction mixture was degassed and backfilled with nitrogen five times and stirred at 60° C. for 16 h. The cooled reaction mixture was quenched with water (50 mL) and extracted with ethyl acetate (3×40 mL). The combined organic layers was washed with brine (2×50 mL), dried over sodium sulfate and concentrated under vacuum. The residue was purified by column chromatography on silica gel eluting with ethyl acetate in petroleum ether (0 to 50%) to afford tert-butyl(5-(3-((tert-butyldimethylsilyl)oxy) prop-1-yn-1-yl)-4-(ethoxy(methyl)phosphoryl)thiazol-2-yl)(methyl)carbamate (620 mg, 63%) (126) as a yellow solid. ESI-MS [M+H]⁺calcd for (C₂₁H₃₇N₂O₅PSSi) 489.19 found: 489.20.

### Ethyl(5-(3-hydroxypropyl)-2-(methylamino) thiazol-4-yl)(methyl)phosphinate (127)

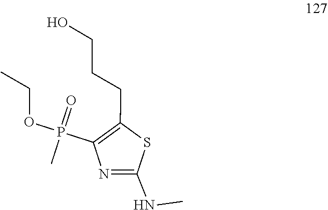 [SMILES: CCOP(C)(=O)c1nc(NC)sc1CCCO]

A mixture of tert-butyl(5-(3-((tert-butyldimethylsilyl)oxy) prop-1-yn-1-yl)-4-(ethoxy(methyl)phosphoryl)thiazol-2-yl)(methyl)carbamate (520 mg, 1.06 mmol) (126) and Pd/C(300 mg) in EtOH (16 mL) was stirred under hydrogen (2 atm) at room temperature for 4 h. The reaction mixture was filtered. The filtrate was concentrated under vacuum. To the residue was added 4 M HCl in dioxane (10 mL). The reaction mixture was stirred at room temperature for 2 h. The mixture was concentrated under a vacuum. The residue was diluted with water (10 mL) and basified with saturated aqueous sodium carbonate until pH=8. The aqueous layer was extracted with ethyl acetate (4×30 mL). The combined organic layers was washed with brine (40 mL), dried over anhydrous sodium sulfate and concentrated under vacuum. The residue was purified by column chromatography on silica gel eluting with ethyl acetate in petroleum ether (50% to 100%) to give ethyl(5-(3-hydroxypropyl)-2-(methylamino) thiazol-4-yl)(methyl)phosphinate (220 mg, 62%) (127) as an off-white solid. ESI-MS [M+H]⁺calcd for (C₁₀H₁₉N₂O₃PS). 279.09 found: 279.10.

### Ethyl(5-(3-(4-(3-(dimethylamino) prop-1-yn-1-yl)-2-fluorophenoxy) propyl)-2-(methylamino) thiazol-4-yl)(methyl)phosphinate (129)

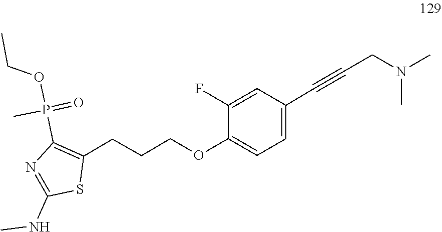

To a stirred mixture of ethyl(5-(3-hydroxypropyl)-2-(methylamino) thiazol-4-yl)(methyl)phosphinate (200 mg, 0.72 mmol) (127), 4-(3-(dimethylamino) prop-1-yn-1-yl)-2-fluorophenol (139 mg, 0.72 mmol) (128) and PPh₃ (283 mg, 1.08 mmol) in THF (15 mL) under nitrogen was added dropwise DIAD (218 mg, 1.08 mmol) at 0° C. The reaction mixture was stirred at 0° C. for 2 h. The reaction mixture was quenched with water (30 mL) and extracted with ethyl acetate (3×30 mL). The combined organic layers was washed with brine (2×40 mL), dried over sodium sulfate and concentrated under vacuum. The residue was purified by Prep-TLC(methanol/dichloromethane=1:10) to afford ethyl(5-(3-(4-(3-(dimethylamino) prop-1-yn-1-yl)-2-fluorophenoxy) propyl)-2-(methylamino) thiazol-4-yl)(methyl)phosphinate (250 mg, 77%) (129) as a light-yellow solid. ESI-MS [M+H]⁺calcd for (C₂₁H₂₉FN₃O₃PS). 454.17 found: 454.20.

### Ethyl(Z)-(5-(3-(4-(3-(dimethylamino) prop-1-yn-1-yl)-2-fluorophenoxy) propyl)-2-(methyl(5-methyl-6-((3-((2-(trimethylsilyl) ethoxy)methyl) benzo[d]thiazol-2 (3H)-ylidene) amino)pyridazin-3-yl) amino) thiazol-4-yl)(methyl)phosphinate (130)

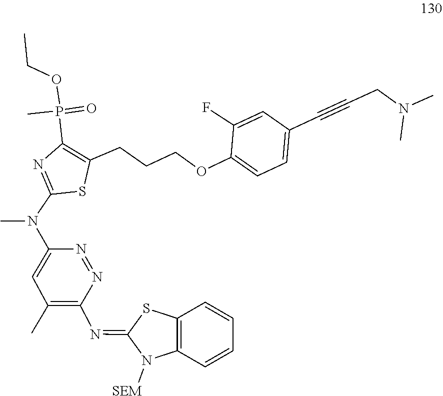

A mixture of ethyl(5-(3-(4-(3-(dimethylamino) prop-1-yn-1-yl)-2-fluorophenoxy) propyl)-2-(methylamino) thiazol-4-yl)(methyl)phosphinate (220 mg, 0.49 mmol) (129), (Z)—N-(6-chloro-4-methylpyridazin-3-yl)-3-((2-(trimethylsilyl) ethoxy)methyl) benzo[d]thiazol-2 (3H)-imine (237 mg, 0.58 mmol) (120), Pd₂(dba)₃ (46 mg, 0.05 mmol), XantPhos (58 mg, 0.10 mmol) and Cs₂CO₃ (475 mg, 1.46 mmol) in dioxane (15 mL) was degassed and backfilled with nitrogen for five times. The reaction mixture was heated at 110° C. for 2 h. The cooled reaction mixture was quenched with water (30 mL) and extracted with ethyl acetate (3×30 mL). The combined organic layers was washed with brine (2×40 mL), dried over sodium sulfate and concentrated under vacuum. The residue was purified by Prep-TLC(methanol/dichloromethane=1:10) to afford ethyl(Z)-(5-(3-(4-(3-(dimethylamino) prop-1-yn-1-yl)-2-fluorophenoxy) propyl)-2-(methyl(5-methyl-6-((3-((2-(trimethylsilyl) ethoxy)methyl) benzo[d]thiazol-2 (3H)-ylidene) amino)pyridazin-3-yl) amino) thiazol-4-yl)(methyl)phosphinat (300 mg, 75%) (130) as a light yellow solid. ESI-MS [M+H]⁺calcd for (C₃₉H₅₁FN₇₀₄PS2Si): 824.29; found: 824.60.

### Example 4: (2-((6-(benzo[d]thiazol-2-ylamino)-5-methylpyridazin-3-yl)(methyl) amino)-5-(3-(4-(3-(dimethylamino) prop-1-yn-1-yl)-2-fluorophenoxy) propyl)thiazol-4-yl)(methyl)phosphinic acid (4)

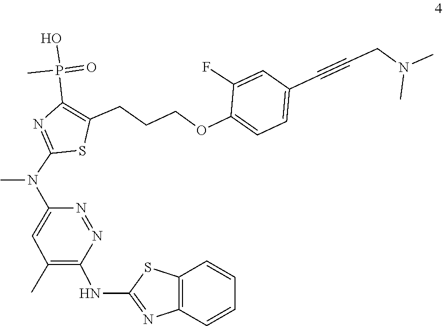

To a stirred solution of (Z)-(5-(3-(4-(3-(dimethylamino) prop-1-yn-1-yl)-2-fluorophenoxy) propyl)-2-(methyl(5-methyl-6-((3-((2-(trimethylsilyl) ethoxy)methyl) benzo[d]thiazol-2 (3H)-ylidene) amino)pyridazin-3-yl) amino) thiazol-4-yl)(methyl)phosphinat (280 mg, 0.34 mmol) in DCM (10 mL) was added bromotrimethylsilane (520 mg, 3.40 mmol) at 0° C. The reaction mixture was stirred at 50° C. for 3 h. The reaction mixture was quenched with methanol (30 mL) and stirred at 25° C. for 1 h. The resulting mixture was concentrated under vacuum. The residue was purified by Prep-HPLC with the following conditions: Column: XBridge Prep OBD C18 Column, 50×250 mm, 10 um; Mobile Phase A: Water (10 nmol/L NH₄HCO₃), Mobile Phase B: Acetonitrile; Flow rate: 60 mL/min; Gradient: 31% B to 61% B in 20 min; Wave Length: 254 nm/220 nm; RT (min): 15.57 to afford (2-((6-(benzo[d]thiazol-2-ylamino)-5-methylpyridazin-3-yl)(methyl) amino)-5-(3-(4-(3-(dimethylamino) prop-1-yn-1-yl)-2-fluorophenoxy) propyl)thiazol-4-yl)(methyl)phosphinic acid (38.6 mg, 17%) (4) as a yellow solid. ¹H NMR (400 MHZ, DMSO-d₆) δ 7.89 (d, J=7.7 Hz, 1H), 7.64 (s, 1H), 7.52 (d, J=8.0 Hz, 1H), 7.38 (t, J=7.7 Hz, 1H), 7.28 (d, J=11.7 Hz, 1H), 7.24-7.09 (m, 3H), 4.11 (t, J=6.0 Hz, 2H), 3.74 (s, 3H), 3.42 (s, 2H), 3.30 (t, J=7.1 Hz, 2H), 2.43 (s, 3H), 2.22 (s, 6H), 2.16-2.07 (m, 2H), 1.52 (d, J=14.3 Hz, 3H). ESI-MS [M+H]⁺calcd for (C₃₁H₃₃FN₇O₃PS2): 666.18; found: 666.20.

### Example 5: Senescence Assay Procedure and Results

Cells were maintained in DMEM media (high glucose, 4 mM L-glutamine, no sodium pyruvate) supplemented with 10% fetal bovine serum in a humidified incubator at 37° C. with 5% carbon dioxide. Senescence was induced via treatment with a sublethal concentration of chemotherapy (bleomycin, gemcitabine, doxorubicin, 5-fluorouridine, mitomycin C), followed by a rest period without chemotherapy similarly to what is reported by others in the field (e.g., Aoshiba et al., 2003 Eur Respir J 22:436-443). Senescence was qualified by low EdU (5-ethynyl-2′-deoxyuridine) incorporation and high SA-β-Gal (senescence-associated beta-galactosidase) staining compared to proliferating untreated cells. Senescent cells were plated into 96-well plates at a density of 5-15,000 cells/well. The next day, compounds were added in duplicate across ten different concentrations spanning roughly four Log10 units. Cells were incubated with the compounds for 24-72 hours, at which point viability was quantified using CellTiter-Glo (Promega) or XTT (R&D Systems). The viability for treated cells was determined relative to untreated control cells. The log of the compound concentration vs. normalized mean viability was fit to a four parameters Hill function with variable slope. The bottom was constrained to be greater than zero. The X mean value for Y=50 was interpolated to determine the absolute IC₅₀. The results in Table 1 indicate that many of the compounds are active in cells and can induce a dose dependent decrease in the viability of senescent A549 and IMR90 cells.

The results are presented in Table 1 below.

TABLE 1

| Com- | Absolute IC50 average | | |
|---|---|---|---|
| pound # | A549_SEN IC50 | IMR90_SEN IC50 | HACAT_SEN IC50 |
| 1 | *** | *** | **** |
| 2 | *** | ** | n.t. |
| 3 | *** | ** | n.t. |
| 4 | ** | * | n.t. |

* 1E−05 to 1E−06M,
** 1E−07 to 1E−06M,
*** 1E−08 to 1E−07M,
**** less than 1E−08M,
n.t. not evaluated.

What is claimed is:

1. A compound of structure: 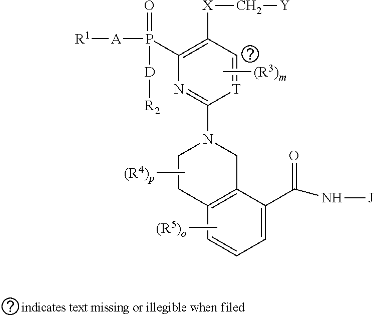
or salts, hydrates and solvates thereof wherein:
X is heteroaryl or substituted heteroaryl;
Y is alkyl, substituted alkyl, cycloalkyl, substituted cycloalkyl, heteroalkyl or substituted heteroalkyl;
A is —O—or —NR⁶—;
D is —O—or absent;
J is heteroaryl or substituted heteroaryl;
R¹, R² and R⁶ are independently-H, alkyl, alkenyl, aryl, substituted aryl, arylalkyl, substituted arylalkyl, cycloalkyl, substituted cycloalkyl, cycloalkenyl or substituted cycloalkenyl wherein substituted arylalkyl includes substitution with a carbohydrate derivative;
Each of R³ and R⁵ are independently halo, alkyl, alkenyl, alkynyl or haloalkyl;
R⁴ is halo, alkyl, alkenyl, alkynyl or haloalkyl or alternatively two R⁴ attached to the carbon atom form a cycloalkyl or cycloalkenyl ring;
m is 0, 1, 2 or 3;
n is 0 or 1;
o is 0, 1, 2 or 3; and
p is 0, 1, 2 or 3.

2. The compound of claim 1, wherein n is 1 and T is —C—.

3. The compound of claim 1, wherein n is 0 and T is —S—.

4. The compound of claim 1, wherein X is substituted heteroaryl.

5. The compound of claim 1, Y is cycloalkyl or substituted alkyl.

6. The compound of claim 1, wherein A is —O—.

7. The compound of claim 1, wherein R¹ is —H, alkyl or substituted arylalkyl.

8. The compound of claim 1, wherein D is absent.

9. The compound of claim 1, wherein J is heteroaryl.

10. The compound of claim 1, wherein m, o and p are 0.

11. The compound of claim 2, wherein X is substituted heteroaryl, Y is cycloalkyl, A is —O—, R¹ is —H, alkyl or substituted arylalkyl, D is alkyl, J is heteroaryl and m, o and p are 0.

12. The compound of claim 11 having the structure:

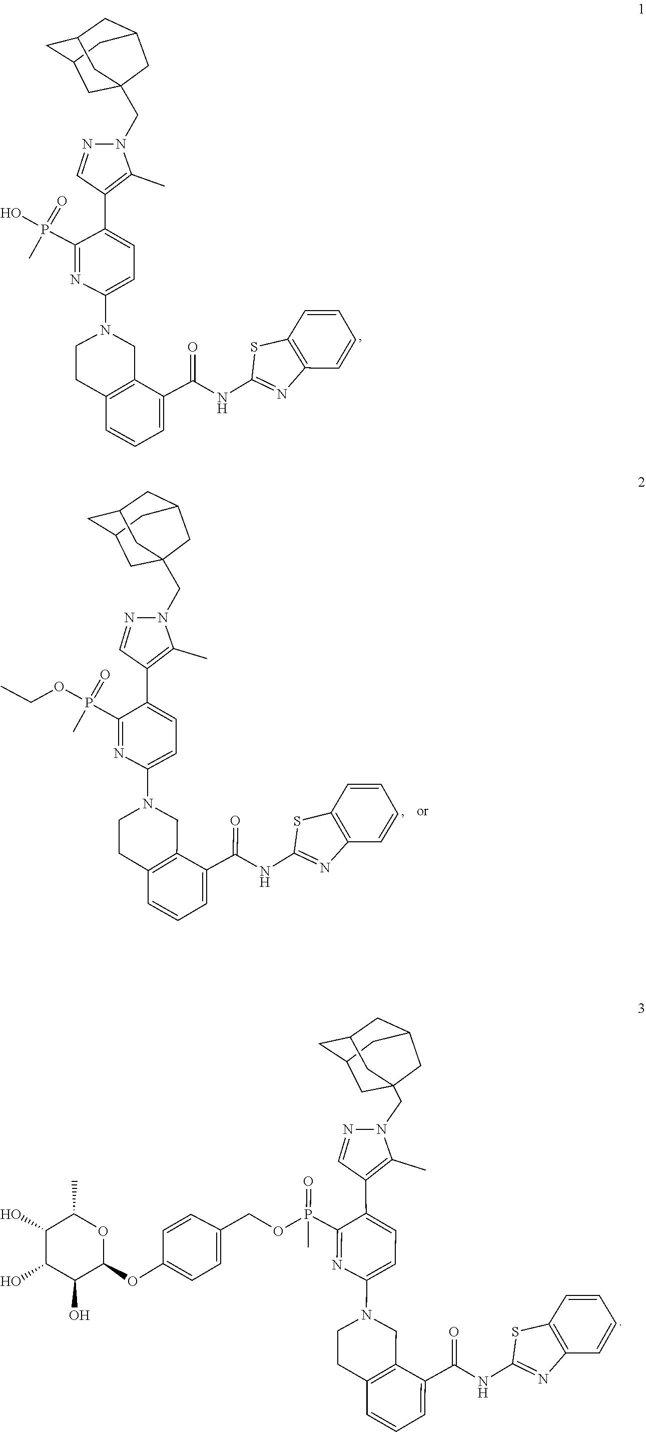 [SMILES: Cc1c(-c2ccc(N3CCc4cccc(C(=O)Nc5nc6ccccc6s5)c4C3)nc2P(C)(=O)O)cnn1CC12CC3CC(CC(C3)C1)C2 ; CCOP(C)(=O)c1nc(N2CCc3cccc(C(=O)Nc4nc5ccccc5s4)c3C2)ccc1-c1cnn(CC23CC4CC(CC(C4)C2)C3)c1C ; Cc1c(-c2ccc(N3CCc4cccc(C(=O)Nc5nc6ccccc6s5)c4C3)nc2P(C)(=O)OCc2ccc(O[C@@H]3O[C@@H](C)[C@@H](O)[C@@H](O)[C@@H]3O)cc2)cnn1CC12CC3CC(CC(C3)C1)C2]

13. The compound of claim 3, wherein X is substituted heteroaryl, Y is substituted alkyl, A is —O—, R¹ is —H or alkyl, D is alkyl, J is heteroaryl and m, o and p are 0.

14. The compound of claim 13 having the structure:

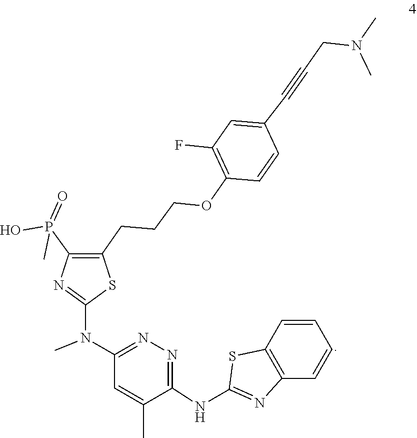 [SMILES: Cc1cc(N(C)c2nc(P(C)(=O)O)c(CCCOc3ccc(C#CCN(C)C)cc3F)s2)nnc1Nc1nc2ccccc2s1]

15. The compound of claim 1 and a pharmaceutically acceptable vehicle.

16. A method for treating a senescence-associated disease or disorder comprising administering to a subject in need thereof a therapeutically-effective amount of a compound of claim 1.

17. A compound of the structure: 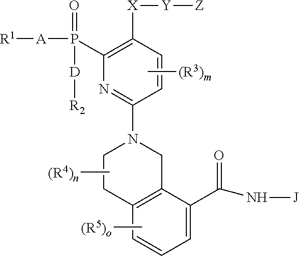
or salts, hydrates and solvates thereof where X is heteroaryl or substituted heteroaryl; Y is alkyldiyl, substituted alkyldiyl, heteroalkyldiyl or substituted heteroalkyldiyl; Z is cycloalkyl, substituted cycloalkyl, cycloalkenyl, substituted cycloalkenyl, aryl, substituted aryl, cycloheteroalkyl or substituted cycloheteroalkyl; A is —O—or —NR⁶—; D is —O—or absent; J is heteroaryl or substituted heteroaryl; R¹, R² and R⁶ are independently-H alkyl, alkenyl, aryl, substituted aryl, arylalkyl, substituted arylalkyl cycloalkyl, substituted cycloalkyl, cycloalkenyl or substituted cycloalkenyl wherein substituted arylalkyl includes substitution with a carbohydrate derivative; each R³ and R⁵ are independently halo, alkyl, alkenyl, alkynyl or haloalkyl; R⁴ is halo, alkyl, alkenyl, alkynyl or haloalkyl or alternatively two R⁴ attached to the carbon atom form a cycloalkyl or cycloalkenyl ring; m is 0, 1, 2 or 3, n is 0, 1, 2, 3, 4, 5 or 6; and
o is 0, 1, 2 or 3.

18. A compound of the structure: 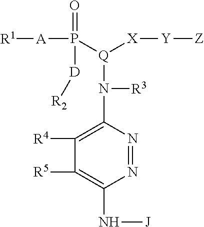
or salts, hydrates and solvates thereof where Q is heteroaryl or substituted heteroaryl; X is alkyldiyl, substituted alkyldiyl, heteroalkyldiyl or substituted heteroalkyldiyl; Y is —O—, —NR⁶—, or—CH₂—; Z is cycloalkyl, substituted cycloalkyl, cycloalkenyl, substituted cycloalkenyl, aryl, substituted aryl, cycloheteroalkyl, substituted cycloheteroalkyl heteroaryl or substituted heteroaryl; A is —O—or —NR⁷—; D is —O—or absent; J is heteroaryl or substituted heteroaryl; R¹, R², R³ and R⁷ are independently-H, alkyl, alkenyl, aryl, substituted aryl, arylalkyl or substituted arylalkyl where substituted arylalkyl includes substitution with a carbohydrate derivative; R⁴ and R⁵ are independently-H, alkyl, substituted alkyl, alkenyl, substituted alkenyl, cycloalkyl, substituted cycloalkyl or together with the atoms to which they are bonded form a cycloalkenyl or substituted cycloalkenyl group; and R⁶ is —H or alkyl.
